SEQUENCE LISTING (29 sequences)

SEQ ID NO: 1 - protein, 7 aa, synthetic construct
ASSLNIA

SEQ ID NO: 2 - protein, 7 aa, synthetic construct
WDANGKT

SEQ ID NO: 3 - protein, 7 aa, synthetic construct
GETRAPL

SEQ ID NO: 4 - protein, 9 aa, synthetic construct
CGHHPVYAC

SEQ ID NO: 5 - protein, 7 aa, synthetic construct
HAIYPRH

SEQ ID NO: 6 - protein, 9 aa, synthetic construct
  REGION 1..9: note = circular peptide
CQLFPLFRC

SEQ ID NO: 7 - protein, 22 aa, synthetic construct
  SITE 2: note = Acp
  SITE 5: note = bAla
  SITE 8: note = Acp
  SITE 16: note = Acp
  SITE 18: note = bAla
  SITE 20: note = Acp
  SITE 22: note = bAla
RXRRXRRXRFQILYRXRXRXRX

SEQ ID NO: 8 - protein, 8 aa, synthetic construct
  REGION 1..8: note = peptide fragment
FHCHFSPR

SEQ ID NO: 9 - protein, 15 aa, synthetic construct
  REGION 1..15: note = peptide fragment
SSFYCLEYFPSQMLR

SEQ ID NO: 10 - protein, 11 aa, synthetic construct
  REGION 1..11: note = peptide fragment
DVYLQGPIWAK

SEQ ID NO: 11 - protein, 9 aa, synthetic construct
  REGION 1..9: note = peptide fragment
HPPPQILIK

SEQ ID NO: 12 - protein, 7 aa, synthetic construct
  REGION 1..7: note = peptide fragment
NWHCDST

SEQ ID NO: 13 - protein, 7 aa, synthetic construct
  REGION 1..7: note = peptide fragment
NWHCDSQ

SEQ ID NO: 14 - protein, 7 aa, synthetic construct
  REGION 1..7: note = peptide fragment
DWHCDST

SEQ ID NO: 15 - protein, 7 aa, synthetic construct
  REGION 1..7: note = peptide fragment
RFHCHFS

SEQ ID NO: 16 - protein, 7 aa, synthetic construct
  REGION 1..7: note = peptide fragment
RFHSHWS

SEQ ID NO: 17 - protein, 7 aa, synthetic construct
  REGION 1..7: note = peptide fragment
HQGCLPP

SEQ ID NO: 18 - protein, 7 aa, synthetic construct
  REGION 1..7: note = peptide fragment
QEGSLPP

SEQ ID NO: 19 - protein, 7 aa, synthetic construct
  REGION 1..7: note = peptide fragment
TEGCLPA

SEQ ID NO: 20 - protein, 7 aa, synthetic construct
  REGION 1..7: note = peptide fragment
HEGCLPP

SEQ ID NO: 21 - protein, 7 aa, synthetic construct
  REGION 1..7: note = peptide fragment
SFYCLEY

SEQ ID NO: 22 - protein, 7 aa, synthetic construct
  REGION 1..7: note = peptide fragment
AFYCLEY

SEQ ID NO: 23 - protein, 7 aa, synthetic construct
  REGION 1..7: note = peptide fragment
SFFCLEY

SEQ ID NO: 24 - protein, 7 aa, synthetic construct
  REGION 1..7: note = peptide fragment
PGPCYRQ

SEQ ID NO: 25 - protein, 7 aa, synthetic construct
  REGION 1..7: note = peptide fragment
PGPSIKQ

SEQ ID NO: 26 - protein, 7 aa, synthetic construct
  REGION 1..7: note = peptide fragment
PGPMGRT

SEQ ID NO: 27 - protein, 7 aa, synthetic construct
  REGION 1..7: note = peptide fragment
PGPCFRQ

SEQ ID NO: 28 - protein, 7 aa, synthetic construct
  REGION 1..7: note = peptide fragment
PGPSYRQ

SEQ ID NO: 29 - protein, 7 aa, synthetic construct
  REGION 1..7: note = peptide fragment
PGPCVKQ

(19) United States
(12) Patent Application Publication
(10) Pub. No.: US 2025/0332286 A1
(43) Pub. Date: Oct. 30, 2025

(54) CHEMICALLY-MODIFIED ADENO-ASSOCIATED VIRUSES

(71) Applicants: CENTRE NATIONAL DE LA RECHERCHE SCIENTIFIQUE, PARIS (FR); NANTES UNIVERSITE, NANTES (FR); INSTITUT NATIONAL DE LA SANTÉ ET DE LA RECHERCHE MÉDICALE, PARIS (FR)

(72) Inventors: MATHIEU MEVEL, NANTES (FR); DAVID DENIAUD, SAINT HERBLAIN (FR); SEBASTIEN GOUIN, THOUARÉ SUR LOIRE (FR); DIMITRI ALVAREZ-DORTA, NANTES (FR); PIERRE-ALBAN LALYS, NANTES (FR)

(21) Appl. No.: 18/855,379

(22) PCT Filed: Apr. 7, 2023
(86) PCT No.: PCT/EP2023/059337

§ 371 Date: Oct. 9, 2024

(30) Foreign Application Priority Data
Apr. 11, 2022 (EP) 22305516.1

(51) Int. Cl. A61K 48/00; C12N 15/86

(52) U.S. Cl. CPC A61K 48/0058; A61K 48/0091; C12N 15/86; C12N 2750/14122; C12N 2750/14143; C12N 2750/14145

(57) ABSTRACT

The invention relates to chemically modified adeno-associated (AAV) viruses and their use in gene therapy.

### FIELD OF THE INVENTION

The invention relates to chemically modified adeno-associated (AAV) viruses and their use in gene therapy.

### BACKGROUND OF THE INVENTION

Gene therapy was originally developed to correct defective genes that underlie genetic diseases. Nowadays, gene therapy is more and more used in the treatment of a broad range of acquired diseases such as cancers.

Gene therapy is based on the therapeutic delivery of nucleic acid into a patient's cell nucleus. The nucleic acids may then be inserted into the genome of the targeted cell or may remain episomal. Delivery of a therapeutic nucleic acid to a subject's target cells can be carried-out by various methods, including the use of synthetic and viral vectors. Among the many viral vectors available (e.g, retrovirus, lentivirus, adenovirus, and the like), recombinant adeno-associated virus (AAV) is gaining popularity as a versatile vector for gene therapy, particularly for in vivo applications. The main advantages of recombinant AAV (rAAV) reside in their broad tropism, their high transduction efficacy, their persistent episomal expression and their high safety profile, in particular because wild-type AAV is not associated with any human diseases.

Human clinical trials with rAAV have demonstrated durable expression at therapeutic levels when targeting tissues such as retina, liver or motor neurons. Several clinical trials using rAAV as gene vector are ongoing for a wide type of disorders. The FDA and the EMA have recently authorized Voretigene neparvovec (Luxturna®), which is an adeno-associated viral vector serotype 2 (AAV2) capsid comprising a cDNA encoding for the human retinal pigment epithelium 65 kDa protein (hRPE65), for the treatment of vision loss due to inherited retinal dystrophy caused by confirmed biallelic RPE65 mutations. As a further example, Zolgensma® (onasemnogene abeparvovec-xioi) has just been approved by the FDA for the treatment of pediatric patients less than 2 years of age with spinal muscular atrophy (SMA). Zolgensma® is an AAV9 vector able to deliver a functional, non-mutated copy of the defective gene in SMA, namely the SMN1 gene, in motoneurons.

In spite of these success, certain clinical trials have shown some limitations of these vectors, in the treatment of certain diseases. Their first limitation lies on their immunogenicity. Because of their non-integrative nature, systemic gene therapy with AAV vectors, especially in paediatric patients, might be limited by tissue proliferation inducing a dilution of the vector over time. However, the re-administration of the vectors might be precluded by persistent neutralizing antibodies (Nabs) triggered following the first administration of the viral vector. Moreover, it was further shown that preexisting humoral immunity to certain AAV serotypes, especially AAV of serotype 2, are highly prevalent in humans. Anti-AAV neutralizing antibodies (NAbs) can completely prevent transduction in a target tissue, resulting in lack of efficacy, particularly when the vector is administered directly into the bloodstream. As a result, subjects seropositive to AAV-Nabs are generally excluded from gene therapy trials.

A further limitation of AAV lies on their broad tropism, which may result in transgene expression in other tissues other than those where transgene expression is desired.

AAV as gene vector may also suffer from a reduced therapeutic index. Sometimes, the administration of high dose of AAV is needed to achieve effective transduction. For instance, although AAV2 vectors can efficiently target the liver, the transgene expression can be restricted to a very small of the transfected hepatocytes due to intracellular proteasome-mediated degradation of the vectors, whereby high dose or AAV-2 may be required to achieve the sought therapeutic effect. Such high doses pose a challenge not only for vector production but also increases the risk of immune response, among which the induction of Nabs.

Several strategies have been proposed to overcome the drawbacks of AAV, especially those of the AAV of serotype 2 (AAV2) in gene therapy. Certain of them are based on the modification of the capsid proteins of the vectors.

The first option is to genetically modify the viral capsid. For instance, it was shown that mutations in surface-exposed tyrosine residues on AAV2 enable to circumvent phosphorylation and subsequent ubiquitination thereby avoiding proteasome-mediated degradation. (Zhong et al., PNAS, 2008, 105, 7827-7832; Markusic et al. Molecular Therapy, 2010, 18, 2048-2056). Chemical modifications of the viral capsids were also suggested in order to introduce a ligand on the capsid or mask certain exposed amino acids so as to modify the antigenicity, the tropism or the transduction efficacity of AAV. As a first strategy, it was proposed to genetically incorporate unnatural amino acids with modified side chains (e.g. as in WO2015/062516), a non-natural amino acid, such as an amino acid comprising an azido, is inserted into the capsid by genetic modification prior to a coupling step with a ligand by click reaction so as to change its tropism for the target cell. Another strategy resides in the direct chemical modification of the viral capsid without any preliminary site-directed mutagenesis of the capsid proteins.

In that matter, International patent application WO2017/212019 proposes a method for chemically modifying the AAV capsid by covalently coupling a ligand bearing an isothiocyanate group which reacts with an amino group present in an amino acid residue such as lysine or arginine.

WO2021/005210 described a method for chemically modified tyrosine residues present in the capsids by reaction with a ligand bearing an aryl diazonium or a PTAD moiety.

However, there is still a need for new methods enabling to modulate the properties of AAV when used as gene delivery vectors in gene therapy.

### SUMMARY OF THE INVENTION

The present invention relates to an adeno-associated Virus (AAV) having at least one chemically-modified cysteine residue in its capsid, wherein said chemically-modified cysteine residue is of formula (I):

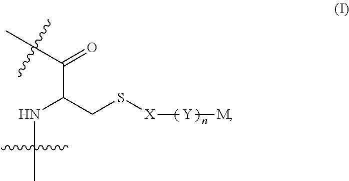

wherein:
   X is selected from the group consisting of:

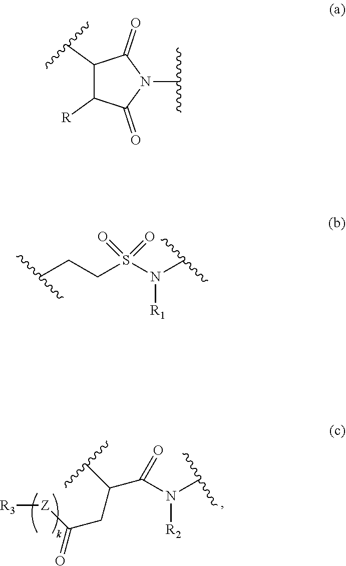

wherein
   Z is —O—, —S—, or —N(R₄)—,
   R₁, R₂, R₃ and R₄ are each independently chosen from a hydrogen atom, an alkyl group, an aryl group, a heteroaryl, said group being optionally substituted,
   k is 0 or 1,
   R is a hydrogen, a halogen, an alkyl group, an aryl group, a heteroaryl group, an alkoxy group, said group being optionally substituted,
   Y is a spacer,
   n is O or 1, and
   M is a functional moiety.

In some embodiments, X is of formula (b) or formula (c).

In preferred embodiments, the AAV is such that the chemically-modified cysteine residue is of formula (Ic):

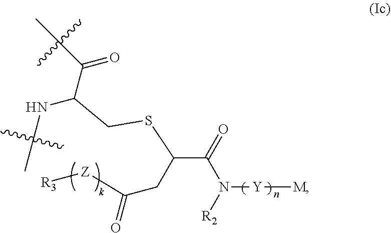

wherein Y, n, M, Z, k, R₂ and R₃ are as defined herein.

In some embodiments, the chemically-modified cysteine residue is of formula (Ic) wherein:
R₂ is hydrogen atom, a C₁-C₆ alkyl group, an aryl group comprising from 6 to 14 ring atoms, or a heteroaryl group comprising from 5 to 14 ring atoms, and/or
R₃ is selected from the group consisting of an aryl group comprising from 6 to 14 ring atoms and heteroaryl group comprising from 5 to 14 ring atoms, said aryl or heteroaryl group being optionally substituted by 1 to 3 substituents preferably chosen from halogens, —OH, NH₂, NO₂, C₁-C₃ alkyl, C₁-C₃ alkoxy, C₁-C₃ hydroxyalkyl, and C₁-C₃ haloalkyl, and/or
k is 0.

In other embodiments, the chemically-modified cysteine residue is of formula (Ic) wherein:
R₂ is hydrogen atom or a C₁-C₆ alkyl group, preferably H or a C₁-C₃ alkyl group
R₃ is an unsubstituted phenyl or a phenyl substituted by 1 to 3 substituents selected from halogens, —OH, NH₂, NO₂, C₁-C₃ alkyl, C₁-C₃ alkoxy, C₁-C₃ hydroxyalkyl, and C₁-C₃ haloalkyl, and
k is 0.

In some embodiments, Y in formula (I) is a spacer of formula (II):

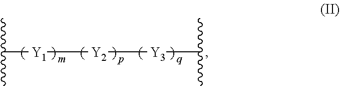

wherein:
m, p and q are each independently 0 or 1,
Y₁ is selected from the group consisting of an alkylene group, an arylene group, a heteroarylene group, said group being optionally substituted, preferably a phenylene group,
Y₂ is —C(═O)—NH, —C(═O)—O, —C(═O)—O—C(═O)—, O—(C═O)—, NH—C(═O)—, NH—C(═O)—NH, —O—C═O—O—, O, NH, —NH(C═S)—, or —(C═S)—NH—, preferably —(C═O)—NH—,
Y₃ is selected from the group consisting of polymers including homopolymers, copolymers and block polymers, peptides, oligosaccharides, saturated or unsaturated, branched or linear hydrocarbon chains, optionally interrupted by one or several heteroatoms and/or by a group chosen from —C(═O)—NH, —C(═O)—O, —C(═O)—O—C(═O)—, O—(C═O)—, NH—C(═O)—, NH—C(═O)—NH, —O—C(═O)—O—, —NH(C═S)—, and —(C═S)—NH-, and/or by one or more C₃-C₆ hydrocarbon cycle or C₂-C₆ heterocycle.

In some embodiments, M is a functional moiety comprising a group selected from a click-chemistry group, a steric shielding agent, a labelling agent, a targeting agent such as a cell-type specific ligand, a drug moiety, an oligonucleotide and combinations thereof.

In some embodiments, said chemically-modified cysteine residue of formula (I) is such that:
X is of formula (c),
n is 1,
M is a functional moiety, and
Y is a spacer of formula (II) wherein m is 0, p is 0, q is 1 and Y₃ is selected from the group consisting of saturated or unsaturated, linear or branched C₂-C₄₀ hydrocarbon chains, optionally substituted, polyethylene glycol, polypropylene glycol, pHPMA, PLGA, polymers of alkyl diamines and combinations thereof.

In some embodiments, said chemically-modified cysteine residue of formula (I) is such that:
Y is a spacer of formula (II) wherein, q is 1, m is 0 or 1, p is 0 or 1, Y₁ and Y₂ are as defined in claim 2, and Y₃ is selected from the group consisting of saturated or unsaturated, linear or branched C₂-C₄₀ hydrocarbon chains, optionally substituted, polyethylene glycol, polypropylene glycol, pHPMA, PLGA, polymers of alkyl diamines and combinations thereof, and/or
M comprises, or consists of, a click-chemistry group, an oligonucleotide, a targeting agent such as a cell-type ligand preferably selected from a mono- or a polysaccharide, a hormone, including a steroid hormone, a peptide such as RGD peptide, a muscle targeting peptide (MTP) or Angiopep-2, a protein or a fragment thereof, a membrane receptor or a fragment thereof, an aptamer, an antibody including heavy-chain antibody, and fragments thereof such as Fab, Fab′, and VHH, a ScFv, a spiegelmer, and a peptide aptamer, vitamins and drugs such as CB1 and/or CB2 ligands.

In some embodiments, said chemically-modified cysteine residue of formula (I) is such that:
Y is a spacer of formula (II) wherein, q is 1, m is 0 or 1, p is 0 or 1, Y₁ and Y₂ are as defined in claim 2, and Y₃ is selected from the group consisting of linear or branched C₂-C₂₀ alkyl chains, polyethylene glycol, polypropylene glycol, pHPMA, PLGA, polymer of alkyl diamine and combinations thereof, said polymers having from 2 to 20 monomers, and/or
“M” comprises, or consists of, a targeting agent preferably a cell-type specific ligand derived from a protein selected from transferrin, Epidermal Growth Factor (EGF), and basic Fibroblast Growth Factor βFGF, a mono- or a polysaccharide comprising one or several galactose, mannose, N-acetylgalactosamine residues, bridge GalNac, or mannose-6-phosphate, sialic acid and derivatives thereof (such as Neu5Ac, Neu5Acα2-6Gal, and Neu5Acα2-8Neu5Ac), a muscle targeting peptide (MTP) selected from SEQ ID NO:1 to SEQ ID NO:7, and vitamins such as folic acid.

In some embodiments, said chemically-modified cysteine residue of formula (I) is such that:
M is a cell-type specific ligand for specifically targeting hepatocytes and comprises at least one moiety of formula (III):

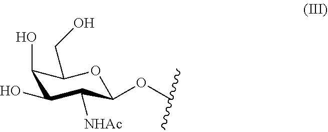

Y is a polyethylene glycol chain comprising from 2 to 10 monomers.

In some embodiments, said at least one chemically-modified cysteine in the capsid, is of formula (Ic-1):

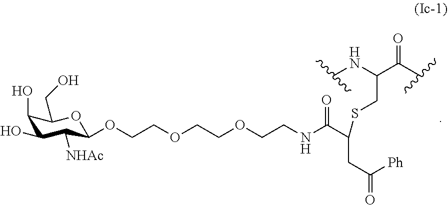

In some embodiments, the AAV of the present invention further has at least one additional chemically modified amino acid residue in the capsid, which is different from a cysteine residue, said amino acid residue preferably bearing:
a modified amino group of formula (V):

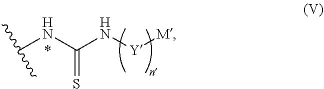

wherein:
   N* being the nitrogen of the amino group of an amino acid residue, e.g. of a lysine residue or arginine residue,
   Y′, n′ and M′ have respectively the same definition as Y, n, and M in formula (I) as defined herein; or
   a modified tyrosyl residue of formula (VI):

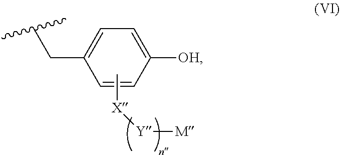

wherein:
   X″ is —N═N— or

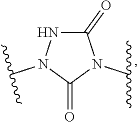 [SMILES: *n1[nH]c(=O)n(*)c1=O]

Y″, n″ and M″ have respectively the same definition as Y, n, and M in formula (I) as defined herein.

In particular, the AAV may be a recombinant AAV, preferably selected from AAV having a wildtype capsid, naturally-occurring serotype AAV, variant AAV, pseudotype AAV, AAV with hybrid, and self-complementary AAV.

Another object of the present invention is a method for chemically-modifying the capsid of an AAV, more precisely for chemically modifying at least one cysteine residue in the capsid of an AAV, which comprises incubating said AAV with a chemical reagent bearing a reactive group selected from a maleimide, a vinyl sulfonamide and a 3-(carboxy derivative)acrylamide in conditions conducive for reacting said reactive group with a cysteine residue present in the capsid of the AAV so as to form a covalent bound.

In some embodiments, said method comprises incubating the AAV with a chemical reagent of formula (VIIc):

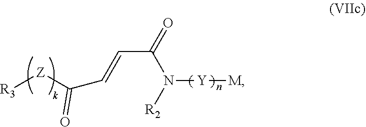

so as to obtain at least one chemically-modified cysteine residue in the capsid of formula (Ic):

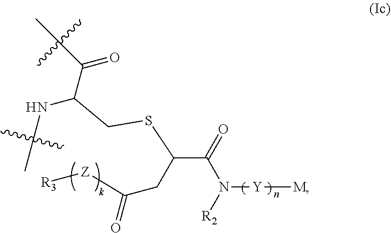

wherein:
   Y is a spacer, n is 0 or 1, M is a functional moiety,
   Z is —O—, —S—, or —N(R₄)—,
   k is 0 or 1, and
   R₂, R₃ and R₄ are each independently chosen from a hydrogen atom, an alkyl group, an aryl group, and a heteroaryl group, said group being optionally substituted.

In some embodiments, the incubation step is performed at a pH from 5.0 to 11, preferably from 6.0 to 10.0 such as from 7.0 to 8.0 or from 8.0 to 10.0.

In some embodiments, Y, in said method, is a spacer of formula (II):

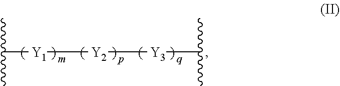

in which m is 0, p is 0, q is 1 and Y₃ is selected from the group consisting of saturated or unsaturated, linear or branched C₂-C₄₀ hydrocarbon chains, optionally substituted, polyethylene glycol, polypropylene glycol, pHPMA, PLGA, polymers of alkyl diamines and combinations thereof, and wherein M is a click-chemistry group, a steric shielding agent, a labelling agent, a targeting agent such as a cell-type specific ligand or a drug moiety.

The present invention also relates to an AAV obtainable by the method as defined herein.

The present invention further relates to a gene vector which is an AAV as defined herein and which comprises a transgene sequence in its viral genome. Preferably, such a gene vector is used in gene therapy, to deliver the transgene sequence which encodes for a therapeutic protein, in a cell, in vivo or ex vivo.

Another object of the present invention is a pharmaceutical composition comprising an AAV as defined herein or an AAV obtainable by the method as defined herein, and at least one pharmaceutically acceptable excipient.

The present invention also relates to an AAV as defined herein, an AAV obtainable by the method as defined herein, or a pharmaceutical composition as defined herein, for use as a diagnostic agent or as a drug, preferably in gene therapy. In some embodiments, said AAV or said pharmaceutical composition are used as a diagnostic agent in vivo or as a drug, preferably in gene therapy, in vivo.

In some other embodiments, the AAV or the pharmaceutical composition containing it, are used as a diagnostic agent in vitro or as a gene vector ex vivo or in vitro.

### FIGURES

FIG. 1A shows the general strategy of coupling according to the invention. GalNAc-benzoyl-acrylamide (L) corresponds to a ligand of the invention while GalNac control (c) corresponds to a control compound (which is devoid of any reactive group specific to thiol function of cysteine).

FIG. 1B shows the relative positions of conserved cysteine residues on VP subunits of various naturally occurring AAV serotypes. The numbering is based on residues within the AAV2 VP1 subunit (From Pulichera et al. PloSone, 2012, 7(2):e32163). The peptide regions shown in FIG. 1B correspond to SEQ ID NO:12-29 of the sequence listing.

FIG. 2A shows the dot blot analysis using immunostaining with A20 antibody which recognizes the assembled AAV2 capsid for AAV2 and AAV2 after being incubated with the ligand of invention (L) or the control compound (C).

FIG. 2B shows the western blots analysis using immunostaining with polyclonal antibodies directed against capsid proteins (VP), for AAV2 and AAV2 after being which recognizes the assembled AAV2 capsid for AAV2 and AAV2 after being incubated with the ligand of invention (L) or the control compound (C). Capsid protein molecular weight is indicated at the right of the image according to a protein ladder.

FIG. 3A, 3B and 3C show some results of LC-MS/MS for enzymatically digested AAV2 and AAV9 capsids chemically modified with (L) or not.

FIG. 3A shows that a peptide fragment present in VP subunits of AAV2 and AAV9 and devoid of cysteine did not undergo chemical modification as no shift in LC peak and no change in mass was observed. FIGS. 3B and 3C showed that cysteines at position 289 in AAV9 and AAV2 VPs were successfully chemically modified as evidenced by the shift in LC peak and MS; The same result was observed for cysteine at position 394 (data not shown). The LC-MS/MS analysis clearly demonstrates an effective and highly specifically coupling of the ligand of the invention with cysteine residues.

FIGS. 4A and 4B: show examples of “M” moieties according to the invention.

FIGS. 5A and 5B show the dot blot analysis using immunostaining with A20 antibody and using staining with soybean lecithin for (i) AAV2 particles, (ii) AAV2 particles which underwent coupling procedure with ligand L, and (iii) with AAV2 which underwent coupling procedure with ligand L2 at pH 7.3 (FIG. 5A) or at pH 9.3 (FIG. 5B).

### DETAILED DESCRIPTION OF THE INVENTION

The methods described in the prior art for chemically modifying AAV capsids target amino acid residues bearing amino groups such as arginine and lysine or tyrosine.

The Inventors identified cysteine amino acids as potential residues of interest to be chemically modified in AAV capsid.

Cysteine is a sparse residue in VP1, VP2 and VP3 of AAV capsids. For instance, there are five conserved cysteine residues per VP protein of AAV2 and thus at most 300 cysteine residues for a complete AAV2 capsid. These five conserved residues are located in positions 230, 289, 361, 394 and 482 in VP1 of AAV2. Structural analysis of AAV serotype 2 reveals that Cys289 and Cys361 are located adjacent to each other within each monomer, while Cys230 and Cys394 are located on opposite edges of each subunit and juxtaposed at the pentamer interface. The Cys482 residue is located at the base of a surface loop within the trimer region.

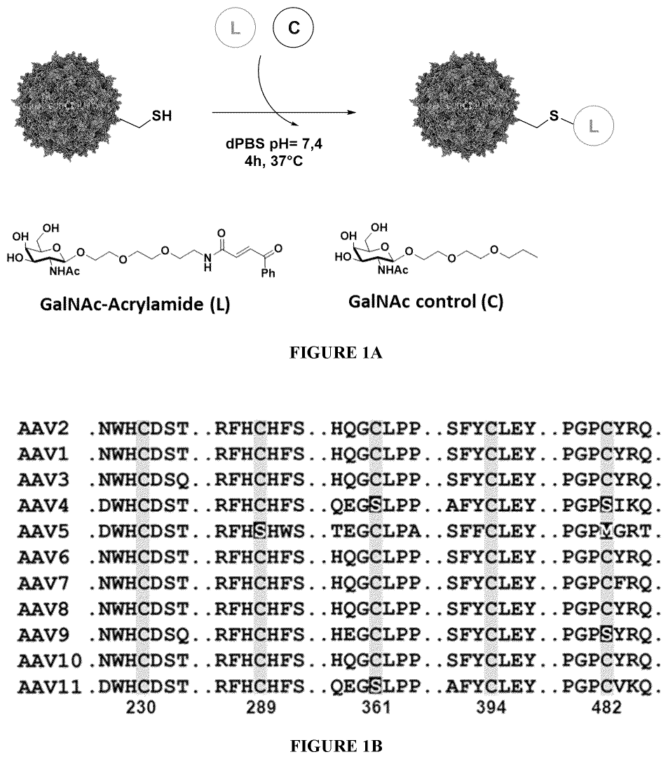

As shown in the partial alignments of VP subunits of naturally occurring AAV serotypes with the AAV2 VP1 subunits (see FIG. 1), cysteine at positions 230 and 394 are fully conserved while C289S, C361S and C482S/C482M changes are noted in AAV4, AAV5 and AAV9. For instance, the positions of the cysteine residues in AAV9 V1 subunit are Cys230, Cys291, Cys363 and Cys396, the numbering referring to the amino acid numbering in the AAV2 VP1 subunit. Thus, there are f at most 240 cysteine residues for a complete AAV9 capsid.

Little is known about the biological function of cysteine. Pulichera et al. (PloSone, 2012, 7(2):e32163) show that none of the five cysteine residues appear to be involved in disulfide bond formation and that Cys230 and Cys394 may play a role in transduction and proteasome degradation.

Thus, the chemical modification of cysteine, in particular Cys230 and Cys394, may enable to modulate the transduction efficacy and the proteasome-degradation of AVV2. There is no specific description of the impact of the cysteine residue for other AAV serotypes, such as AAV9.

Besides, due to the few numbers of cysteine residues available for chemical modifications, the Inventors are of the opinion that the chemical coupling of cysteine enables to better control the number of ligands coupled to the AAV (as compared to other more abundant residues such as tyrosine and arginine) and to introduce larger ligands without impairing the functionality of the AAV.

To the knowledge of the Inventors, the chemical coupling of ligands on naturally-occurring cysteine in AAV capsid has never been described or suggested in the prior art.

In that context, the Inventors conceived a method for chemically modifying cysteine residues present on the surface of AAV capsid. This method relies on the specific reaction of a benzoyl acrylamide derivative with the thiol function present in the side chain of cysteine residue.

The inventors prepared a benzoyl acrylamide ligand bearing a sugar moiety for chemically modifying the capsids of AAV vectors on naturally-occurring cysteine residues.

Accordingly, as a proof of concept, the Inventors showed that N-acetyl galactosamine moiety can be covalently immobilized on the surface of AAV capsid by incubating AAV particles with a benzoyl acrylamide bearing N-acetyl galactosamine (compound L) in aqueous buffer at neutral pH, and at room temperature. Of note the coupling neither impaired the overall integrity of the capsid as evidenced by dot blot with A20 antibody immunostaining nor that of individual capsid proteins, namely VP1, VP2 and VP3 as evidenced by western blot with anti-VP polyclonal antibodies. Such results suggest that the infectiosity of AAV is maintained after coupling.

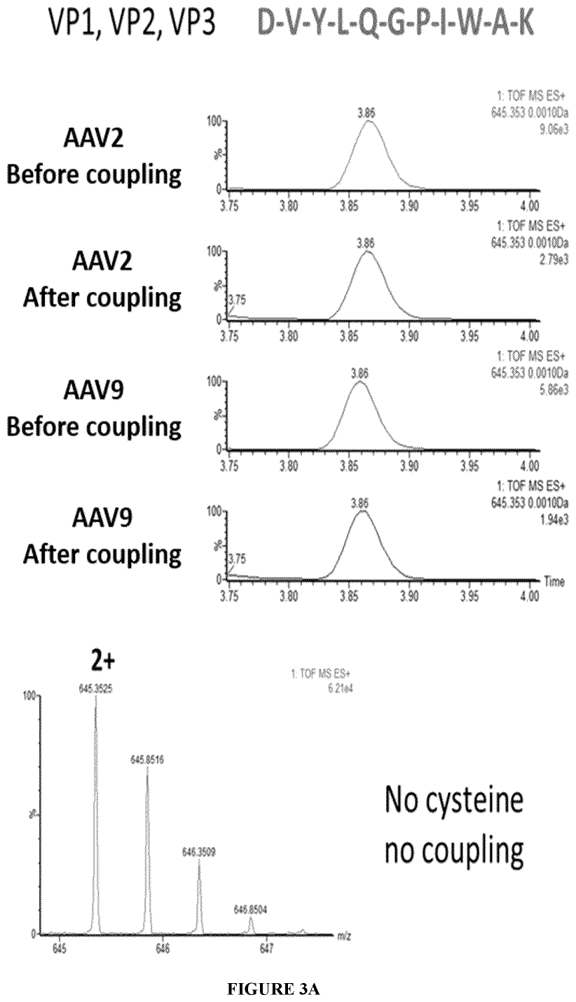

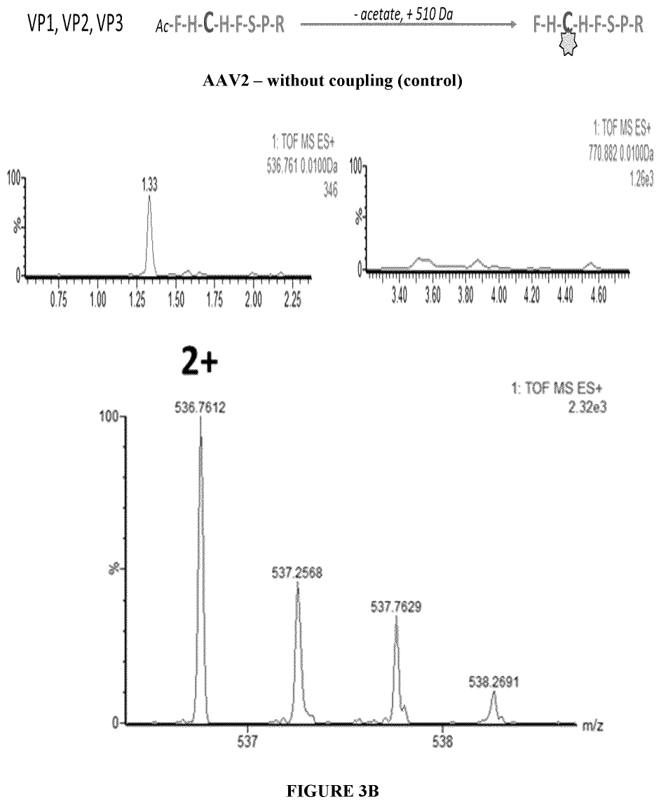

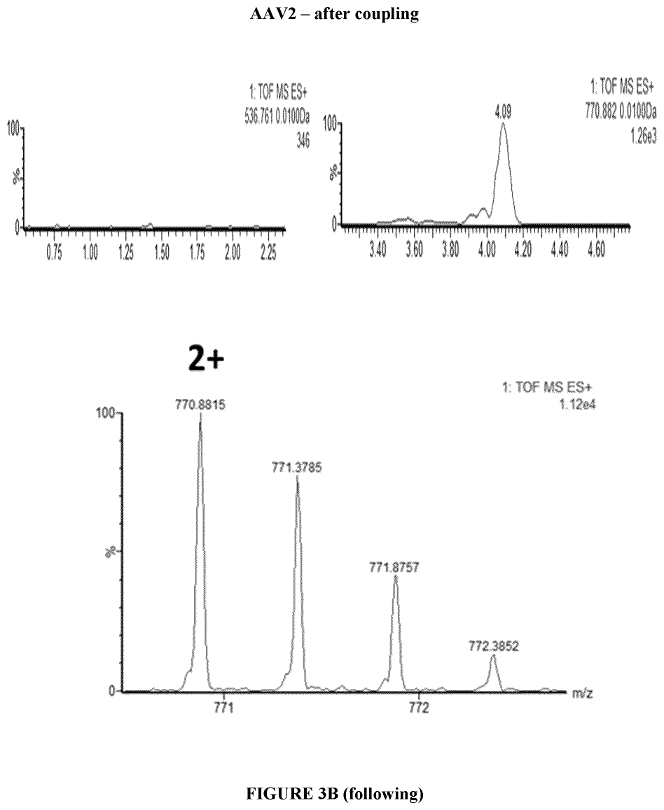

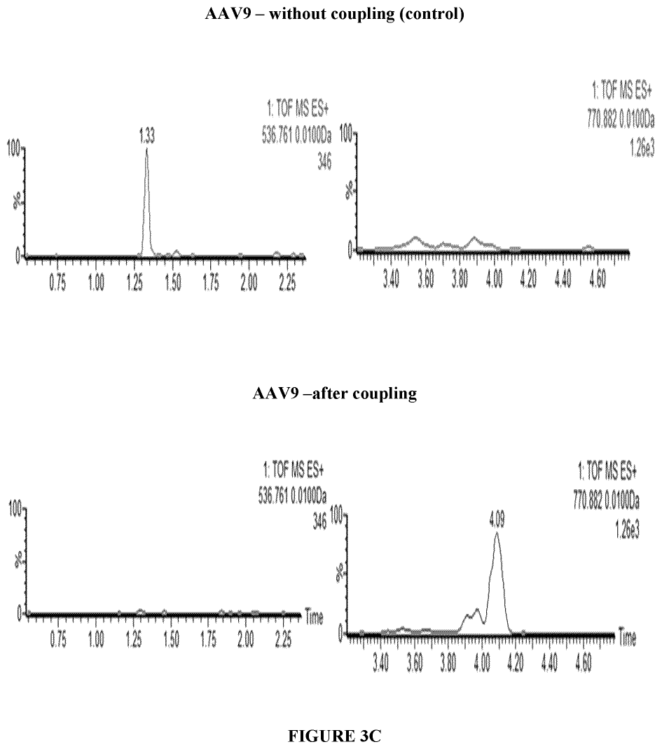

Further, the LC-MS/MS peptide analysis after enzymatic digestion of AAV vectors showed that the chemical coupling of the invention is highly effective since the digested peptide candidates bearing a cysteine showed coupling (one mass peak detected only): cysteines at position corresponding to cysteine 289 and 394 in AAV2 VP1 were fully chemically coupled in VP1, VP2 and VP3 as evidenced by the LC peak shift in terms of time retention and detected mass before and after chemical coupling. Of note, no peak shift was observed for peptide candidates devoid of any cysteine, which shows that the method is highly selective since no cross-reaction with residues other than cysteine was observed (see FIGS. 3A-3C).

Besides, the Inventors demonstrated that the AAV2 chemically modified with the benzoyl acrylamide ligand efficiently transduced HeLa cells and thus remained infectious in an order of magnitude similar to the non-chemically modified AAV2.

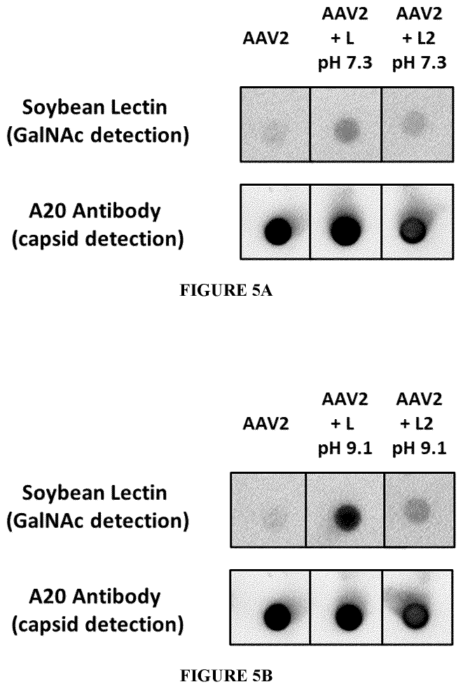

The covalent coupling of AAV2 by ligand L was effective in both acid and basic pH (Example 5). However, the Inventors showed that the proportion of chemically-modified cysteine residues can be tuned by modulating the pH of the coupling step: As evidenced by the staining in dot blots analysis (FIGS. 5A and 5B), higher the pH, higher the coupling.

In other words, with the benzoyl acrylamide ligand, it is possible to control the proportion of chemically-modified cysteines in the capsid through the pH of coupling. Such a property can be of high interest depending on the functional moiety to be covalently coupled on the surface of the AAV.

Without to be bound by any theory, the Inventors are of the opinion that the results obtained with benzoyl acrylamide ligand may be extrapolated to vinyl sulfonamide group. In a certain extent, ligands bearing maleimide could be also contemplated to chemically modify cysteine residues present in AAV capsids. However, the maleimide ligand L2 appears to be less effective and results in coupling function less stable than that obtained with acrylamide ligands.

Accordingly, the invention relates to an Adeno-Associated Virus (AAV) having at least one chemically-modified cysteine residue in its capsid.

In a particular aspect, the Invention relates to an Adeno-Associated Virus (AAV) wherein the capsid comprises a functional moiety as described herein, said functional moiety being covalently attached to a cysteine residue of the capsid. In other words, said cysteine residue is chemically modified.

The chemically-modified cysteine residue present in the AAV capsid typically results from the reaction of a cysteine in the capsid with a functional moiety bearing a reactive group specific to thiol. Such a reactive group encompasses benzoyl acrylamide group, a maleimide group or a vinyl sulfonamide group.

Preferably, the cysteine residue to be chemically modified is a naturally-occurring residue in the capsid, i.e. the cysteine has not been introduced by mutagenesis.

Accordingly, in a particular aspect, the chemically modified adeno-Associated Virus (AAV) comprises a functional moiety, for instance a ligand, covalently linked to the thiol group of a cysteine residue in its capsid via one of the following moieties:

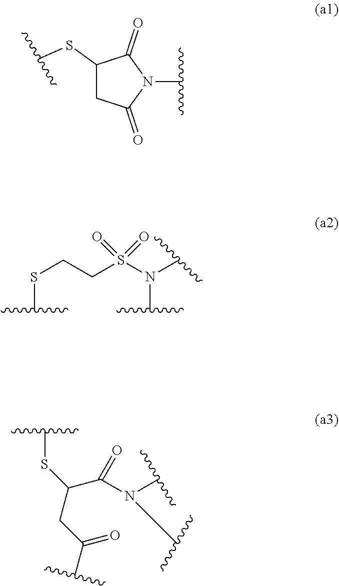 [SMILES: *SC1CC(=O)N(*)C1=O ; *SCCS(=O)(=O)N(*)* ; *SC(CC(*)=O)C(=O)N(*)*]

the “S” denoting the atom of the initial thiol group in the cysteine residue.

The Invention also relates to a method for preparing a chemically-modified AAV comprising contacting the AAV with a functional moiety bearing a reactive group specific to thiol such as benzoyl acrylamide group, a maleimide group or a vinyl sulfonamide group, in condition enabling the reaction of the thiol function of a cysteine residue present in the AAV capsid with said reactive group so as to covalently link said chemical moiety to the AAV.

The invention also relates to the uses of the resulting AAV, in particular in gene therapy.

The Invention is described in more details hereunder:

### General Definitions

The term “Cₓ-C_{y}” in which x and y are integers, as used in the present disclosure, means that the corresponding hydrocarbon chain comprises from x to y carbon atoms. If, for example, the term C₁-C₆ is used, it means that the corresponding hydrocarbon chain may comprise from 1 to 6 carbon atoms, especially 1, 2, 3, 4, 5 or 6 carbon atoms. If, for example, the term C₂-C₅ is used, it means that the corresponding hydrocarbon chain may comprise from 2 to 5 carbon atoms, especially 2, 3, 4, or 5 carbon atoms.

As used herein, the term “alkyl” refers to a saturated, linear or branched aliphatic group. A preferred alkyl is a “C₁-C₆ alkyl”, which refers to an alkyl having 1 to 6 carbon atoms. Examples of alkyl (or C₁-C₆ alkyl) include, but are not limited to methyl, ethyl, propyl, isopropyl, butyl, isobutyl, pentyl, hexyl.

As used herein, the term “alkene” or “alkenyl” refers to an unsaturated, linear or branched aliphatic group, having at least one carbon-carbon double bond. A preferred alkene is a “C₂-C₆ alkene”, which refers to an alkene having 2 to 6 carbon atoms. Examples of alkene (or C₂-C₆ alkene) include for instance ethenyl, propenyl, butenyl, pentenyl, or hexenyl, preferably ethenyl (—CH═CH₂).

As used herein, the term “alkyne” or “alkynyl” refers to an unsaturated, linear or branched aliphatic group, having at least one carbon-carbon triple bond. A preferred alkyne is “C₂-C₆ alkyne”, which refers to an alkyne having 2 to 6 carbon atoms. Examples of alkyne (or C₂-C₆ alkyne) include for instance ethynyl, propynyl, butynyl, pentynyl, or hexynyl, preferably ethynyl (—C≡CH).

As used herein, the term “alkoxy” refers to an alkyl as defined herein, attached to the remainder of the molecule via an ether bond (—O—). In other words, an alkoxy can be written “—O-alkyl”. A preferred alkoxy is a C₁-C₆ alkoxy, which has 1 to 6 carbon atoms. Examples of alkoxy (or C₁-C₆ alkoxy) include for instance, methoxy, ethoxy, propoxy, isopropoxy, butoxy, pentoxy, hexyloxy.

As used herein, the term “alkylthio” refers to an alkyl as defined herein, attached to the remainder of the molecule via a thioether bond (—S—). In other words, an alkylthio can be written “—S-alkyl”. A preferred alkylthio is a C₁-C₆ alkylthio, which has 1 to 6 carbon atoms. Examples of alkylthio (or C₁-C₆ alkylthio) include for instance, methylthio, ethylthio, propylthio, isopropylthio, butylthio, pentylthio, hexylthio.

As used herein, the term “alkylamino” refers to an alkyl as defined herein, attached to the remainder of the molecule via an amino bond (—NH—). In other words, an alkylamino can be written “—NH-alkyl”. A preferred alkylamino is a C₁-C₆ alkylamino, which has 1 to 6 carbon atoms. Examples of alkylamino (or C₁-C₆ alkylamino) include for instance, methylamino, ethylamino, propylamino, isopropylamino, butylamino, pentylamino, hexylamino.

As used herein, the term “hydrocarbon cycle” refers to a saturated or unsaturated, aliphatic or aromatic, mono-, bi- or tri-cyclic group. The hydrocarbon cycle may be in particular a cycloalkyl, a cycloalkenyl, or an aryl.

As used herein, the term “cycloalkyl” refers to a saturated mono-, bi- or tri-cyclic aliphatic group. It also includes fused, bridged, or spiro-connected cycloalkyl groups. The term “C₃-C₆ cycloalkyl” refers to a cycloalkyl having 3 to 6 carbon atoms. Examples of cycloalkyl (or C₃-C₆ cycloalkyl) include, but are not limited to cyclopropyl, cyclobutyl, cyclopentyl, cyclohexyl.

The term “cycloalkyl” may also refer to a bridged carbocyclyl such as bicyclo[2,2,1]heptanyl, bicyclo[2,2,2]octanyl, or adamantyl.

As used herein, the term “cycloalkenyl” refers to an unsaturated mono-, bi- or tri-cyclic aliphatic group, comprising at least one carbon-carbon double bond. It also includes fused, bridged, or spiro-connected cycloalkenyl groups. The term “C₃-C₆ cycloalkenyl” refers to a cycloalkenyl having 3 to 6 carbon atoms. Examples of cycloalkenyl (or C₃-C₆ cycloalkenyl) include, but are not limited to cyclopentenyl, and cyclohexenyl.

As used herein, the term “heterocycle” corresponds to a saturated or unsaturated, aliphatic or aromatic, mono-, bi- or tri-cyclic group, comprising at least one heteroatom such as nitrogen, oxygen, or sulphur atom. In the case of a bi- or tricycle, wherein the cycles can be fused, bridged or have a spiro configuration. Advantageously, the heterocycle comprises between 3 and 6 ring atoms, wherein at least one of the ring atoms is a heteroatom such as nitrogen, oxygen or sulphur atom. In some embodiments, the “heterocycle” is a heterocycloalkyl, a heterocycloalkenyl, or a heteroaryl.

As used herein, the term “heterocycloalkyl” corresponds to a cycloalkyl group as above defined in which at least one carbon atom has been replaced with a heteroatom such as nitrogen, oxygen, or sulphur atom.

As used herein, the term “heterocycloalkenyl” corresponds to a cycloalkenyl group as above defined in which at least one carbon atom has been replaced with a heteroatom such as nitrogen, oxygen, or sulphur atom.

Examples of heterocycles, which are heterocycloalkyl or heterocycloalkenyl, include, but are not limited to, aziridinyl, azepanyl, diazepanyl, dioxolanyl, benzo [1,3]dioxolyl, azetidinyl, oxetanyl, pyrazolinyl, pyranyl, thiomorpholinyl, pyrazolidinyl, piperidyl, piperazinyl, 1,4-dioxanyl, imidazolinyl, pyrrolinyl, pyrrolidinyl, piperidinyl, imidazolidinyl, morpholinyl, 1,4-dithianyl, pyrrolidinyl, pyrimidinyl, oxozolinyl, oxazolidinyl, isoxazolinyl, isoxazolidinyl, thiooxetanyl, thiopyranyl, thiomorpholinyl, thiazolinyl, thiazolidinyl, isothiazolinyl, isothiazolidinyl, dihydropyranyl, dihydrofuranyl, dihydrothiopyranyl, dihydrothiophenyl, dihydropiperidinyl, tetrahydropiperidinyl, tetrahydrothiopyranyl, tetrahydropyranyl, tetrahydrofuranyl, and tetrahydrothiophenyl.

As used herein, the term “aryl” refers to an aromatic ring system, which preferably has 6-14 atoms, having at least one ring having a conjugated pi electron system and which optionally may be substituted. An “aryl” may contain more than one aromatic ring such as fused ring systems or an aryl group substituted with another aryl group. Aryl encompass, without being limited to, phenyl, anthracenyl, naphthyl, indenyl, divalent biphenyl. “Heteroaryl” refers to a heteroaryl group. “Heteroaryl” refers to a chemical group, preferably having 5-14 ring atoms, wherein 1 to 4 heteroatoms are ring atoms in the aromatic ring and the remainder of the ring atoms being carbon atoms. Suitable heteroatoms include oxygen, sulfur, nitrogen, phosphorus, and selenium. Examples of heterocycles, which are heteroaryl groups, include furanyl, thienyl, pyridyl, pyrrolyl, N-alkyl pyrrolyl, pyridyl-N-oxide, pyrimidyl, pyrazinyl, imidazolyl, benzimidazolyl, benzofuranyl, benzothiophenyl, quinazolinyl, and quinolinyl.

Examples of bicyclic heteroaryl groups encompass, without being limited to bicyclic heteroaryl groups that may be mentioned include 1H-indazolyl, benzo[1,2,3]thiadiazolyl, benzo[1,2,5]thiadiazolyl, benzothiophenyl, imidazo[1,2-a]pyridyl, quinolinyl, indolyl and isoquinolinyl groups.

As used herein, the term “alkanoyl” refers to an alkyl as defined herein, attached to the remainder of the molecule via an oxo group (—C(O)—). In other words, an alkanoyl can be written “—C(O)-alkyl”. A preferred alkanoyl is a C₁-C₆ alkanoyl, which has an alkyl chain of 1 to 6 carbon atoms. Examples of alkanoyl (or C₁-C₆ alkanoyl) include for instance, methanoyl, ethanoyl, propanoyl, isopropanoyl, butanoyl, pentanoyl, hexanoyl.

As used herein, the term “acyl amino” refers to a group of formula R—C(O)—NH— wherein R is a hydrocarbon group such as C₁-C₆ alkyl, a C₃-C₁₂ cycloalkyl or an aryl. A preferred acylamino is a C₁-C₆ acyl amino, which has a hydrocarbon chain of 1 to 6 carbon atoms.

As used herein, the term “ester” or “carboxy ester” refers to a —C(O)OR‘ or R’C(O)O— group, wherein R′ is any hydrocarbon group, such as a C₁-C₆ alkyl, a C₃-C₁₂ cycloalkyl or an aryl. A preferred ester is a C₁-C₆ ester, which has a hydrocarbon chain of 1 to 6 carbon atoms.

As used herein, an “alkoxycarbonyloxy” refers to a R″-C(O)—O— group where R″ is an alkoxy.

As used herein, the term “alkylene” refers to a divalent alkyl group, wherein “alkyl” is as defined herein. A preferred alkylene is a “(C₁-C₆)alkylene”, which has 1 to 6 carbon atoms. A “(C₁-C₆)alkylene” can in particular refer to a group of formula —(CH₂)_{q}— where q is an integer from 1 to 6. “(C₁-C₆)alkylene” includes for instance methylene, ethylene, propylene, butylene, isobutylene, pentylene, isopentylene, or hexylene.

As used herein, the term “arylene” refers to a divalent aryl group, wherein “aryl” is as defined herein. A preferred arylene is an arylene having 6 to 14 ring atoms. Arylene includes for instance phenylene, anthracenylene, naphthylene, indenylene, divalent biphenylene, preferably phenylene. A preferred phenylene is a para-phenylene, namely a phenylene which is attached to the rest of the molecule in two positions in para.

As used herein, the term “heteroarylene” refers to a divalent heteroaryl group, wherein “heteroaryl” is as defined herein. A preferred heteroarylene is an heteroarylene having 5 to 14 ring atoms. Heteroarylene includes for instance furanylene, thienylene, pyridylene, pyrrolylene, N-alkyl pyrrolylene, pyridylene-N-oxide, pyrimidylene, pyrazinylene, imidazolylene, benzimidazolylene, benzofuranylene, benzothiophenylene, quinazolinylene, and quinolinylene.

As used herein, the term “halogen” includes chlorine, fluorine, iodine, bromine, preferably chlorine or fluorine.

As used herein, the term “aminoalkyl” refers to an alkyl as defined above, substituted by one or more (preferably one) amino (—NH₂) group.

As used herein, the term “alkylaminoalkyl” refers to an alkyl as defined above, substituted by one or more (preferably one) alkylamino group as defined above.

As used herein, the term “hydroxyalkyl” refers to an alkyl as defined above, substituted by one or more (preferably one) hydroxy (˜OH) group.

As used herein, the term “alkoxyalkyl” refers to an alkyl as defined above, substituted by one or more alkoxy as defined above.

As used herein, the term “haloalkyl” refers to an alkyl as defined above, substituted by one or more halogen atoms.

“Substituted” or “optionally substituted” includes groups substituted by one or several substituents, typically 1, 2, 3, 4, 5 or 6 substituents. For instance, the substituents may be independently selected from C₁-C₆ alkyl, aryl, C₃-C₆ cycloalkyl, C₃-C₆ cycloalkenyl, C₂-C₆ heterocycle, C₁-C₆ alkoxy, C₁-C₆ alkylamino, C₁-C₆ aminoalkyl-, C₁-C₆ alkylaminoalkyl-, —N₃, —NH₂, —F, —I, —Br, —C₁, —CN, C₁-C₆ alkanoyl, C₁-C₆ carboxy esters, C₁-C₆ acylamino, —COOH,—CONH₂,—NO₂,—SO₃H, C₁-C₆ hydroxyalkyl, C₁-C₆ haloalkyl, C₁-C₆ alkylthio, C₂-C₁₀ alkoxyalkyl, C₂-C₆ alkoxycarbonyloxy, —CN,—CF₃ and C₂-C₆ alkoxyalkyl.

Preferred substituents are halogens, —OH, NH₂, NO₂, C₁-C₃ alkyl, C₁-C₃ alkoxy, C₁-C₃ hydroxyalkyl, and C₁-C₃ haloalkyl.

The phrase “optionally substituted” can be replaced with the phrase “substituted or unsubstituted” throughout this application.

### Chemically-Modified AAV of the Invention

Accordingly, the invention relates to an Adeno-Associated Virus (AAV) having at least one chemically-modified cysteine residue in its capsid.

As used herein, an Adeno-Associated Virus (AAV) refers to a small, nonenveloped virus of the dependoparvovirus family having a single-stranded linear DNA genome of about 5 kb long. Wild-type AAV has two major open reading frames (ORFs) flanked by two inverted terminal repeats (ITRs). The 5′ and 3′ ORFs encode replication and capsid proteins, respectively. The ITR contains 145 nucleotides and serves as the AAV genome replication origin and packaging signal. In recombinant AAV, viral ORFs are replaced by the exogenous gene expression cassette, while the replication and capsid proteins are provided in trans.

Accordingly, in the context of the Invention, a recombinant AAV refers to an AAV wherein an exogenous nucleic acid sequence e.g. a transgene sequence has been introduced in the viral genome. Said exogenous nucleic acid sequence may be of any type and is selected in view of the intended use of the AAV. For instance, said nucleic acid may comprises any RNA or DNA sequence.

In preferred embodiments, the AAV of the invention is a recombinant AAV. Typically, said recombinant AAV is to be used as a gene vector for in vivo or in vitro applications that means that the AAV of the invention is a recombinant AAV vector. For review concerning AAV as vector in gene therapy, one can refer to Naso et al., Biodrugs, 2017, 31:317-334, the content of which being incorporated herein by reference.

For illustration only, a recombinant AAV for use as vector in gene therapy may comprise an exogenous gene expression cassette replacing the viral ORFs and placed between the two ITRs.

Said cassette may comprise a promoter, the gene of interest and a terminator. The promoter and the gene of interest are selected depending on the targeted tissue/organ and the condition to treat. As another example, the recombinant AAV for use in gene therapy may comprise a DNA template for homologous recombination in cells. Such a recombinant AAV can be used in combination with gene editing tools, for promoting homologous recombination in targeted cells, in vivo, in vitro or ex vivo. The gene editing tools can be of any type, and encompass, without being limited to, CRISPR/Cas9, Zinc Finger Nuclease, meganuclease as well as RNA and DNA encoding said proteins.

In the context of the invention, the term “AAV” include all types of AAV, including wild-type AAV and recombinant or variant AAV. AAV variants encompass, without being limited to, AAV having a mutated or a synthetic capsid such as AAV with hybrid capsid, pseudotype AAV as well as self-complementary AAV (scAAV).

The capsid of a wildtype AAV is composed of three overlapping capsid proteins called viral protein 1 (VP1), VP2, and VP3. Genetic engineering of the capsid refers to amino acid modifications of said capsid protein(s), e.g. in their hypervariable loops.

As used herein, “an AAV having a genetically engineered capsid” or “An AAV having a mutated capsid” refers to an AAV wherein one or several amino acid modifications has(ve) been introduced in at least one capsid protein (namely VP1, and/or VP2 and/or VP3) as compared to the wild-type version of said capsid protein.

As used herein, “an amino acid modification” encompass the insertion, deletion or substitution of one or more (e.g. 1, 2, 3, 4, 5, 6, 7, 9, 10, 15, 20, 30, 40, 50, or 100) amino acids.

In some embodiments, the capsid of the AAV is devoid of any tetracysteine moiety. In particular, the capsid of the AVV has not been mutated or engineered so as to introduce a tetracysteine moiety. In particular, the AAV of the Invention is not an AAV8 in which a tetracysteine moiety has been introduced at a position selected from the group consisting of 34 of VP1, position 138 of VP1 or VP2 or at positions 583 or 589 of VP1, VP2 or VP3, and combinations thereof, the amino acid position referring to the amino acid numbering in AAV8 VP1.

In another embodiments, the AAV has a genetically mutated capsid, wherein the mutation (s) has(ve) not been performed on cysteine residues or have not resulted in the insertion of a tetracysteine moiety. In other words, the AAV of the invention may have a wildtype capsid or may have a mutated capsid wherein the naturally occurring cysteine residues are conserved. In a particular embodiment, the AAV of the invention is selected from wild-type AAV and recombinant or variant AAV with a wildtype AAV capsid.

As used herein, “a chemically-modified cysteine residue” means that at least one cysteine present in the capsid of the virus has been chemically modified by covalent coupling of a chemical entity, typically by the covalent coupling of a said chemical entity on the phenyl ring of the cysteine. Said cysteine is typically a surface exposed residue present in VP1, VP2 or VP3. A surface exposed cysteine means that the cysteine is reachable for covalent coupling. Such cysteine residues can be identified by molecular modelling of the capsid proteins or that of the whole capsid itself. There are 5 conserved cysteine residues in VP1, VP2 and VP3. The conserved cysteine residues are at position 230, 289, 361, 394 and 482 in AVV2 VP1 subunit. These residues are also conserved in AAV2 VP2 and VP3 subunits. The amino acid positions of said cysteine residues are 93, 152, 224, 257 and 345 in AAV2 VP2 subunit and 28, 87, 159, 192, and 280 in AAV2 VP3 subunit.

As mentioned above, cysteine in VP are well conserved among AAV serotype.

For instance, for AAV9, the cysteine residues are at position 230, 291, 363 and 396 in VP1 subunit, at position 93, 154, 226 and 259 in VP2 subunit and at position 28, 89, 161 and 194 in VP3 subunit. These cysteine residues in AAV9 VPs are at position corresponding to 230, 289, 361, and 394 of AAV2 VP1 subunit.

Thus, for sake of clarity, otherwise indicated, the cysteine position indicated for a given VP protein is provided by reference to the amino acid position numbering in AAV2 VP1. The correspondence can be made by performing partial sequence alignment between the VP protein of interest and the AAV2 VP1, e.g. as shown in FIG. 1B.

The capsids are composed of total 60 copies of the viral protein subunits VP1, VP2 and VP3 in the ratio 1:1:10. Accordingly, the capsid comprises at most 300 and 240 cysteine residues for AAV2 and AAV9 respectively. At least, cysteine residues on positions 289 and 394 for AAV2 were shown to be chemically modified for AAV2 and 291 and 396 for AAV9.

As used herein, “at least one chemically-modified cysteine residue” encompasses at least 1, 2, 3, 4, 5, 6, 7, 8, 9 10, 20, 30, 40, 50, 60, 70, 80, 90, 100, 110, 120 or more chemically-modified cysteine residue(s).

In some embodiments, the chemically-modified AAV of the invention comprises several chemically modified cysteine residues in its capsid.

Said chemically-modified cysteine may be present on VP1, and/or VP2 and/or VP3. The chemically-modified cysteine(s) may be present at position 230, 289, 361, 394 and/or 482 in VP subunit(s), said amino acid positions referring to amino acid numbering in AAV2 VP1.

In some embodiments, the chemically-modified AAV of the invention comprises chemically modified cysteine in VP1 and/or VP2 and/or VP3, at position 394, said amino acid position referring to the amino acid numbering in AAV2 VP1.

In some additional embodiments, the chemically-modified AAV of the invention comprises chemically modified cysteine(s) in VP1 and/or VP2 and/or VP3, at position 289, said amino acid position referring to the amino acid numbering in AAV2 VP1.

In some further embodiments, the chemically-modified AAV of the invention is characterized in that cysteines at position 289 and 394 in VP subunits are chemically modified with the ligand of the invention, the amino acid position referring to the amino acid numbering in AAV2 VP1.

The AAV may be of any serotype. In some particular embodiments, the chemically-modified AAV of the invention is of AAV2 serotype and comprises at least one chemically modified cysteine residue at position 289 and 394 of VP1, and/or VP2 and/or VP3, said positions referring to the amino acid numbering in VP1 for AAV2.

In some other embodiments, the chemically-modified AAV of the invention is of AAV9 serotype and comprises at least one chemically modified cysteine residue at position 291 and 396 of VP1, and/or VP2 and/or VP3, said positions referring to the amino acid numbering in VP1 for AAV9.

In some embodiments at least 1%, 5%, 10%, 20%, 30%, 40%, 50%, 60%, 70%, 80%, 90%, 95% and even 100% of the surface exposed cysteine residues of the capsid are chemically modified.

In some embodiments, at least 30%, preferably at least 50% of the cysteine residues present in the capsid, namely in VP1, VP2 and VP3 protein, are chemically modified.

In particular embodiment, at least 50%, e.g. at least 60%, 70%, 80% or 90% of the cysteine at a position 289 and 394 in VP subunits are chemically modified, the amino acid position referring to the amino acid numbering in AAV2 VP1.

In the context of the invention, the AAV may be either an AAV with wildtype capsid or an AAV having a mutated and/or a synthetic capsid.

In some embodiments, the AAV of the invention is a recombinant AAV with a wildtype capsid.

In other embodiment, the AAV is a recombinant AAV having a mutated capsid, namely one or more amino acid modifications in at least one capsid protein as compared to the corresponding parent capsid protein.

In a particular embodiment, the AAV is a recombinant AAV having a mutated capsid, wherein the amino acid modification(s) is/are not concerned with any cysteine residues present in capsid proteins.

In another embodiment, the cysteine residues in the AAV capsid are wild-type, namely naturally-occurring cysteine residues

In another embodiment, the AAV capsid does not comprise any mutation introducing a cysteine moiety, in particular a tetra-cysteine moiety.

They are various serotypes of AAV which can be either wildtype or synthetic. All serotypes are contemplated in the framework of the invention.

A “serotype” is traditionally defined on the basis of a lack of cross-reactivity between antibodies to one virus as compared to another virus. Such cross-reactivity differences are usually due to differences in capsid protein sequences/antigenic determinants (e.g., due to VP1, VP2, and/or VP3 sequence differences of AAV serotypes). AAV includes various naturally and synthetic (e.g. hybrid, chimera or shuffled serotypes) serotypes.

Such non-limiting serotypes include AAV-1, -2, -3, -4, -5, -6, -7, -8, -9, -10 (such as -cy10 or -rh10), -11, -rh74 or engineered AAV capsid variants such as AAV-2i8, AAV2G9, -LK3, -DJ, and -Anc80. In the context of the invention, synthetic serotypes also include pseudotyped AAV, namely AAV resulting from the mixing of a capsid and genome from different viral serotypes, such as AAV2/5, AAV2/7, and AAV2/8 as well as AAV with hybrid capsids derived from multiple different serotypes such as AAV-DJ, which contains a hybrid capsid derived from eight serotypes.

Synthetic serotypes also encompass specific variants wherein a new glycan binding site is introduced into the AAV capsid are in particular described in WO2014144229 (disclosing in particular the AAV2G9 serotype). Other AAV serotypes include those disclosed in EP2292779, and EP1310571. In addition, other AAV serotypes include those obtained by shuffling, as described in Koerber et al. (Molecular Therapy (2008), 16(10), 1703-1709), peptide insertion (e.g. Deverman et al., Nat Biotechnol (2016), 34(2), 204-209), or rational capsid design (reviewed in Büning et al., Curr Opin Pharmacol (2015), 24, 94-104).

In some embodiments, the AAV is selected from naturally-occurring serotypes, preferably from the group consisting of AAV-2, AAV-3b, AAV-5, AAV-8, AAV-9 and AAVrh10, more preferably AAV-2. For instance, the AAV of the invention may be of AAV-2 or AAV-9 serotype.

The AAV can target a large variety of cells, tissues and organs. Examples of cells targeted by AAV encompasses, but are not limited to, hepatocytes; cells of the retina; i.e. photoreceptors, retinal pigmented epithelium (RPE); muscle cells, i.e. myoblasts, satellite cells; cells of the central nervous system (CNS), i.e. neurons, glial; cells of the heart; cells of the peripheral nervous system (PNS); osteoblasts; tumor cells, blood cells such as lymphocytes, hematopoietic cells including hematopoietic stem cells, induced pluripotent stem cells (iPS) and the like.

Examples of tissues and organs which can be targeted by AAV include liver, muscle, cardiac muscle, smooth muscle, brain, bone, connective tissue, heart, kidney, lung, lymph node, mammary gland, myelin, prostate, testes, thymus, thyroid, trachea, and the like. Preferred cell types are hepatocytes, retinal cells, muscle cells, cells of the CNS, cells of the PNS and hematopoietic cells. Preferred tissue and organs are liver, muscle, heart, eye, and brain.

The tropism of AAV can vary depending on their serotype. For instance, AAV-2 can be used to transduce the central nervous system (CNS), kidney, and photoreceptor cells while AAV-8 is effective for transducing the CNS, heart, liver, photoreceptor cells, retinal pigment epithelium (RPE) and skeletal muscle.

The AAV can be produced by any methods known in the art, such as transient transfection in cell lines of interest e.g. in HEK293 cells as described in the Example section. To that matter one can refer to Naso et al., Biodrugs, 2017, 31:317-334 which provide a review on AAV as vectors in gene therapy, and describe the traditional methods for producing AAV at the industrial scale.

The AAV of the invention may have other amino acid(s) of the capsid which has been chemically modified. For instance, the AAV may comprise one or several amino groups of the capsid which have been modified by the method disclosed in WO2017/212019, namely by reacting said amino group(s) in the capsid with a ligand bearing an isothiocyanate reactive groups. Alternatively or additionally, the AAV of the invention may have one or several arginine residues of the capsid modified by glycation, e.g. by reaction with methylglyoxal as described in Horowitz (supra). Alternatively or additionally, the AAV of the invention may comprise one or several tyrosyl residues of the capsid which have been modified by the method disclosed in WO 2021/005210, namely by reacting said tyrosyl residue in the capsid with a ligand bearing an aryl diazonium or 4-phenyl-1,2,4-triazole-3,5-dione (PTAD) reactive groups.

Typically, the at least one chemically-modified cysteine residue in the capsid is of formula (I):

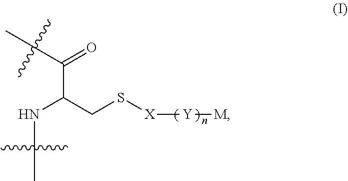

wherein:
   X is selected from the group consisting of:

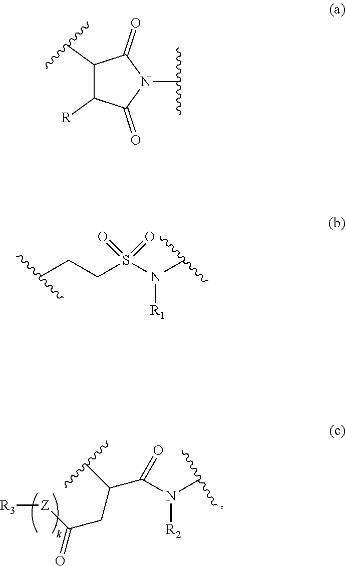

wherein
   Z is —O—, —S—, or —N(R₄)—,
   R₁, R₂, R₃, and R₄ are each independently chosen from a hydrogen atom, an alkyl group, an aryl group, and a heteroaryl group, said group being optionally substituted,
   k is 0 or 1,
   R is a hydrogen, a halogen, an alkyl group, an aryl group, a heteroaryl group, an alkoxy group, said group being optionally substituted,
   Y is a spacer,
   n is 0 or 1, and
   M is a functional moiety.

As mentioned in the above definitions, in some embodiments:
alkyl group is preferably a C₁-C₆ alkyl,
aryl group preferably is preferably an aryl group having from 6 to 14 atom rings,
heteroaryl group is preferably an aryl group having from 5 to 14 atom rings, and
alkoxy group is preferably a C₁-C₆ alkoxy group.

In the formulae described in the present application (such as in formula (I)), the following moiety represents a cysteine within a protein of the capsid (i.e. VP1, VP2, or VP3):

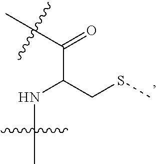

wherein

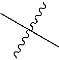

represents a bond by which the cysteine is attached to the rest of the protein.
“n is 1” means that the spacer Y is present. “n is 0” means that the spacer Y is absent.
   X
X is of formula (a), (b), or (c) as described above. Preferably, X is of formula (b) or of formula (c). More preferably, X is of formula (c).

In formula (a), R is preferably a hydrogen atom, a halogen, or a C₁-C₆ alkoxy, more preferably a hydrogen atom.

In formula (b), R₁ is preferably a C₁-C₆ alkyl, an aryl or a heteroaryl. For instance, R₁ may be selected from C₁-C₆ alkyl, aryl group comprising from 6 to 14 ring atoms, such as a phenyl and heteroaryl group comprising from 5 to 14 ring atoms. In preferred embodiments, R₁ is a C₁-C₆ alkyl, and even more preferably a C₁-C₃ alkyl such as a methyl.

In formula (c):
R₂ is hydrogen atom, an alkyl group, an aryl group, or a heteroaryl, preferably a C₁-C₃ alkyl or a hydrogen, more preferably a hydrogen.
R₃ is preferably an aryl or heteroaryl group (such as a phenyl), said group being optionally substituted. For instance, said aryl or heteroaryl group may be substituted by 1 to 3 substituents preferably chosen from halogens, —OH, NH₂, NO₂, C₁-C₃ alkyl, C₁-C₃ alkoxy, C₁-C₃ hydroxyalkyl, and C₁-C₃ haloalkyl. In a particular embodiment, R₃ is selected from an aryl group comprising from 6 to 14 ring atoms or heteroaryl group comprising from 5 to 14 ring atoms, said aryl or heteroaryl group being optionally substituted. For instance, said aryl or heteroaryl group may be substituted by 1 to 3 substituents preferably chosen from halogens, —OH, NH₂, NO₂, C₁-C₃ alkyl, C₁-C₃ alkoxy, C₁-C₃ hydroxyalkyl, and C₁-C₃ haloalkyl. In some preferred embodiments, R₃ is a phenyl group optionally substituted by one or three substituents selected from —OH, NH₂, NO₂, C₁-C₃ alkyl, C₁-C₃ alkoxy, C₁-C₃ hydroxyalkyl, and C₁-C₃ haloalkyl. The substituent(s) can be at position ortho, meta or para, preferably at position ortho or para.
Preferably, R₄ is a hydrogen or a C₁-C₃ alkyl, more preferably H or CH₃.

In a particular embodiment, k is 1. In a preferred embodiment, k is 0, which means that Z is absent.

In a particular embodiment, R₂ is a C₁-C₃ alkyl or a hydrogen, R₃ is a phenyl group optionally substituted by one or three substituents selected from —OH, NH₂, NO₂, C₁-C₃ alkyl, C₁-C₃ alkoxy, C₁-C₃ hydroxyalkyl, and C₁-C₃ haloalkyl, k is 0 or 1 (preferably, k is 0), and Z, if present, is —O—, —S—, —N(CH₃)—, or —NH—.

In a more particular embodiment, R₂ is H and R₃ is a phenyl, unsubstituted or substituted, preferably an unsubstituted phenyl.

In a preferred embodiment, R₂ is H, R₃ is a phenyl (unsubstituted or substituted, preferably an unsubstituted phenyl), and k is 0.
Spacer Y
   Y is a spacer that links X and the functional moiety M. Y may be present (when n is 1) or absent (when n is 0). When Y is absent, X and M are directly linked to each other.

Y may be any chemical chain which can comprise heteroatoms as well as cyclic moieties such as cycloalkyl, cycloalkenyl, heterocycloalkyl, or aromatic groups including heteroaryl. Y may comprise up to 1000 carbon atoms and even more. The length and the chemical nature of the spacer may be optimized depending on the functional moiety “M” which is intended to be coupled with the cysteine residues and the biological effect which is sought. Indeed, further to its linking function, Y may be used to refine the properties of the functional moiety “M”. For instance, Y may decrease the steric hindrance of M with respect to the capsid, improve the accessibility of M for binding with a biological entity of interest, improve the binding of M with an entity of interest and/or increase the solubility of M.

In some embodiments, Y is a chemical chain group comprising from 2 to 1000 carbon atoms, preferably from 2 to 500 carbon atoms, from 2 to 300 carbon atoms, e.g. from 2 to 100 carbon atoms, 2 to 40 carbon atoms, from 4 to 30 carbon atoms or from 4 to 20 carbon atoms.

In a particular embodiment, Y is a spacer of formula (II):

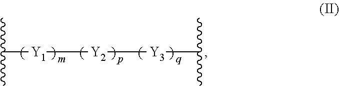

wherein:
   m, p and q are each independently 0 or 1,
   Y₁ is selected from the group consisting of an alkylene group, an arylene group, a heteroarylene group, said group being optionally substituted, preferably a phenylene group,
   Y₂ is —C(═O)—NH, —C(═O)—O, —C(═O)—O—C(═O)—, O—(C═O)—, NH—C(═O)—, NH—C(═O)—NH, —O—C═O—O—, O, NH, —NH(C═S)—, or —(C═S)—NH—, preferably —(C═O)—NH—, and
   Y₃ is selected from the group consisting of polymers including homopolymers, copolymers and block polymers, peptides, oligosaccharides, saturated or unsaturated, branched or linear hydrocarbon chains, optionally interrupted by one or several heteroatoms and/or by a group chosen from —C(═O)—NH, —C(═O)—O, —C(═O)—O—C(═O)—, O—(C═O)—, NH—C(═O)—, NH—C(═O)—NH, —O—C(═O)—O—, —NH(C═S)—, or —(C═S)—NH-, and/or by one or more C₃-C₆ hydrocarbon cycle or a C₂-C₆ heterocycle, optionally having at least one of its extremity an heteroatom, and optionally substituted by one or several substituents, and combinations thereof.

In such embodiment, said at least one chemically-modified cysteine residue in the capsid can typically be represented by the following formula (I-II):

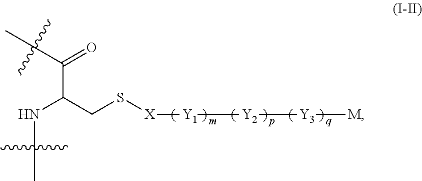

wherein X, Y₁, Y₂, Y₃, M, m, p, and q are as defined herein.

In a particular embodiment, m is 0, p is 0 and q is 1. In such embodiment, the spacer Y is Y₃.

In another particular embodiment, m is 1, p is 1, and q is 1.

In some embodiments, Y₁ is an unsubstituted or substituted C₁-C₆ alkylene. For instance, said C₁-C₆ alkylene may be substituted by 1 to 3 substituents, which may be independently selected from halogens, —OH, NH₂, NO₂, C₁-C₃ alkyl, C₁-C₃ alkoxy, C₁-C₃ hydroxyalkyl, and C₁-C₃ haloalkyl.

In some embodiments, Y₁ is selected from arylene and heteroarylene comprising from 5 to 14 ring atoms, e.g. from 6 to 10 ring atoms, said arylene or heteroarylene being optionally substituted. For instance, said arylene or heteroarylene group may comprise 1, 2 or 3 substituents independently selected from halogens, —OH, NH₂, NO₂, C₁-C₃ alkyl, C₁-C₃ alkoxy, C₁-C₃ hydroxyalkyl, and C₁-C₃ haloalkyl.

In some particular embodiments, Y₁ is selected from the group consisting of substituted or unsubstituted phenylene, pyridylene, naphthylene, and anthracenylene. Said groups may comprise from 1 to 3 substituents, preferably independently selected from halogens, —OH, NH₂, NO₂, C₁-C₃ alkyl, C₁-C₃ alkoxy, C₁-C₃ hydroxyalkyl, and C₁-C₃ haloalkyl.

Preferably, Y₁ is a phenylene, optionally substituted by 1 to 3 substituents, which may be independently selected from halogens, —OH, NH₂, NO₂, C₁-C₃ alkyl, C₁-C₃ alkoxy, C₁-C₃ hydroxyalkyl, and C₁-C₃ haloalkyl.

In an embodiment where Y₁ is a phenylene (optionally substituted by 1 to 3 substituents, which may be independently selected from halogens, —OH, NH₂, NO₂, C₁-C₃ alkyl, C₁-C₃ alkoxy, C₁-C₃ hydroxyalkyl, and C₁-C₃ haloalkyl), X and —(Y₂)ₚ—(Y₃)_{q}-M are preferably connected in para position on said phenylene.

In a particular embodiment, Y₂ is —(C═O)—NH—.

As mentioned above, Y₃ is selected from the group consisting of polymers including homopolymers, copolymers and block polymers, peptides, oligosaccharides, saturated or unsaturated, branched or linear hydrocarbon chains, optionally interrupted by one or several heteroatoms (e.g. S, O, Se, P or NH) and/or by a group chosen from —C(═O)—NH, —C(═O)—O, —C(═O)—O—C(═O)—, O—(C═O)—, NH—C(═O)—, NH—C(═O)—NH, —O—C(═O)—O—, —NH(C═S)—, or —(C=S)—NH-, and/or by one or more C₃-C₆ hydrocarbon cycle or a C₂-C₆ heterocycle, optionally having at least one of its extremity an heteroatom such as S, O and NH, and optionally substituted by one or several substituents such as hydroxyl, halogens, C₁-C₃ alkoxy, —CN, -CF₃, or C₁-C₃ alkyl, and combinations thereof.

For instance, Y₃ may be selected from the group consisting of polymers including homopolymers, copolymers and block polymers, peptides, oligosaccharides, saturated or unsaturated, branched or linear hydrocarbon chains.

As used herein, “combinations” means that Y₃ may comprise several hydrocarbon chains, oligomer chains or polymeric chains (e.g. 2, 3, 4, 5 or 6) linked by any appropriate group, such as —O—, —S—, —NHC(O)—, —OC(O)—, —C(O)—O—C(O)—, —NH—, —NH—CO—NH—, —O—CO—NH—, NH—(CS)—NH—, NH—CS—, phosphodiester or phosphorothioate groups.

In some embodiments, Y₃ may be selected from the group consisting of polyethers such as polyethylene glycol (PEG) and polypropylene glycol, polyvinyl alcohol (PVA), polyesters such as polylactate, polyacrylate, polymethacrylate, polysilicone, polyamide such as polycaprolactone and poly(N-(2-hydroxypropyl)methacrylamide) (pHPMA), poly(D,L-lactic-co-glycolic acid) (PLGA), polymers of alkyl diamines, unsaturated or saturated, branched or unbranched, hydrocarbon chains optionally having an heteroatom such as 0, NH and S on at least one end, and combinations thereof.

As used herein, alkyl diamine refers to NH₂-(CH₂)ᵣ-NH₂ with r is an integer from 2 to 20, for instance from 2 to 10 such as 2, 3, 4, and 5. A polymer of alkyl diamines (also known as polyamines) refers to a compound of formula NH₂-[(CH₂)ᵣ-NH]ₜ—H with r being as defined above and t is an integer of at least 2, for example of at least 3, 4, 5, 10 or more. Polymers of alkyl diamines of interest are, for instance, spermidine, and spermine. For instance, Y₃ can comprise at least one polyethylene glycol moiety comprising from 2 to 40 monomers, e.g. from 2 to 10 or 2 to 6 monomers. For illustration only, Y₃ may comprise from 2 to 10 triethyleneglycol blocks linked together by linkers. As another example, Y₃ may be a C₁₂ hydrophilic triethylene glycol ethylamine derivative. Alternatively, Y₃ may be a saturated or unsaturated C₂-C₄₀ hydrocarbon chain, in particular a C₁₀-C₂₀ alkyl chain or a C₂-C₁₀ alkyl chain such as a C₆ alkyl chain. The alkyl chain may have a group such as NH, S or 0 on at least one end.

As further examples, Y₃ may be selected from spermidine, putrescine, spermine and combinations thereof.

In a particular embodiment, Y₃ is selected from the group consisting of saturated or unsaturated, linear or branched C₂-C₄₀ hydrocarbon chains, optionally substituted, polyethylene glycol, polypropylene glycol, pHPMA, PLGA, polymers of alkyl diamines and combinations thereof.

In a particular embodiment, Y₃ is selected from the group consisting of linear or branched C₂-C₂₀ alkylene chains, polyethylene glycol, polypropylene glycol, pHPMA, PLGA, polymers of diamino alkyl and combinations thereof. Preferably said polyethylene glycol, polypropylene glycol, PLGA, pHPMA and polymer of alkyl diamines comprise from 2 to 40 monomers, preferably from 2 to 10 or from 10 to 20 monomers.

For instance, Y₃ may comprise one or several (e.g. 2, 3, 4 or 5) triethylene glycol blocks.

### X+Y Moiety

In a particular embodiment, X is of formula (a), n=1, and Y is of formula (II), wherein:

m=p=q=1
Y₁ is as defined herein, preferably a C₁-C₆ alkylene group or phenylene group, more preferably a phenylene group, said group being optionally substituted by 1 to 3 substituents, which may be independently selected from halogens, —OH, NH₂, NO₂, C₁-C₃ alkyl, C₁-C₃ alkoxy, C₁-C₃ hydroxyalkyl, and C₁-C₃ haloalkyl,
Y₂ is as defined herein, preferably Y₂ is —(C═O)—NH—, and
Y₃ is as defined herein, preferably selected from the group consisting of linear or branched C₂-C₂₀ alkylene chains, polyethylene glycol, polypropylene glycol, pHPMA, PLGA, polymers of diamino alkyl and combinations thereof.

In another particular embodiment, X is of formula (b), n=1, and Y is of formula (II), wherein:

m=p=q=1
Y₁ is as defined herein, preferably a phenylene group, said group being optionally substituted by 1 to 3 substituents, which may be independently selected from halogens, —OH, NH₂, NO₂, C₁-C₃ alkyl, C₁-C₃ alkoxy, C₁-C₃ hydroxyalkyl, and C₁-C₃ haloalkyl,
Y₂ is as defined herein, preferably Y₂ is —(C═O)—NH—, and
Y₃ is as defined herein, preferably selected from the group consisting of linear or branched C₂-C₂₀ alkylene chains, polyethylene glycol, polypropylene glycol, pHPMA, PLGA, polymers of diamino alkyl and combinations thereof.

In a preferred embodiment, X is of formula (c), n=1, and Y is of formula (II), wherein:
m=0, p=0 or 1 (preferably 0), q=1, and
Y₂ is as defined herein,
Y₃ is as defined herein, preferably selected from the group consisting of linear or branched C₂-C₂₀ alkylene chains, polyethylene glycol, polypropylene glycol, pHPMA, PLGA, polymers of diamino alkyl and combinations thereof.

### Functional Moiety M

The functional moiety “M” may be of any type. “M” is typically selected depending on the biological effect which is sought when chemically modifying the capsid of the AAV.

For instance, “M” may comprise a moiety selected from a chemical reactive group such as a click-chemistry reactive group, a targeting agent, a steric shielding agent, a labelling agent, an oligonucleotide, or a drug. “M” may be also a (nano)-particle, including a magnetic (nano-) particle and a quantum dot. For instance, M may be an iron, stain, silicium, gold or carbon (nano)-particle.

In some embodiments, “M” comprises, or consists of, a labeling agent, e.g. a fluorescent dye such as fluorescein, rhodamine, boron-dipyrromethene (Bodipy) dyes, and alexa fluor, or a radionuclide.

In other embodiments, “M” comprises, or consists of, a steric shielding agent, e.g. an agent able to mask certain epitopes of the capsid, whereby avoiding the binding of neutralizing antibodies.

For instance, “M” may be a polyethylene glycol (PEG), pHPMA or a polysaccharide.

In a specific embodiment, “M” comprises, or consists of, a steric shielding agent able to mask cysteine residues, whereby proteasome-degradation of the AAV in cellulo is avoided.

In another embodiment, “M” may be an oligonucleotide such as messenger RNA (mRNa) or antisense oligonucleotides such as small interferent RNA (siRNA), shRNA, snoRNA and meroduplex (mdRNA).

In some embodiments, M comprises, or consists of, a targeting agent, namely a ligand enabling to target a specific organ, tissue, cell, or a protein of interest, such as a cell surface protein, receptor or oligosaccharides, e.g. a cell surface protein that is present at the surface of a particular cell line or a tumoral cell.

For instance, the targeting agent can be a cell-type specific ligand, namely a ligand enabling to target a specific type of cell.

Such a ligand may enable to modify the tropism of the AAV, namely its capacity to selectively infect and/or transduce a given cell line, tissue or organ.

For instance, “M” may be a ligand which specifically binds to a membrane biological entity (e.g. a membrane receptor) of the targeted cell. Said ligand may be, for instance, a mono- or a polysaccharide, a hormone, including a steroid hormone, a peptide such as RGD peptide, Angiopep-2, muscle targeting peptides, a protein or a fragment thereof, a membrane receptor or a fragment thereof, CB1 and CB2 ligands, an aptamer, an antibody including heavy-chain antibody, and fragments thereof such as Fab, Fab′, and VHH, a ScFv, a spiegelmer, a peptide aptamer, a small chemical molecules known to bind to the targeted biological entity and the like.

In a particular embodiment, M is an antibody including a full length antibody or an antigen-binding domain derived from an antibody.

As used herein, the term “antibody” refers to an immunoglobulin or a fragment or a derivative thereof, and encompasses any polypeptide comprising an antigen-binding domain, regardless whether it is produced in vitro or in vivo. The term includes, but is not limited to, polyclonal, monoclonal, monospecific, multispecific (e.g. bispecific), humanized, single-chain, chimeric, synthetic, recombinant, hybrid, mutated, and grafted antibodies. The term “antibody” also includes antibody fragments such as Fab, F(ab′)2, Fv, scFv, Fd, dAb, and other antibody fragments (e.g. VHH from single-chain antibody) that retain antigen-binding function, i.e., the ability to bind their target specifically. Typically, such fragments would comprise an antigen-binding domain. The terms “antigen-binding domain,” or “antigen-binding fragment,” refer to a part of an antibody molecule that comprises amino acids responsible for the specific binding between the antibody and the antigen. Where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain may comprise an antibody light chain variable region (VL) and an antibody heavy chain variable region (VH). However, it does not necessarily comprise both (see e.g. the antigen-binding domain of single chain antibodies and VHH fragments). Typically, an antigen-binding fragment or domain contains at least a portion of the variable regions (heavy and light) of the antibody sufficient to form an antigen binding site (e.g., one or more CDRs, and generally all CDRs) and thus retains the binding specificity and/or activity of the antibody.

As used herein, a “full-length antibody” (also called herein immunoglobulin of Ig) refers to a protein having the structure that constitutes the natural biological form of an antibody, including variable and constant regions. “Full length antibody” covers both monoclonal and polyclonal full-length antibodies and also encompasses wild-type full-length antibodies, chimeric full-length antibodies, humanized full-length antibodies, the list not being limitative. In most mammals, including humans and mice, the structure of full-length antibodies is generally a tetramer. Said tetramer is composed of two identical pairs of polypeptide chains, each pair having one “light” (typically having a molecular weight of about 25 kDa) and one “heavy” chain (typically having a molecular weight of about 50-70 kDa). In the case of human immunoglobulins, light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. Thus, “isotype” as used herein is meant any of the classes of immunoglobulins defined by the chemical and antigenic characteristics of their constant regions. The known human immunoglobulin isotypes are IgG1, IgG2, IgG3, IgG4, IgA1, IgA2, IgM1, IgM2, IgD, and IgE.

In some embodiments, “M” comprises, or consists of, a cell-type specific ligand derived from proteins such as transferrin, Epidermal Growth Factor (EGF), and basic Fibroblast Growth Factor βFGF.

In some other embodiments, “M” comprises, or consists of, a cell-type specific ligand derived from mono- or polysaccharides, e.g. comprising one or several galactose, mannose, mannose-6-phosphate, N-acetylgalactosamine (GalNac) and bridged GalNac and sialic acid and derivatives thereof (such as Neu5Ac, Neu5Acα2-6Gal, Neu5Acα2-8Neu5Ac). The mono- or polysaccharides can be natural or synthetic.

In another embodiment, “M” comprises, or consists of, a cell-type specific ligand derived from vitamins such as folic acid.

According to one embodiment, the cell-type specific ligand included in “M” may be derived from, or may consist in, a muscle targeting peptide (MTP). Said ligand may comprise an amino acid sequence selected from the group consisting of: ASSLNIA (SEQ ID NO: 1); WDANGKT (SEQ ID NO: 2); GETRAPL (SEQ ID NO: 3); CGHHPVYAC (SEQ ID NO: 4); HAIYPRH (SEQ ID NO: 5), cyclic CQLFPLFRC (SEQ ID NO: 6) or the sequence of SEQ ID NO:7 as shown below:

(SEQ ID NO: 7)
R-X-R-R-B-R-R-X-R-F-Q-I-L-Y-R-X-R-B-R-X-R-B

wherein X is an amino hexanoic acid residue and B is a beta-alanine residue.

As used herein, cyclic CQLFPLFRC of SEQ ID NO:6 refers to:

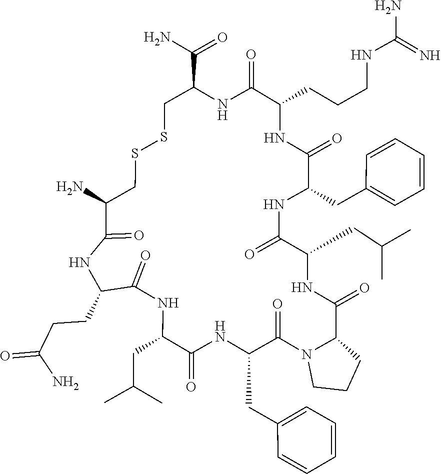 [SMILES: CC(C)C[C@@H]1NC(=O)[C@H](CCC(N)=O)NC(=O)[C@@H](N)CSSC[C@@H](C(N)=O)NC(=O)[C@H](CCCNC(=N)N)NC(=O)[C@H](Cc2ccccc2)NC(=O)[C@H](CC(C)C)NC(=O)[C@@H]2CCCN2C(=O)[C@H](Cc2ccccc2)NC1=O]

In certain embodiments, “M” is a cancer cell targeting peptide and comprises a peptide such as RGD, including cyclic RGD.

In some other embodiments, M is a cell type targeting ligand selected from antibodies and fragments thereof.

When “M” comprises a peptide moiety, such as a muscle targeting peptide (MTP), said peptide moiety may comprise a chemical modification at its N-terminus or C-terminus. For instance, the N-terminus of the peptide moiety can be acylated or coupled to a moiety such as —C(═O)—(PEG moiety)-NH₂.

In another embodiments, “M” comprises, or consists of, a cell-type specific ligand derived from small molecules or hormones such as naproxen, ibuprofen, cholesterol, progesterone or estradiol.

In an additional embodiment, “M” comprises, or consists of, a CB1 and/or a CB2 ligand, for instance:

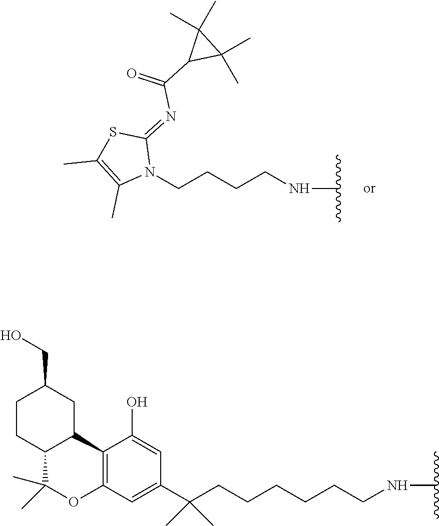

Galactose-derived ligands, which are recognized by asialoglycoprotein receptor (ASPGPr), can be used to specifically target hepatocytes. Accordingly, in some embodiments “M” is a ligand for specifically targeting hepatocytes and comprises at least one moiety of formula (IIIa), (IIIb) or (IIIc):

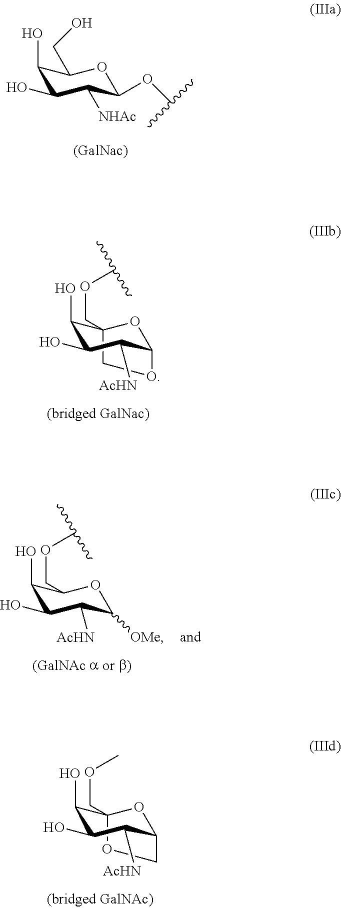

In some other embodiments, “M” is a ligand for targeting muscle cells, in particular skeletal muscle cells and comprises at least one mannose-6-phosphate moiety:

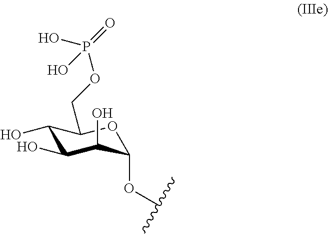

In some other embodiments, “M” is a ligand for photoreceptors or neuronal cells and comprises at least one mannose moiety of formula (IIIf):

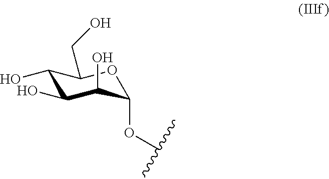

In some other embodiments, “M” is a ligand for Siglecs proteins (sialic-acid-bending immunoglobulin-like lectins). In some embodiments, M is sialic acid moiety or a derivative thereof. As used herein, “sialic acid moiety and derivatives thereof” include a moiety comprising one or more N-acylated neuraminic acid units and optionally one or more other saccharide units such as a galactose moiety.

More specifically, M may be a sialic acid moiety or a derivative thereof, said moiety comprising or consisting of at least one moiety of formula (IIIg):

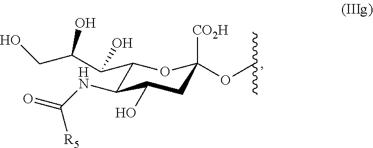

wherein R₅ is an alkyl, an aryl, a heteroaryl, haloalkyl (such as —CH₂-Hal, where Hal is a halogen), —OR₆, —NR₇R₈,—SR₉, —CH₂OR₁₀, —CH₂NR₁₁R₁₂, or —CH₂SR₁₃
where R₆, R₇, R₈, R₉, R₁₀, R₁₁, R₁₂, and R₁₃ are each independently chosen from a hydrogen atom, an alkyl group, an aryl group, and a heteroaryl group.

In a particular embodiment, R₅ is an alkyl, —OR₆, —CH₂OR₁₀, —CH₂-Hal, where Hal, R₆, R₁₀ are as defined herein.

In a more particular embodiment, R₅ is methyl, —CH₂OH, or —CH₂—F.

In a particular embodiment, M is chosen from a Neu5Ac, Neu5Acα2-6Gal and Neu5Acα2-8Neu5Ac moiety.

As used herein, Neu5Ac moiety refers to N-acetylneuraminic acid moiety. Neu5Ac can be represented by the following formula (IIIh):

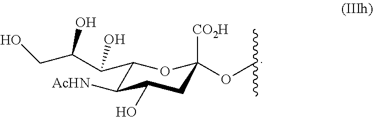

As used herein, Neu5Acα2-6Gal moiety refers to a moiety consisting of a N-acetylneuraminic acid unit and a galactose unit bonded by a α2-6 bond. Neu5Acα2-6Gal moiety can be represented by the following formula (IIIi):

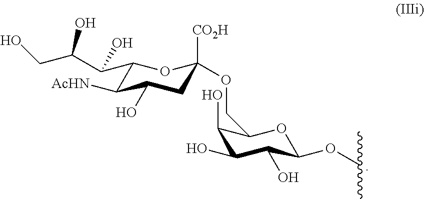

As used herein, Neu5Acα2-8Neu5Ac moiety refers to a moiety consisting of two N-acetylneuraminic acid units bonded by a α2-8 bond. Neu5Acα2-8Neu5Ac can be represented by the following formula (IIIj):

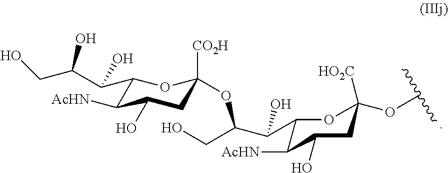

In some embodiments, “M” is multivalent, which means that it comprises at least two (e.g. 2, 3, 4, 5, or 6) ligand moieties of interest, such as cell-type specific ligands as described above. For instance, M may comprise a polyfunctional linker bearing several (e.g. at least 2, 3, 4, 5, or 6) cell-type ligands. The cell-type ligands can be the same or different.

For instance, “M” may comprise a moiety of formula (IV):

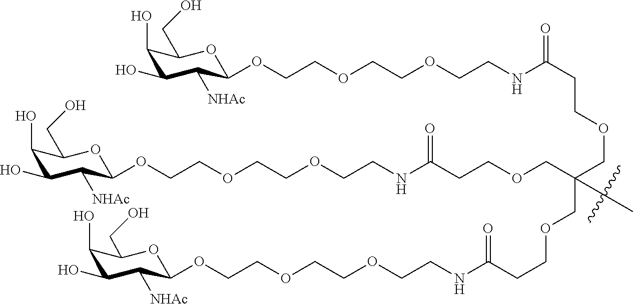

with n is a enter from 1 to 100, preferably from 1 to 20.

As another example of multivalent ligands, “M” may comprise a moiety of formula (IV) wherein the GalNac groups are replaced by mannose, phosphate-6-mannose, bridged GalNac, sialic acid or derivatives thereof (e.g. Neu5Ac, Neu5Acα2-6Gal, Neu5Acα2-8Neu5Ac for instance as shown above), CB1 and/or CB2 ligands or peptides.

In some particular embodiment, “M” may comprise both a labelling moiety such as a fluorescent label or a radionuclide and a cell-type specific ligand. For illustration only, M may be:

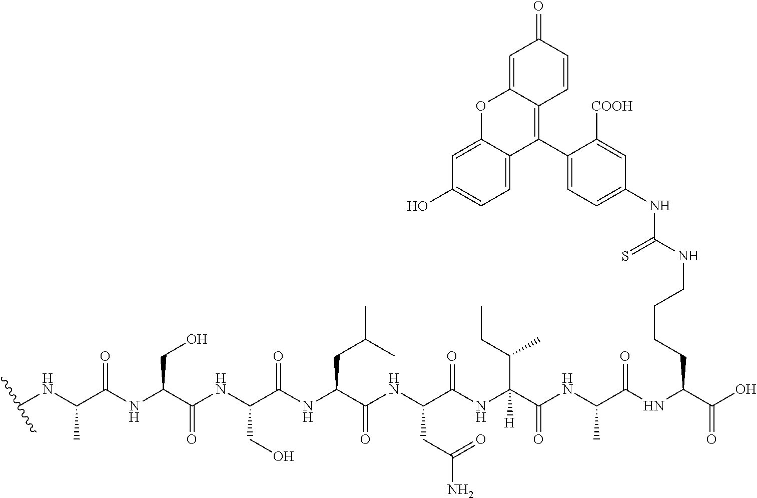 [SMILES: *N[C@@H](C)C(=O)N[C@@H](CO)C(=O)N[C@@H](CO)C(=O)N[C@@H](CC(C)C)C(=O)N[C@@H](CC(N)=O)C(=O)N[C@H](C(=O)N[C@@H](C)C(=O)N[C@@H](CCCCNC(=S)Nc1ccc(-c2c3ccc(=O)cc-3oc3cc(O)ccc23)c(C(=O)O)c1)C(=O)O)[C@@H](C)CC]

namely a muscle targeting peptide of SEQ ID NO:1 linked to K-FITC.

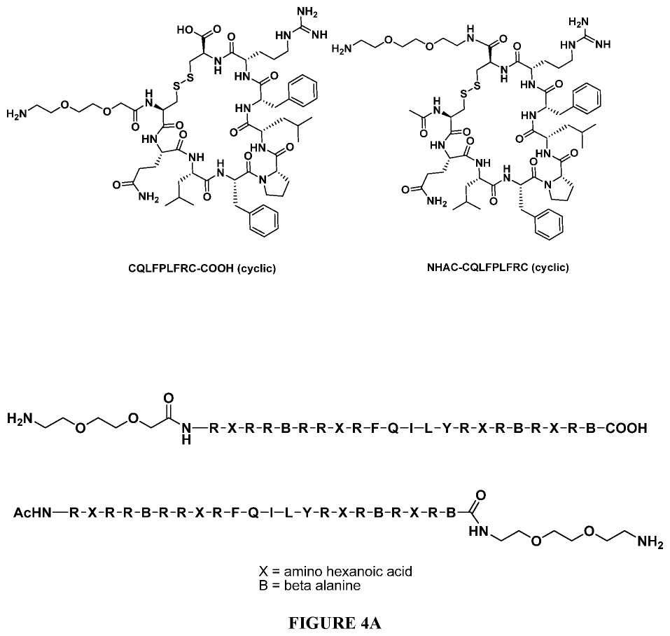

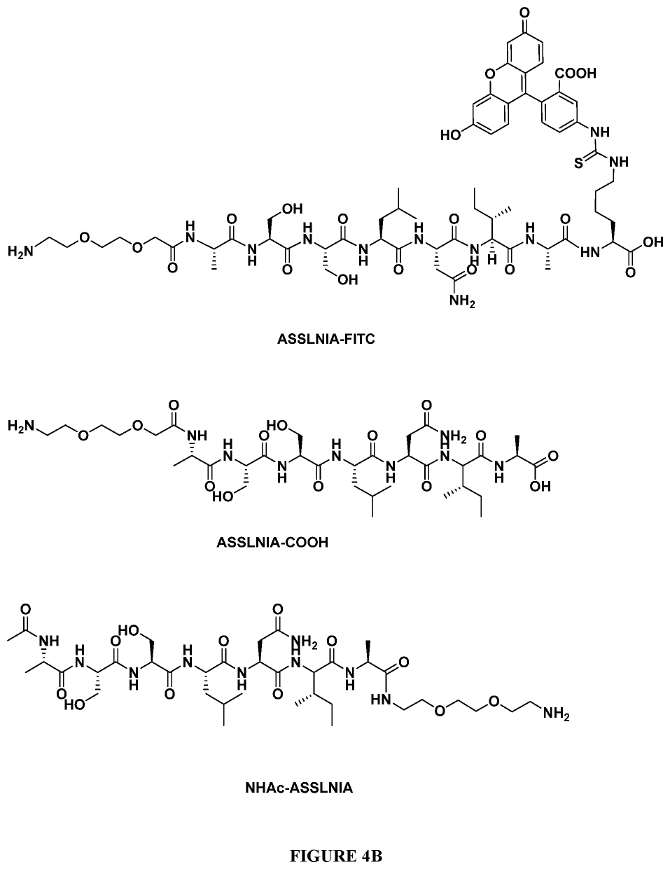

Other examples of chemical moieties which can be used as “M” moieties are provided in FIG. 4A and in FIG. 4B.

In some embodiments, M is a chemical reactive group, more preferably a “biocompatible chemical reactive group. As used herein, M can enable to create a covalent interaction between the AAV and an entity of interest, without significantly altering the functionality of the AAV (and thus in a biocompatible way). In other words, the functional moiety may comprise a chemical reactive group which can promote the formation of a covalent bond with the entity of interest. For instance, the functional moiety may comprise a chemical reactive group suitable to create a covalent bond by click-chemistry or by bioconjugation reaction. Bioconjugation reactions encompass reactions between amino acids such as lysine, cysteine or tyrosine with reactive groups as detailed in Koniev, O., Wagner, A, Chem. Soc. Rev., 44, 5495 (2015).

Preferably, M is a click-chemistry reactive group, also called hereunder a “click-chemistry group”.

As used herein, a “click-chemistry group” refers to any reactive chemical group that can be involved in a click chemistry reaction. Preferably, M is not a thiol (—SH).

“Click-reaction” or “Click-chemistry” is a concept introduced by Sharpless in 2001. “Click chemistry” generally refers to chemical reactions characterized by high yields, high chemoselectivity, which are simple to conduct and which generate inoffensive by-products.

“Click reactions” can be typically conducted in complex media with high efficiency. Click reactions are typically used to create covalent heteroatom links (C—X-C) between two entities of interest. For review about click chemistry, one can refer to Kolb et al., Angew. Chem. Int. Ed. 2001, 40, 2004-2021) and to Rudolf et al., Current opinion in Chemical Biology, 2013, 17:110-117.

Examples of click chemistry reactions include, but are not limited to, Staudinger Ligation, azido-ene or azido-alkyne click-chemistry, carbonyl condensation, sydnone-alkyne cycloaddition, tetrazole-ene reaction, nitrile oxide-ene click chemistry, nitrile imine-ene click chemistry, inverse electron demand Diels-Alder ligation, isonitrile-tetrazine click chemistry, Suzuki-Miyaura coupling, or His-tag. Preferably, the click chemistry reaction is not thiol-ene or thiol-maleimide reaction.

For instance, M may comprise, or consist of, an azido (—N₃), an alkene, an alkyne (in particular a strained alkyne, such as cyclooctyne (OCT), aryl-less cyclooctyne (ALO), monofluorocyclooctyne (MOFO), difluorocyclooctyne (DIFO), dibenzocyclooctyne (DIBO), dimethoxyazacyclooctyne (DIMAC), biarylazacyclooctynone (BARAC), bicyclononyne (BCN), tetramethylthiepinium (TMTI, TMTH), difluorobenzocyclooctyne (DIFBO), oxa-dibenzocyclooctyne (ODIBO), carboxymethylmonobenzocyclooctyne (COMBO), or benzocyclononyne), phosphine such as a triarylphosphine, aldehyde, ketone, hydrazide, oxyamine, nitrile oxide, oxime, hydroxymoyl chloride, chlororoxime, nitrile imine, hydrazone, hydrazonoyl chloride, chlorohydrazone, tetrazine, tetrazole isonitrile, aryl halide, aryl boronate, oligo-histidine, nickel-complex or nickel ligand.

In a particular embodiment, M is a click-chemistry group comprising an azido group (—N₃) or an alkyne group (such as a —C≡CH group, or a strained alkyne such as cyclooctyne (OCT), aryl-less cyclooctyne (ALO), monofluorocyclooctyne (MOFO), difluorocyclooctyne (DIFO), dibenzocyclooctyne (DIBO), dimethoxyazacyclooctyne (DIMAC), biarylazacyclooctynone (BARAC), bicyclononyne (BCN), tetramethylthiepinium (TMTI, TMTH), difluorobenzocyclooctyne (DIFBO), oxa-dibenzocyclooctyne (ODIBO), carboxymethylmonobenzocyclooctyne (COMBO), or benzocyclononyne).

As mentioned above, preferred chemically-modified AAV are those comprising at least one chemically-modified cysteine of formula (I) or (I-II), wherein X is of formula (c). Indeed, as illustrated in the Example section, acrylamide ligands enable to obtain significantly higher coupling rate than maleimide ligands and are thus more effective. Besides, the proportions of chemically-modified cysteine residues in the AAV capsid can be tuned with acrylamide ligands by modulating the pH of coupling. At last, cysteine residues modified with acrylamide ligands are expected to be more stable than those modified with maleimide ligands.

Accordingly, in a preferred aspect, the invention relates to AAV particle comprising at least one chemically-modified cysteine present in the capsid, which is of formula (Ic):

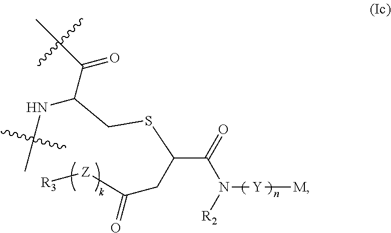

wherein Y, n, M, Z, k, R₂ and R₃ are as defined herein.

It is understood that, when Z is as defined herein, and if Z is NR₄, then R₄ is as defined herein.

More particularly, the invention relates to AAV particle comprising at least one chemically-modified cysteine present in the capsid, which is of formula (I-IIc):

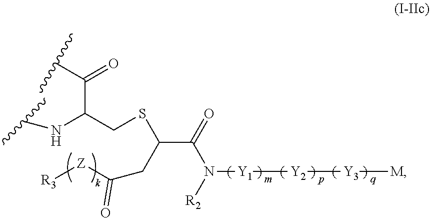

wherein Y₁, Y₂, Y₃, m, p, q, M, Z, k, R₂ and R₃ are as defined herein.

In some embodiments of the invention, the chemically-modified cysteine present in the capsid is of formula (Ic) or (I-IIc) and is further characterized by one or several of the following features:
R₂ is a hydrogen,
R₃ is an aryl or heteroaryl group, preferably a phenyl, said group being optionally substituted by 1 to 3 substituents preferably chosen from halogens, —OH, NH₂, NO₂, C₁-C₃ alkyl, C₁-C₃ alkoxy, C₁-C₃ hydroxyalkyl, and C₁-C₃ haloalkyl,
k is 0 or 1 (preferably k is 0),
Z, when present, is —O—, —S—, —N(CH₃)— or —NH—,
M is as defined above. M may be selected from a chemical reactive group such as a click-chemistry reactive group, a targeting agent, a steric shielding agent, a labelling agent, an oligonucleotide, or a drug.

In some other embodiments of the invention, the chemically-modified cysteine present in the capsid is of formula (Ic) or (I-IIc) and is further characterized by the following features:
R₂ is hydrogen atom, a C₁-C₆ alkyl group, an aryl group comprising from 6 to 14 ring atoms, or a heteroaryl group comprising from 5 to 14 ring atoms, and
R₃ is selected from an aryl group comprising from 6 to 14 ring atoms or heteroaryl group comprising from 5 to 14 ring atoms, said aryl or heteroaryl group being optionally substituted. For instance, said aryl or heteroaryl group may be substituted by 1 to 3 substituents preferably chosen from halogens, —OH, NH₂, NO₂, C₁-C₃ alkyl, C₁-C₃ alkoxy, C₁-C₃ hydroxyalkyl, and C₁-C₃ haloalkyl.

Preferably k is 0.

In some further other embodiments of the invention, the chemically-modified cysteine present in the capsid is of formula (Ic) or (I-IIc) and is further characterized by the following features:
R₂ is a C₁-C₃ alkyl or a hydrogen, preferably a hydrogen and
R₃ is a phenyl or a 6 atom ring heteroaryl, preferably a phenyl, unsubstituted or substituted by 1 to 3 substituents selected from halogens, —OH, NH₂, NO₂, C₁-C₃ alkyl, C₁-C₃ alkoxy, C₁-C₃ hydroxyalkyl, and C₁-C₃ haloalkyl.

Preferably k is 0.

In another embodiment of the invention, the chemically-modified cysteine present in the capsid is of formula (Ic) or (I-IIc) and is further characterized by the following features:
R₂ is a C₁-C₃ alkyl or a hydrogen,
R₃ is a phenyl unsubstituted or substituted with a group selected from halogens, —OH, NH₂, NO₂, C₁-C₃ alkyl, C₁-C₃ alkoxy, C₁-C₃ hydroxyalkyl, and C₁-C₃ haloalkyl, and
k is 0.

In all the herein described embodiments, M may comprise, or may consist of a cell targeting agent, preferably selected from a mono- or a polysaccharide, a hormone, including a steroid hormone, a peptide such as RGD peptide, muscle targeting peptides (MTP), or Angiopep-2, a protein or a fragment thereof, a membrane receptor or a fragment thereof, an aptamer, an antibody including heavy-chain antibody, and fragments thereof such as Fab, Fab′, and VHH, a ScFv, a spiegelmer, a peptide aptamer, a vitamin and small chemical molecules such as drugs e.g. CB1 and/or CB2 ligands. In another embodiment, M comprises, or consists of a click-chemistry reactive group e.g. comprising an azido or an alkyne, or an oligonucleotide, e.g. as defined above.

In some embodiments of the invention, the chemically-modified cysteine present in the capsid is of formula (I-IIc) and is further characterized by one or several of the following features:
m=0,
p=0,
q=1,
Y₃ is as defined herein, preferably selected from the group consisting of linear or branched C₂-C₂₀ alkylene chains, polyethylene glycol, polypropylene glycol, pHPMA, PLGA, polymers of diamino alkyl and combinations thereof,
R₂ is a hydrogen,
R₃ is an aryl or heteroaryl group, preferably a phenyl, said group being optionally substituted by 1 to 3 substituents preferably chosen from halogens, —OH, NH₂, NO₂, C₁-C₃ alkyl, C₁-C₃ alkoxy, C₁-C₃ hydroxyalkyl, and C₁-C₃ haloalkyl,
k is 0 or 1 (preferably k is 0),
Z, when present, is —O—, —S—, —N(CH₃)—, or —NH—,
M is as defined above. In particular M comprises, or consists of a click-chemistry reactive group (e.g. comprising an azido or an alkyne), an oligonucleotide, a cell-type targeting ligand, preferably selected from a mono- or a polysaccharide, a hormone, including a steroid hormone, a peptide such as RGD peptide, muscle targeting peptides (MTP), or Angiopep-2, a protein or a fragment thereof, a membrane receptor or a fragment thereof, an aptamer, an antibody including heavy-chain antibody, and fragments thereof such as Fab, Fab′, and VHH, a ScFv, a spiegelmer, a peptide aptamer, a vitamin and small chemical molecules such as drugs e.g. CB1 and/or CB2 ligands.

In some embodiments, Y, when present, is a spacer of formula (II) wherein q is 1, m is 0 or 1, p is 0 or 1, Y₁ and Y₂ are as defined above in formula (II), and Y₃ is selected from the group consisting of saturated or unsaturated, linear or branched C₂-C₄₀ hydrocarbon chains, optionally substituted, polyethylene glycol, polypropylene glycol, pHPMA, PLGA, polymers of alkyl diamines and combinations thereof and/or M comprises, or consists of, a click-chemistry group, an oligonucleotide, a cell-type targeting ligand, preferably selected from a mono- or a polysaccharide, a hormone, including a steroid hormone, a peptide such as RGD peptide, a muscle targeting peptide (MTP) or Angiopep-2, a protein or a fragment thereof, a membrane receptor or a fragment thereof, an aptamer, an antibody including heavy-chain antibody, and fragments thereof such as Fab, Fab′, and VHH, a ScFv, a spiegelmer, a peptide aptamer, vitamins and drugs such as CB1 and/or CB2 ligands.

In some other embodiments, Y, when present, is a spacer of formula (II) wherein q is 1, m is 0 or 1, p is 0 or 1, Y₁ and Y₂ are as defined above in formula (II), and Y₃ is selected from the group consisting of saturated or unsaturated, linear or branched C₂-C₄₀ hydrocarbon chains, optionally substituted, polyethylene glycol, polypropylene glycol, pHPMA, PLGA, polymers of alkyl diamines and combinations thereof and/or M comprises, or consists of, a cell-type specific ligand derived from a protein selected from transferrin, Epidermal Growth Factor (EGF), and basic Fibroblast Growth Factor βFGF, a mono- or a polysaccharide comprising one or several galactose, mannose, N-acetylgalactosamine residues, bridge GalNac, or mannose-6-phosphate, sialic acid and derivatives thereof (e.g. Neu5Ac, Neu5Acα2-6Gal, Neu5Acα2-8Neu5Ac . . . ), a MTP selected from SEQ ID NO:1 to SEQ ID NO:7, and vitamins such as folic acid.

In some embodiments, Y, when present, is a spacer of formula (II) wherein q is 1, m is 0 or 1, p is 0 or 1, Y₁ and Y₂ are as defined above in formula (II), and Y₃ is selected from the group consisting of linear or branched C₂-C₄₀ alkyl chains, polyethylene glycol, polypropylene glycol, pHPMA, PLGA, polymers of alkyl diamines and combinations thereof, wherein the polymer preferably comprises from 2 to 40 monomers and/or M comprises, or consists of, a click-chemistry group, cell-type targeting ligand, preferably selected from a mono- or a polysaccharide, a hormone, including a steroid hormone, a peptide such as RGD peptide, a muscle targeting peptide (MTP) or Angiopep-2, a protein or a fragment thereof, a membrane receptor or a fragment thereof, an aptamer, an antibody including heavy-chain antibody, and fragments thereof such as Fab, Fab′, and VHH, a ScFv, a spiegelmer, a peptide aptamer, vitamins and drugs such as CB1 and/or CB2 ligands.

In some other embodiments, Y, when present, is a spacer of formula (II) wherein q is 1, m is 0 or 1, p is 0 or 1, Y₁ and Y₂ are as defined above in formula (II), and Y₃ is selected from the group consisting of linear or branched C₂-C₂₀ alkyl chains, polyethylene glycol, polypropylene glycol, pHPMA, PLGA, polymers of alkyl diamines and combinations thereof, wherein the polymer preferably comprises from 2 to 40 monomers and M comprises, or consists of, a click-chemistry group, a cell-type specific ligand derived from a protein selected from transferrin, Epidermal Growth Factor (EGF), and basic Fibroblast Growth Factor βFGF, a mono- or a polysaccharide comprising one or several galactose, mannose, N-acetylgalactosamine residues, bridge GalNac, or mannose-6-phosphate, sialic acid and derivatives thereof (e.g. Neu5Ac, Neu5Acα2-6Gal, Neu5Acα2-8Neu5Ac), a MTP selected from SEQ ID NO:1 to SEQ ID NO:7, and vitamins such as folic acid.

In some other embodiments, Y, when present, is a spacer of formula (II) wherein q is 1, m is 0 or 1, p is 0 or 1, Y₁ and Y₂ are as defined above in formula (II), and Y₃ is selected from the group consisting of saturated or unsaturated, linear or branched C₂-C₄₀ hydrocarbon chains, optionally substituted, polyethylene glycol, polypropylene glycol, pHPMA, PLGA, polymers of alkyl diamines and combinations thereof and M comprises, or consists of, a click-chemistry group e.g. comprising an azido or an alkyne.

In some embodiments, Y, when present, is a spacer of formula (II) wherein q is 1, m is 0 or 1, p is 0 or 1, Y₁ and Y₂ are as defined above in formula (II), and Y₃ is selected from the group consisting of linear or branched C₂-C₂₀ alkyl chains, polyethylene glycol, polypropylene glycol, pHPMA, PLGA, polymers of alkyl diamines and combinations thereof, wherein the polymer comprises from 2 to 40 monomers and M comprises, or consists of, a click-chemistry group e.g. comprising an azido or an alkyne.

For illustration only, the AAV of the invention may comprise at least one chemically-modified cysteine in the capsid, of formula (Ia-1), (lb-1), or (Ic-1):

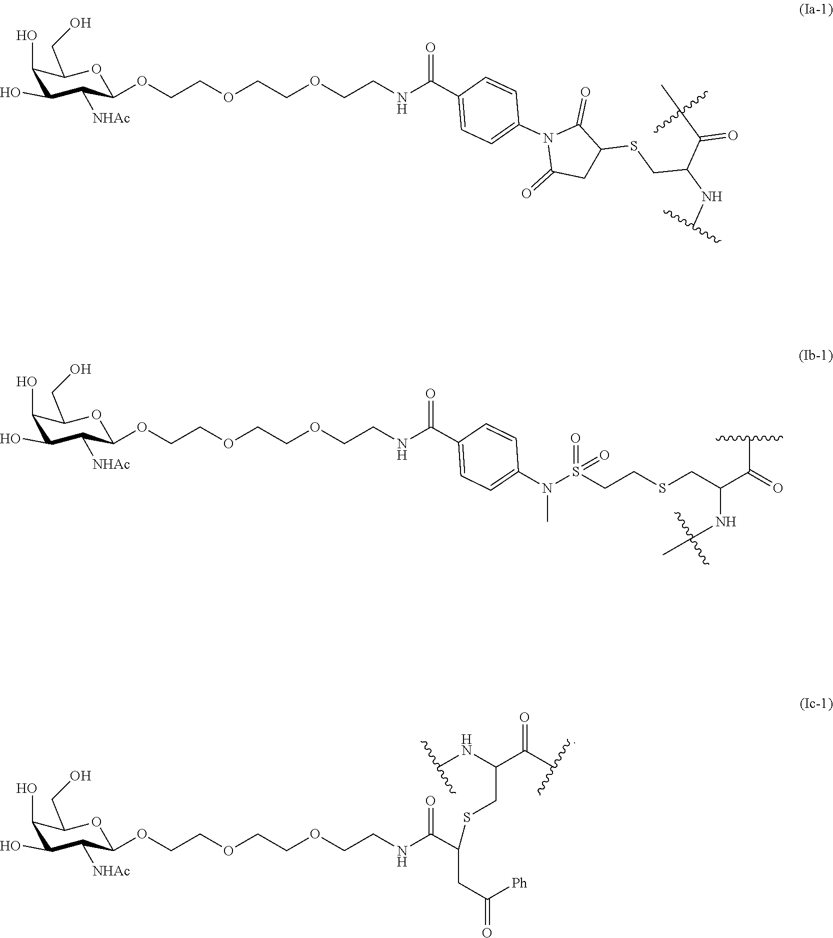

Preferably, the AAV of the invention comprises at least one chemically-modified cysteine in the capsid, of formula (Ic-1):

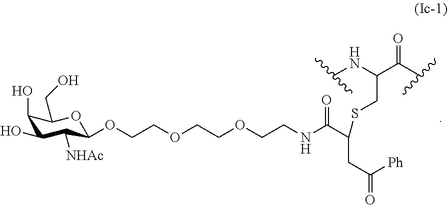

In all the embodiments described above, especially wherein the at least one chemically-modified cysteine is of formula (I), (I-II), (Ia), (Ib), (Ic), (I-IIc), (Ia-1), (lb-1), or (Ic-1), the AAV is preferably a recombinant AVV, more preferably a recombinant AAV vector.

As mentioned above, the AAV may have a “naturally-occurring” capsid or a genetically modified capsid, namely comprising one or several mutations in at least one capsid protein, namely VP1, VP2 and/or VP3. Preferably, said mutations do not introduce any additional cysteine residues, in particular any tetracysteine moiety in VP1, VP2 and/or VP3.

In some additional or alternate embodiments, the AAV may be of a serotype selected from AAV1, AAV4, AAV6, AAV7, AAV-2, AAV-3b, AAV-5, AAV-8, AAV-9 and AAVrh10, preferably AAV-2, AAV-3b, AAV-5, AAV-8, AAV-9 and AAVrh10 and more preferably AAV-2 or AAV-9.

Alternatively, the AAV is of a synthetic serotype.

In some further embodiments, the AAV of the invention may have at least one additional chemically modified amino acid residue in the capsid, which is different from a cysteine residue, e.g. a tyrosine, an arginine or a lysine residue. In some embodiments, said amino acid residue bears a modified amino group of formula (V) in its side chain:

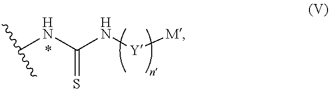

wherein:
   N* being the nitrogen of the amino group of the side chain of an amino acid residue, e.g of a lysine residue or arginine residue, and
   Y′ has the same definition as Y, n′ is 0 or 1, and M′ has the same definition as M.

In particular, Y′ may be of the following formula: —(Y_{1′})_{m′}—(Y_{2′})_{p′}—(Y_{3′})_{q′}— wherein Y_{1′}, Y_{2′}, Y_{3′}, m′, p′, and q′ have the same definition as Y₁, Y₂, Y₃, m, p, and q respectively.

It is understood that Y′, n′, Y_{1′}, Y_{2′}, Y_{3′}, m′, p′, and q′ and M′ can be the same or different as those present in the at least one chemically-modified cysteine as described above.

Said modification on the amino group can be introduced as described in WO2017212019, the content of which being incorporated herein by reference.

In some embodiments, said amino acid residue bears a modified tyrosyl group of formula (VI) in its side chain:

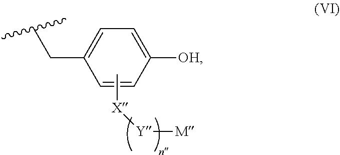

wherein
   X″ is —N═N— or

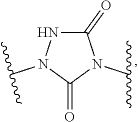 [SMILES: *n1[nH]c(=O)n(*)c1=O]

Y″ has the same definition as Y, n″ is 0 or 1, and M″ has the same definition as M.

In particular, Y″ may be of the following formula: —(Y_{1′})_{m″}—(Y_{2″})_{p″}—(Y_{3″})_{q″}— wherein Y_{1″}, Y_{2″}, Y_{3″}, m″, p″, and q″ have the same definition as Y₁, Y₂, Y₃, m, p, and q respectively.

It is understood that Y″, n″, Y_{1″}, Y_{2″}, Y_{3″}, m″, p″, and q″ and M″ can be the same or different as those present in the at least one chemically-modified cysteine as described above.

In a preferred embodiment, Y″ is of formula —(Y_{1′})_{m″}—(Y_{2″})_{p″}—(Y_{3″})_{q″}— wherein m″=1 and Y_{1″} is selected from arylene and heteroarylene comprising from 5 to 14 ring atoms, e.g. from 6 to 10 ring atoms, said arylene or heteroarylene being optionally substituted. For instance, said arylene or heteroarylene group may comprise 1, 2 or 3 substituents independently selected from halogens, —OH, NH₂, NO₂, C₁-C₃ alkyl, C₁-C₃ alkoxy, C₁-C₃ hydroxyalkyl, and C₁-C₃ haloalkyl. Said modification on the tyrosyl group can be introduced as described in WO2021005210, the content of which being incorporated herein by reference.

In a particular embodiment, the AAV of the invention has at least one additional chemically modified arginine or lysine residue in the capsid, which bears a modified amino group of formula (V) as defined above and/or at least one additional chemically modified tyrosine residue in the capsid, which bears a modified tyrosyl group of formula (VI) as defined above.

The chemical modification(s) of the capsid of the AAV may modify one or several biological functionalities and/or properties. Depending on the nature of “M” which is covalently bound on the surface of the chemically-modified AAV, said chemically-modified AAV may have one or several modified biological properties as compared to the same but non-chemically modified AAV, such as:
A modified tropism e.g. an increased selectivity of the AAV towards a specific organ, tissue or cell (either administered in vivo or transducing tissues or cells in culture) or a shifted selectivity of the AAV from one tissue/organ/cell to another, and/or
An altered immunoreactivity of the AAV, e.g. a decreased immunogenicity of the AAV and/or a decreased affinity for neutralizing antibodies, and/or said AAV triggers an altered humoral response when administered in vivo, e.g. do not generate AAV-directed neutralizing antibodies
An increased infectivity efficiency of the AAV particles, and/or
An increased transduction efficacy of the AAV towards a specific cell, tissue or organ.
A reduced cellular toxicity when transducing cells in culture
Induce cellular targeted mortality into cancer cells
Visualization/monitoring of the AAV particle upon in vivo administration or upon modification of cells in vitro with these AAV particles
Theragnostic applications; e.g. combining a therapeutic agent and a diagnostic agent

In some embodiments, the chemically-modified AAV of the invention may have a higher transduction efficiency, which may result from increased intracellular trafficking to the nuclei, a decrease in proteasome-degradation, more efficient intranuclear de-capsidation, more rapid vector genome stabilization and/or from a decrease in interaction with neutralizing antibodies and/or from a reduction of antibody-mediated clearance of AAV in vivo, as compared to the non-chemically modified AAV. In some other embodiments, the AAV may have a higher infectivity efficiency and/or an increase in selectivity for a given cell, tissue or organ as compared to the non-chemically modified AAV either in vivo or in vitro.

In some other embodiments, when the AAV is used as a drug, e.g. as a gene vector, such modified properties may result in an improvement in the therapeutic index of the AAV, which may result from decrease in the dose to administer to the patient to achieve the sought therapeutic effect and/or a decrease in the toxicity of the AAV.

In a particular embodiment, the chemically-modified AAV of the invention shows a preferential tropism for an organ or cell selected from liver, heart, brain, joints, retina and skeletal muscle.

In another or additional embodiment, the chemically-modified AAV of the invention shows a preferential tropism for cultured cells selected from, but not limited to, hepatocytes, cardiomyocytes, myocytes, neurons, motor neurons, retinal pigmented cells, photoreceptors, chondrocytes, hematopoietic stem cells (HSC) or induced pluripotent stem cells (iPS).

### Methods for Preparing the Chemically-Modified AAV of the Invention

The invention also relates to a method for chemically-modifying the capsid of an AAV, more precisely for chemically modifying at least one cysteine residue in the capsid of an AAV, which comprises incubating said AAV with a chemical reagent bearing a reactive group selected from a maleimide, a vinyl sulfonamide and a 3-(carboxy derivative)acrylamide conditions conducive for reacting said reactive group with a cysteine residue present in the capsid of the AAV so as to form a covalent bound.

As used herein a “3-(carboxy derivative)acrylamide” refers to an acrylamide substituted by a carboxy derivative in position 3. A carboxy derivative is typically a group of formula —C(O)—R₃ where R₃ is a hydrogen atom, an alkyl group, an aryl group, or a heteroaryl, said group being optionally substituted.

In a particular embodiment, the method of the invention is for obtaining a chemically-modified AAV comprising at least one chemically-modified cysteine residue in its capsid, wherein said chemically-modified cysteine residue is of formula (I):

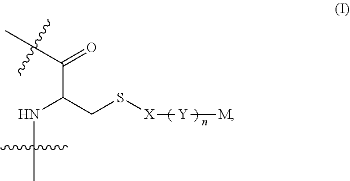

which comprises incubating the AAV with a chemical reagent selected from:

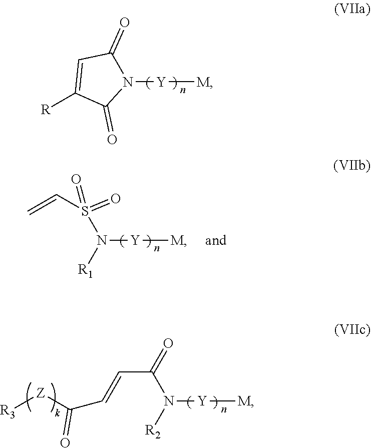

in conditions conducive for reacting said chemical reagent with a cysteine residue present in the capsid of the AAV so as to form a covalent bound.

It is understood that R, R₁, R₂, R₃, Z and k are as defined herein for formulae (a), (b), (c) and that, X, Y, n and M are as defined herein for formula (I), and that, when X is of formula (a), the chemical reagent is of formula (VIIa), when X is of formula (b), the chemical reagent is of formula (VIIb), and when X is of formula (c), the chemical reagent is of formula (VIIc).

In a particular embodiment, Y in formula (I) and in formula (VIIa), (VIIb) or (VIIc) is of formula (II) as defined herein.

Typically, the AAV particles are incubated with the chemical reagent in conditions suitable to promote the formation of a covalent bond between the thiol of the cysteine residue and said chemical reagent without impairing the structural integrity of said AAV.

The incubation may be performed in an aqueous buffer having a pH from 4 to 12, preferably a pH from 5 to 11, for instance a pH from 6 to 10 or from 6 to 9. The pH of the aqueous buffer can be from 5 to 6, from 6 to 8 or from 8 to 10.

The Inventors showed that basic pH increases the coupling rate, in particular when an acrylamide reagent (i.e. that of formula (VIIc)) is used. When a high rate of chemical coupling of cysteine residues is sought, it may be advantageous to perform the coupling at a pH from 8.0 to 11 e.g. from 8.5 to 10.5. When low rate of chemical coupling is sought, the coupling can be performed at neutral pH typically from 6.5 to 8.5 or from 7 to 8.

For maleimide and sulfonamide reagents of formula (VIIa) and (VIIb) respectively, the incubation is preferably performed at basic pH, typically at a pH from 8.0 to 10.0 such as around pH 9.5

The buffer may be selected from any appropriate buffers for biological applications such as TRIS buffer, Tris-Buffered Saline (TBS), sodium carbonate—sodium bicarbonate buffer, Good's buffers (such as AMPSO, HEPES, borate buffer, phosphate buffer e.g. PBS or Dulbecco's phosphate-buffered saline (dPBS), preferably dPBS or TBS.

The incubation may last from several minutes to several hours, for instance from 1 min to 6 h, e.g. from 3 to 5 hours. Typically, the incubation is ended when a sufficient yield of coupling is achieved.

The temperature of incubation is typically from 10° C. to 50° C. Preferably the incubation is performed at room temperature, i.e. at a temperature from 18° C. to 30° C., e.g. at around 20° C.

The incubation may be performed under stirring.

The molar ratio of the chemical reagent to the AAV particles may be from 1.10 to 1.10⁸, for instance from 1.10⁵ to 1.10⁷.

In some embodiments, the method of the invention does not contain any pre-step wherein the AAV is incubated with a reducing agent such as dithiothreitol (DTT), so as to reduce potential cysteine disulfides present in the AAV.

In some embodiments, the method of the invention comprises incubating the AAV with a chemical reagent of formula (VIIa):

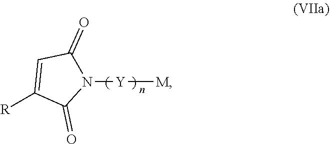

so as to obtain at least one chemically-modified cysteine residue in the capsid of formula (Ia):

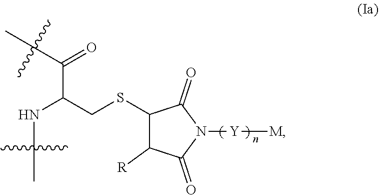

wherein Y is a spacer, n is 0 or 1, M is a functional moiety, and R is as defined herein for formula (a).

In some embodiments, the method of the invention comprises incubating the AAV with a chemical reagent of formula (VIIb):

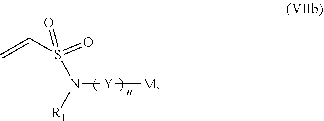

so as to obtain at least one chemically-modified cysteine residue in the capsid of formula (Ib):

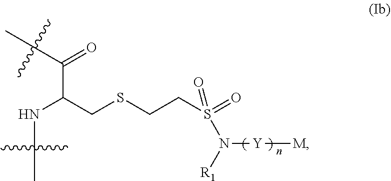

wherein:
   Y is a spacer, n is 0 or 1, M is a functional moiety, and
   R₁ is as defined herein for formula (b).

In some embodiments, the method of the invention comprises incubating the AAV with a chemical reagent of formula (VIIc):

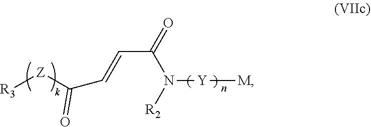

so as to obtain at least one chemically-modified cysteine residue in the capsid of formula (Ic):

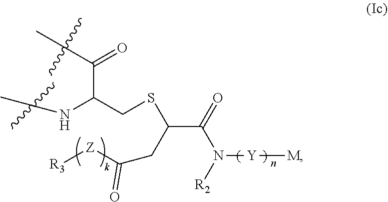

wherein:
   Y is a spacer, n is 0 or 1, M is a functional moiety, and
   R₂, R₃, Z and k are as defined herein for formula (c).

In a particular embodiment, Y in formula (I) and in formula (VIIa), (VIIb) or (VIIc) is of formula (II) as defined herein.

In a particular embodiment, the AAV prepared by the method of the invention comprises at least one chemically-modified cysteine of formula (I-IIc) as defined herein. In such embodiment, the chemical reagent is of formula (VII-IIc):

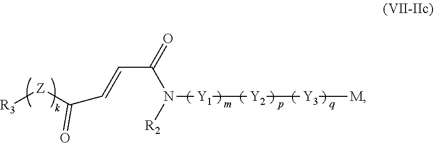

wherein R₂, R₃, Z, k, Y₁, Y₂, Y₃, m, p, q and M are as defined herein. The method of the invention may comprise one or several additional steps prior to, or after the step of incubation as described above.

For instance, the method of the invention may comprise a step of providing or producing the AAV particles to be chemically modified.

The invention may also comprise a step of providing or preparing the chemical reagent.

The chemical reagent can be produced by synthetic routes. For instance, a chemical reagent of formula (VIIc) may be prepared from an azido derivative, which is reduced into a NH₂, e.g. by hydrogenation with Pd/C as catalyst, and then forming the 3-(carboxy derivative)acrylamide e.g. by coupling the NH₂ group with a 3-(carboxy derivative)acrylate ester. As illustration only, one can refer to the synthesis of compound 7, described in the Example section.

The method of the invention may also comprise one or several additional steps following the step of incubation, such as:
a step of quenching the unreacted chemical reagent at the end of the incubation step and/or
a step of removing the unreacted reagent, e.g. by dialysis or tangential flow filtration and/or
a step of collecting the chemically modified AAV particles and/or
a step of purifying the chemically modified AAV particles and/or
a step of recovering the chemically modified AAV particles and/or
a step of formulating and/or packaging the chemically-modified AAV.

The method of the invention may further comprise a step of chemically modifying an amino acid residue other than cysteine residue of the capsid of the AAV.

For instance, said additional chemically-modified amino acid residue may bear an amino group (e.g. lysine, arginine) or a tyrosyl group (e.g. tyrosine) in its side chain.

In a particular embodiment, the method of the invention may comprise a step of incubating the AAV with a chemical reagent of formula (VIII):

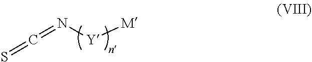

in conditions conducive to promote for reacting said chemical reagent with the amino group of an amino acid residue, e.g. a lysine residue or an arginine residue, present in the capsid of the AAV so as to form a covalent bound. Such a step enables to chemically modify an amino group present in an amino acid residue of the capsid according to the following formula:

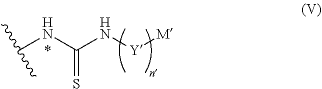

wherein:
   N* being the nitrogen of the amino group of an amino acid residue, e.g. of a lysine residue or arginine residue,
   Y′ has the same definition as Y, n′ is 0 or 1, and M′ has the same definition as M; In particular, Y′ may be of the following formula: —(Y_{1′})_{m′}—(Y_{2′})_{p′}—(Y_{3′})_{q′}— wherein Y_{1′}, Y_{2′}, Y_{3′}, m′, p′, and q′ have the same definition as Y₁, Y₂, Y₃, m, p, and q respectively.

It is understood that Y′, n′, Y_{1′}, Y_{2′}, Y_{3′}, m′, p′, and q′ and M′ can be the same or different as those present in the at least one chemically-modified cysteine as described above.

Typically such a step may be performed in an aqueous buffer, such as TRIS buffer, at a pH from 8 to 10, e.g at a pH of about 9.3 and a temperature from 10° C. to 50° C., e.g. at room temperature. More details concerning the implementation of such a step can be found in WO2017/212019, the content of which being incorporated herein by reference.

This step can be performed prior, concomitantly or after the step of chemically-modifying at least one cysteine residue in the capsid of the AAV, as described above.

In another particular embodiment, the method of the invention may comprise a step of incubating the AAV with a chemical reagent of formula (IX) or (X):

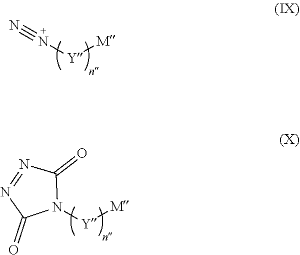

in conditions conducive to promote for reacting said chemical reagent with the amino group of an amino acid residue, e.g. a lysine residue or an arginine residue, present in the capsid of the AAV so as to form a covalent bound. Such a step enables to chemically modify a tyrosyl group present in an amino acid residue (e.g. tyrosine) of the capsid according to the following formula:

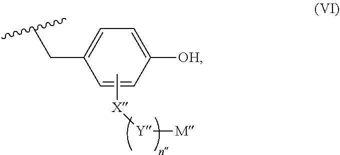

wherein
   X″ is —N═N— or

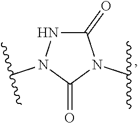 [SMILES: *n1[nH]c(=O)n(*)c1=O]

Y″ has the same definition as Y, n″ is 0 or 1, and M″ has the same definition as M. In particular, Y″ may be of the following formula: —(Y_{1′})_{m″}—(Y_{2″})_{p″}—(Y_{3″})_{q″}— wherein Y_{1″}, Y_{2″}, Y_{3″}, m″, p″, and q″ have the same definition as Y₁, Y₂, Y₃, m, p, and q respectively.

It is understood that Y″, n″, Y_{1″}, Y_{2″}, Y_{3″}, m″, p″, and q″ and M″ can be the same or different as those present in the at least one chemically-modified cysteine as described above.

It is also understood that the chemical reagent of formula (IX) enables to obtain a modified residue of formula (VI) wherein X″ is —N═N, while the chemical reagent of formula (X) enables
to obtain a modified residue of formula (VI) wherein X″ is

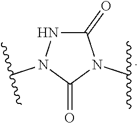 [SMILES: *n1[nH]c(=O)n(*)c1=O]

In a preferred embodiment, Y″ is of formula —(Y_{1′})_{m″}—(Y_{2″})_{p″}—(Y_{3″})_{q″}— wherein m″=1 and Y_{1″} is selected from arylene and heteroarylene comprising from 5 to 14 ring atoms, e.g. from 6 to 10 ring atoms, said arylene or heteroarylene being optionally substituted. For instance, said arylene or heteroarylene group may comprise 1, 2 or 3 substituents independently selected from halogens, —OH, NH₂, NO₂, C₁-C₃ alkyl, C₁-C₃ alkoxy, C₁-C₃ hydroxyalkyl, and C₁-C₃ haloalkyl.

The counter-anion present in the aryl diazonium salt reagent of formula (IX) can be of any type preferably TsO⁻BF₄⁻, Cl⁻, AcO⁻, PF₆⁻, TfO⁻or CF₃CO₂⁻.

Typically, such a step may be performed in an aqueous buffer, such as TRIS buffer, at a pH from 7 to 10, and a temperature from 10° C. to 50° C., e.g. at room temperature. More details concerning the implementation of such a step can be found in WO2021005210, the content of which being incorporated herein by reference.

This step can be performed prior, concomitantly or after:
the step of chemically-modifying at least one cysteine residue in the capsid of the AAV, as described above, and/or
the step of chemically-modifying at least one amino group (e.g. from arginine or lysine) in the capsid of the AAV, as described above.

Where M is a click-chemistry group, the chemically-modified AAV can undergo a supplementary step aiming at coupling a functional group Z′ by reaction with M group.

Accordingly, the invention also relates to a method for grafting a functional moiety Z′ on a cysteine residue in AAV capsid, said method comprising a step of:
preparing a chemically-modified AAV having at least one chemically modified cysteine with a functional M group, said functional M group being a click-chemistry group, preferably by the method as described above, and
coupling a functional group Z′ by click reaction with M group.

Typically, the chemically-modified AAV of the invention can be reacted with a compound of the following formula (XI):

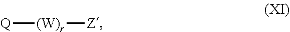

wherein:
   Q is a click-chemistry group that is able to react with M through a click chemistry reaction,
   r is 0 or 1,
   W is a spacer, and
   Z′ is a functional group different from a click-chemistry group.

Q is typically chosen among the same click-chemistry groups as M defined above, namely Q may comprise, or consist of, an azido (—N₃), an alkene, an alkyne (in particular a strained alkyne, such as cyclooctyne (OCT), aryl-less cyclooctyne (ALO), monofluorocyclooctyne (MOFO), difluorocyclooctyne (DIFO), dibenzocyclooctyne (DIBO), dimethoxyazacyclooctyne (DIMAC), biarylazacyclooctynone (BARAC), bicyclononyne (BCN), tetramethylthiepinium (TMTI, TMTH), difluorobenzocyclooctyne (DIFBO), oxa-dibenzocyclooctyne (ODIBO), carboxymethylmonobenzocyclooctyne (COMBO), or benzocyclononyne), phosphine, aldehyde, ketone, hydrazide, oxyamine, nitrile oxide, oxime, hydroxymoyl chloride, chlororoxime, nitrile imine, hydrazone, hydrazonoyl chloride, chlorohydrazone, tetrazine, isonitrile, aryl halide, aryl boronate, oligo-histidine, nickel-complex or nickel ligand.

Examples of complementary click-chemistry groups and click chemistry reactions include, but are not limited to: azido-alkyne click-chemistry (M=azide and Q=alkyne (e.g. strained intracyclic alkyne)), Staudinger Ligation (M=azide and Q=phosphine), carbonyl condensation (M=aldehyde or ketone and Q=hydrazide or oxyamine), sydnone-alkyne cycloaddition (M=sydnone and Q=alkyne), tetrazole-ene reaction (M=tetrazole and Q=alkene), nitrile-oxide-ene click chemistry (M=nitrile oxide or aldehyde, oxime, or hydroxymoyl chloride or chlororoxime and Q=alkene or alkyne), nitrile imine-ene click chemistry (M=nitrile imine or aldehyde, hydrazone, hydrazonoyl chloride or chlorohydrazone and Q=alkene or alkyne), inverse electron demand Diels-Aider ligation (M=alkene and Q=tetrazine), isonitrile-tetrazine click chemistry (M=isonitrile and Q=tetrazine), Suzuki-Miyaura coupling (M=aryl halide and Q=aryl boronate), His-tag (M=oligo-histidine and Q=nickel-complex or nickel ligand).

It is understood that, in the above-mentioned listing of click-chemistry groups involved in the click chemistry reactions, M and Q can be permuted. All the above-mentioned chemical reactions result in a covalent link.

In a particular embodiment, M is an azido (—N₃) and Q is an alkyne (such as a —C≡CH group or a strained alkyne such as those mentioned above), or Q is an azido (—N₃) and M is an alkyne (such as a —C≡CH group or a strained alkyne such as those mentioned above).

Preferably, M and Q are not thiol group (—SH), and the click chemistry reaction is not thiol-ene or thiol-maleimide reaction.

W is spacer that has typically the same definition as Y in formula (I).

Z′ is a functional group that has typically the same definition as M in formula (I) as defined herein, except that it is not a click-chemistry group. For instance, Z′ can be a targeting agent such as a cell-type targeting ligand, preferably selected from a mono- or a polysaccharide, a hormone, including a steroid hormone, a peptide such as RGD peptide, muscle targeting peptides (MTP), or Angiopep-2, a protein or a fragment thereof, a membrane receptor or a fragment thereof, an aptamer, an antibody including heavy-chain antibody, and fragments thereof such as Fab, Fab′, and VHH, a ScFv, a spiegelmer, a peptide aptamer, an oligonucleotide, a vitamin and small chemical molecules such as drugs e.g. CB1 and/or CB2 ligands.

For instance, Z′ can be a cell-type specific ligand derived from proteins such as transferrin, Epidermal Growth Factor (EGF), and basic Fibroblast Growth Factor βFGF, muscle targeting peptides as described above and from mono- or polysaccharides, e.g. comprising one or several galactose, mannose, mannose-6-phosphate, N-acetylgalactosamine, or bridge GalNac, sialic acid and derivatives thereof (e.g. Neu5Ac, Neu5Acα2-6Gal, Neu5Acα2-8Neu5Ac), CB1 and/or CB2 ligands and vitamins such as folic acid.

The conditions implemented for the click chemistry reaction are well-known to the skilled artisan. The click reaction may be “bioorthogonal” or “biocompatible”, this means that the reagents involved in the click reaction may react selectively and rapidly with each other in the presence of a plurality of biological entities. In some embodiments, the click reaction may be conducted in media comprising living cells, without interfering with cellular process.

For instance, biocompatible or biorthogonal click reactions encompass metal-free click-reactions (i.e. which do not require metal catalysts). Examples of metal-free click reactions are depicted hereunder:

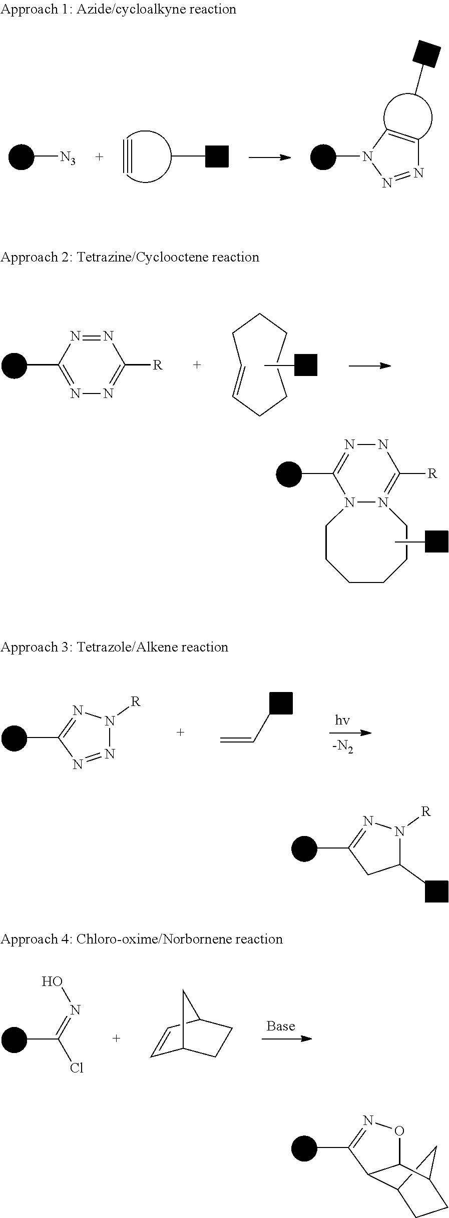

Other metal-free click-reactions of interest are for instance iminosydnone or sydnone derivatives-strained alkyne cycloadditions as described in PCT/EP2015/060805 and PCT/EP2015/063750, the disclosure of which being incorporated herein by reference.

For a review concerning biorthogonal chemistry, including click-chemistry, one can refer to Sletten and Bertozzi, Angew. Chem. Int. Ed. Engl. 2009, 48(38):6974-6998, the disclosure of which being incorporated herein by reference.

Preferred click-reactions are free-metal reactions, i.e. click-reactions which do not require the presence of a metal catalyzer such as copper salt.

In a particular embodiment, the click reaction of interest is a strain promoted alkyne-azide cycloaddition (SPAAC), which means that M can be an azido group and Q can be a strained alkyne as described above, or vice versa.

The invention also relates to the chemically-modified AAV obtainable, or obtained, by the method of the invention as described above.

In a further aspect, the invention also relates to a method for modifying one or several biological properties of the AAV, more precisely that of a recombinant AAV intended to be used as gene vector in gene therapy. Indeed, depending on the nature of “M” moiety, the method for chemically-modifying the capsid of an AAV, more precisely for chemically modifying at least one cysteine residue in the capsid of an AAV, as described above may enable to:
Modify the tropism e.g. increase selectivity of the AAV towards a specific organ, tissue or cell (either administered in vivo or transducing tissues or cells in culture) or shift the selectivity of the AAV from one tissue/organ/cell to another, and/or
Alter immunoreactivity of the AAV, e.g. a decrease immunogenicity of the AAV and/or decrease affinity for neutralizing antibodies, and/or said AAV triggers an altered humoral response when administered in vivo, e.g. do not generate AAV-directed neutralizing antibodies, and/or
Increase infectivity efficiency of the AAV particles, and/or
Reduce off-target effects i.e. transduce cells that are not necessary for providing a benefit of the drug and could be even detrimental.
Increase transduction efficacy of the AAV towards a specific cell, tissue or organ.
Reduce cellular toxicity when transducing cells in culture
Induce cellular targeted mortality into cancer cells
Enabling visualization/monitoring of the AAV particle upon in vivo administration or upon modification of cells in vitro with these AAV particles
combine a therapeutic agent and a diagnostic agent in the AAV

### Uses of the AAV of the Invention

The chemically-modified AAV of the invention can be used as a research tool or as a medicament, for instance as vectors for the delivery of therapeutic nucleic acids such as DNA or RNA and or as a diagnostic mean e.g. as an imaging agent or combination of both, including theragnostic use.

In some embodiments, the chemically-modified AAV of the invention is used for delivering a nucleic acid into a cell, in particular an exogenous nucleic acid such as a transgene, and is thus a recombinant AAV.

The recombinant AAV can be administered to the cell in vivo, ex vivo or in vitro. The cell may be derived from any mammal including humans, primates, cows, mice, sheeps, goats, pigs, rats, and the like. The cell may be of any type, including hepatocytes, cardiomyocytes, myocytes, neurons, motor neurons, retinal pigmented cells, photoreceptors, chondrocytes, hematopoietic stem cells (HSC) or induced pluripotent stem cells (iPS).

The recombinant AAV of the invention may be used to deliver a therapeutic nucleic acid of interest in a subject. The invention thus relates to a method for delivering a therapeutic nucleic acid of interest in a subject in need thereof comprising administering the chemically-modified AAV of the invention to a subject in need thereof. The recombinant AAV of the invention can be delivered by any appropriate route to the subject. Appropriate administration routes encompass, without being limited to, inhalational, topical, intra-tissue (e.g. intramuscular, intracardiac, intrahepatic, intrarenal), conjunctical (e.g. intraretinal, subretinal), mucosal (e.g. buccal, nasal), intra-articular, intravitreal, intracranial, intravascular (e.g. intravenous), intraventricular, intracisternal, intraperitoneal and intralymphatic routes. Typically, the route of administration is selected depending on the targeted tissue/organ, namely depending on the tissue/organ in which the transduction is sought.

The dose of AAV to administer to the subject is typically determined by the skilled artisan in view of the specific features of the subject, the therapeutic effect sought and the targeted tissue/organ. One single administration or several administrations of the AAV may be requested to achieve the sought therapeutic effect. The AAV of the invention is typically administered in the form of a pharmaceutical composition, namely as a mixture with one or several pharmaceutical excipients.

The conditions to be treated by the administration of the AAV may be of any type, and includes genetic disorders as well as acquired disorders. Genetic disorders of interest encompass genetic muscle disorders such as Duchenne Muscular Dystrophy, leukodystrophy, spinal muscular atrophy (SMA), hemophilia, sickle disease, and inherited retinal dystrophy. The chemically-modified AAV may also be used for treating disorders such as cancers, arthritis, arthrosis, congenital and acquired cardiac diseases, Parkinson disease, Alzheimer's disease as well as infectious diseases such as hepatitis C.

Another object of the invention is a pharmaceutical composition comprising a chemically-AAV of the invention and at least one pharmaceutically acceptable excipient. The pharmaceutical excipients may be selected from well-known excipients such as carriers, preservatives, antioxidants, surfactants, buffer, stabilizer agents, and the like.

The invention further relates to an in vivo or ex vivo method for delivering a nucleic acid of interest in a cell comprising contacting the chemically-modified AAV of the invention with the cell. The cell may be from the patient. After the transduction, the cell may be transplanted to the patient in need thereof. The cell may be, for instance, hematopoietic stem cells. The nucleic acid of interest may be of any type and is selected depending on the sought effect.

For instance, the AAV may comprise an exogenous gene expression cassette. Said cassette may comprise a promoter, the gene of interest and a terminator. As another example, the AAV of the invention may comprise a DNA template for homologous recombination in cells. Such a recombinant AAV can be used in combination with gene editing tools, for promoting homologous recombination in targeted cells. The gene editing tools can be of any type, and encompass, without being limited to, CRISPR/Cas9, Zinc Finger Nuclease, meganuclease as well as RNA and DNA encoding said proteins.

The invention also relates to a host cell transfected with a chemically modified AAV of the invention, said host cell can be of any type.

For instance, said host cell may be hepatocytes, cardiomyocytes, myocytes, neurons, motor neurons, retinal pigmented cells, photoreceptors, chondrocytes, hematopoietic stem cells (HSC) or induced pluripotent stem cells (iPS).

Further aspects and advantages of the present invention are disclosed in the following experimental section, which should be regarded as illustrative and not limiting the scope of the present application.

### EXAMPLES

### Example 1: Synthesis of a Ligand According to the Invention

### 1. Synthesis of a Ligand According to the Invention

The GalNAC-acrylamide ligand (L) of the following formula:

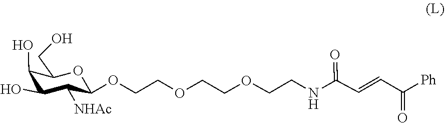

was prepared from 3-benzoylacrylic acid according to the following reaction steps:

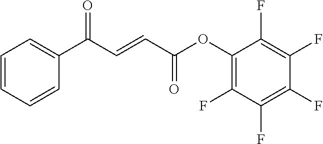 [SMILES: O=C(/C=C/C(=O)c1ccccc1)Oc1c(F)c(F)c(F)c(F)c1F]

3-benzoylacrylic acid (500 mg, 2.8 mmol) was dissolved in anhydrous DCM and a mixture of pentafluorophenyl trifluoroacetate (530 μL, 3.1 mmol) and DIPEA (990 μL, 5.7 mmol) was added with stirring. The reaction mixture was stirred for 1 h 30 at RT. Solvents were removed under reduced pressure and the crude product purified by silica-gel column chromatography (50% DCM/petroleum ether) to give compound A (793 mg, 2.31 mmol) as a yellow solid. Yield=66.4%.

¹H NMR (300 MHz, CDCl₃, 298.15 K): δ_{H} 8.17 (d, 1H, 3J=15.5 Hz, H³), 8.04 (m, 2H, H^{arom}), 7.67 (m, 1H, H^{arom}), 7.56 (m, 2H, H^{arom}), 7.12 (d, 1H, ³J=15.5 Hz, H²)

¹³C NMR (100.62 MHz, CDCl₃, 298.15 K): δ_{C} 188.54 (1C), 161.58 (1C), 140.34 (1C), 136.24 (1C), 134.53 (1C), 129.24 (2C), 129.12 (2C), 128.71 (1C)

HRMS (ESI⁺): m/z calculated for C₁₆H₉F₅O₃ [M+H]⁺ 343.0389 found 343.0394

### Preparation of Compound 1

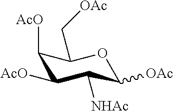

N-acetylgalactosamine (2 g, 9.04 mmol) was dissolved in anhydride acetic and pyridine (1/1, 2.5 mL/mmol), along with 4-dimethylaminopyridine (110 mg, 0.904 mmol). The mixture was stirred for at room temperature, concentrated under vacuum, dissolved in dichloromethane (100 mL), washed with an aqueous solution of HCl (1M), a saturated solution of aqueous NaHCO₃, water and brine. The organic layer was dried over MgSO4, filtered and concentrated under vacuum to give the product as a colorless amorphous solid (3.51 g, quant. yield).

¹H NMR (400 MHz, MeOD, 298.15 K): δ_{H} 6.17 (d, 1H, ³J=3.6 Hz, H¹), 5.48 (dd, 1H, ³J=3.1 Hz and 1.3 Hz, H⁴), 5.21 (dd, 1H, ³J=11.7 Hz, and 3.2 Hz, H³), 4.57 (ddd, 1H, ³J=11.7 Hz and 9.1 Hz and 3.6 Hz, H²), 4.38 (dd, 1H, ³J=1.1 Hz and 6.7 Hz, H⁵), 4.13 (dd, 1H, ³J=11.2 Hz and 6.7 Hz, H^{6a}), 4.05 (dd, 1H³J=11.2 Hz and 6.6 Hz, H⁶b), 2.16 (s, 3H, CH₃), 2.15 (s, 3H, CH₃), 2.01 (s, 3H, CH₃), 1.97 (s, 3H, CH₃), 1.92 (s, 3H, CH₃)

HRMS (ESI⁺): m/z calculated for C₁₆H₂₃NO₁₀Na [M+Na]⁺412.1216 found 412.1220

### Preparation of Compound 2

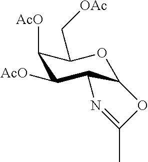

TMSOTf (0.47 mL, 2.62 mmol) was added to a stirred solution of 1 (300 mg, 0.77 mmol) in anhydrous DCM (7.7 mL) at room temperature under N₂. The reaction mixture was stirred overnight at 45° C. and then quenched by addition of NEt₃ (0.22 mL, 1.54 mmol) at 0° C. The mixture was diluted with CH2Cl2 (100 mL), washed with a saturated solution of aqueous NaHCO₃, water and brine, dried over MgSO4, filtered and concentrated under vacuum. The resulting crude oxazoline (brown oil) (231 mg, 91% yield) was used without further purification.

¹H NMR (300.13 MHz, CDCl₃, 298.15 K): δ_{H} 6.00 (d, 1H, ³J=6.7 Hz, H¹), 5.46 (t, 1H, ³J=3 Hz, H⁴), 4.92 (dd, 1H, ³J=7.4 Hz and 3.3 Hz, H³), 4.24 (dd, 1H, ³J=6.8 Hz and 2.9 Hz, H⁵), 4.20 (dd, 1H, ³J=11.1 Hz and 5.8 Hz, H^{6a}), 4.11 (dd, 1H, ³J=11.1 Hz and 6.9 Hz, H^{6b}), 4.00 (td, 1H, ³J=7.3 Hz and 1.3 Hz, H²), 2.12 (s, 3H, CH₃), 2.06 (s, 9H, 3×CH₃)

HRMS (ESI⁺): m/z calculated for C₁₄H₂₀NO₈ [M+H]⁺ 330.1188 found 330.1189

### Preparation of Compound 3

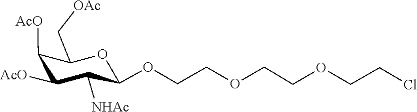

2-(2-(2-chloroethoxy)ethoxy)ethanol (1.36 mL, 9.38 mmol) was added to a stirred solution of 2 (2.81 g, 8.53 mmol) in anhydrous DCM (85 mL) at room temperature under N₂, followed by the addition of TMSOTf (0.77 mL, 4.27 mmol) at 0° C. The reaction mixture was stirred overnight at room temperature and then quenched by addition of TEA (0.75 mL, 5.55 mmol) at 0° C. The mixture was diluted in DCM (100 mL), washed with water, brine, dried over MgSO4, filtered and concentrated under vacuum to give the β-anomer as a brown liquid oil (4.23 g, quant. yield).

¹H NMR (300.13 MHz, CDCl₃, 298.15 K): δ_{H} 6.27 (d, 1H, ³J=9.5 Hz, NH), 5.29 (dd, 1H, ³J=3.4 Hz and 0.6 Hz H⁴), 4.98 (dd, 1H, J=11.2 Hz and 3.4 Hz, H³), 4.78 (d, 1H, J=8.6 Hz, H¹), 4.25 (m, 1H, H²), 4.13 (t, 2H, J=6.5 Hz, 2×H⁶), 3.92-3.58 (m, 13H, 5×CH2, H⁶ and H⁵), 2.15 (s, 3H, CH₃), 2.03 (s, 3H, CH₃), 1.97-1.98 (s, 6H, 2×CH3)

¹³C NMR (100.62 MHz, CDCl₃, 298.15 K): δ_{C} 170.77 (1C), 170.70 (1C), 170.64 (1C), 170.55 (1C), 102.53 (1C, C¹), 71.99 (1C), 71.42 (1C, C³), 71.20 (1C, C⁵), 70.76 (1C), 70.68 (1C), 70.40 (1C), 68.64 (1C), 66.79 (1C, C⁴), 61.72 (1C, C⁶), 50.62 (1C, C²), 43.06 (1C), 23.43 (1C), 20.90 (1C), 20.85 (1C), 20.80 (1C)

### Preparation of Compound 4

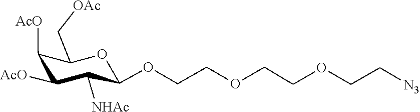

Compound 3 (2.083 g, 4.18 mmol) was dissolved in dry DMF (41.8 mL) and sodium azide (1.36 g, 20.9 mmol) was added at room temperature under N₂ atmosphere along with sodium iodide (63 mg, 0.42 mmol). The mixture was stirred for 16 h at 70° C., then concentrated under vacuum, dissolved in dichloromethane (150 mL), washed with a saturated solution of aqueous NaHCO₃, water and brine. The organic layer was dried MgSO4, filtered, concentrated under vacuum and purified with a column chromatography to give the corresponding azide as a brown liquid oil (1.27 mg, yield 60%).

¹H NMR (300.13 MHz, CDCl₃, 298.15 K): δ_{H} 6.06 (d, 1H, ³J=6.14 Hz, NH), 5.31 (dd, 1H, ³J=3.3 Hz and 0.8 Hz, H⁴), 5.07 (dd, 1H, ³J=11.2 Hz and 3.4 Hz, H³), 4.77 (d, 1H, ³J=8.6 Hz, H¹), 4.18-4.03 (m, 1H, H²), 4.08 (m, 2H, H⁶), 3.93-3.78 (m, 3H, CH₂, H⁵), 3.76-3.59 (m, 8H, 4×CH₂), 3.45 (t, 2H, ³J=4.9 Hz, H¹³), 2.08 (s, 3H, CH₃), 1.98 (s, 3H, CH₃), 1.93-1.91 (s,6H, 2×CH₃).

¹³C NMR (100.62 MHz, CDCl₃, 298.15 K): δ_{C} 170.72 (1C), 170.57 (1C), 170.55 (1C), 170.48 (1C), 102.33 (1C, C¹), 71.72 (1C), 71.01 (1C, C³), 70.85 (1C, C⁵), 70.79 (1C), 70.54 (1C), 69.93 (1C), 68.71 (1C), 66.88 (1C, C⁴), 61.71 (1C, C⁶), 50.95 (1C, C²), 50.55 (1C, C¹³), 23.33 (1C), 20.86 (1C), 20.80 (2C)

HRMS (ESI⁺): m/z calculated for C₂₀H₃₂N₄O₁₁Na [M+Na]527.1965 found 527.1984

### Preparation of Compound 5

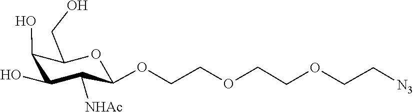

To a solution of 4 (980 mg, 1.94 mmol) in methanol, sodium methoxide (0.1 equiv.) in methanol was added and stirred at room temperature for 6 h, neutralized with Dowex ion exchange (H+) resin, filtered and concentrated in vacuo to give the corresponding azide 5.

¹H NMR (300.13 MHz, CDCl₃, 298.15 K): δ_{H} 4.44 (d, 1H, ³J=8.4 Hz, H¹), 4.00-3.86 (m, 3H, CH₂ and H²), 3.83 (dd, 1H, J=3.2 Hz and 0.4, H⁴), 3.80-3.62 (m, 10H, 4×CH2 and H⁶), 3.58 ( dd, 1H, ³J=10.7 Hz and 3.3 Hz, H³), 3.47 (td, 1H, ³J=5.3 Hz and 0.8 Hz, H⁵), 3.39 (t, 2H, ³J=5.0 Hz, H¹³), 1.99 (s, 3H, CH₃)

¹³C NMR (100.62 MHz, CDCl₃, 298.15 K): δ_{C} 174.17 (1C), 103.09 (1C, C¹), 76.75 (1C, C⁵), 73.58 (1C, C³), 71.66 (1C), 71.59 (1C), 71.55 (1C), 71.07 (1C), 69.78 (1C), 69.71 (1C, C⁴), 62.52 (1C, C⁶), 54.27 (1C, C²), 51.77 (1C, C¹³), 19.01 (1C)

HRMS (ESI⁺): m/z calculated for C₁₄H₂₆N₄₀₈Na [M+Na]⁺401.1648 found 401.1659.

### Preparation of Compound 6

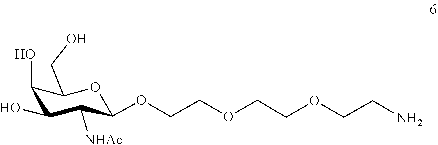

To a solution of the azide 5 (636 mg, 1.68 mmol) in methanol (17 mL) was added PTSA (289 mg, 1.68 mmol), followed by the addition of 10% of Pd-C(10% w). The resulting suspension was stirred under H₂ atmosphere for 18 h. The Pd/C was removed by filtration through Celite® and the filtrate was evaporated under reduced pressure to give the ammonium salt, as confirmed by ¹H NMR. The crude of the reaction was dissolved in a mixture 1:1 H₂O/MeOH (20 mL), followed by addition of Amberlite IRN78 basic resin. After 1 h of stirring at 20° C., the reaction mixture was filtrated and evaporated under reduce pressure to give the corresponding amine 6 as a white solid (550 mg, 93% yield) used in the next step without further purification

¹H NMR (300.13 MHz, MeOD, 298.15 K): δ_{H} 4.43 (d, 1H, ³J=8.4 Hz, H¹), 3.95 (m, 2H, CH₂), 3.92 (m, 1H, H²), 3.82 (m, 1H, H⁴), 3.79-3.45 (m, 12H, 4×CH2 and H³ and H⁵ and 2×H⁶), 2.79 (t, 2H, ³J=5.5 Hz, H¹³), 1.99 (s, 3H, CH₃)

¹³C NMR (100.62 MHz, MeOD, 298.15 K): δ_{C} 174.13 (1C, C), 103.57 (1C, C¹), 76.77 (1C, C⁵), 73.48 (1C, C³), 73.45 (1C, CH₂), 71.61 (1C, CH₂), 71.52 (1C, CH₂), 69.79 (1C, C⁶), 69.69 (1C, C⁴), 62.54 (1C, CH₂), 56.28 (1C, CH₂), 54.48 (1C, C²), 42.06 (1C, C¹³), 22.88 (1C, CH₃)

HRMS (ESI⁺): m/z calculated for C₁₄H₂₄N₂₀₈Na [M+Na]375.1743 found 375.1743

### Final Step for Obtaining the Ligand of the Invention: Compound GalNAc-Acrylamide (L)

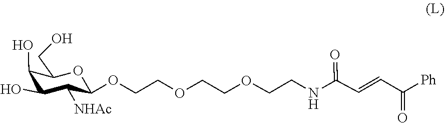

Compound 6 (100 mg, 0.28 mmol) was dissolved in anhydrous DMF and a mixture of A (86 mg, 0.25 mmol) and DIPEA (90 μL, 0.50 mmol) in anhydrous DCM was added with stirring. The reaction mixture was stirred for 1 h at RT. Solvents were removed under reduced pressure and the crude product purified by silica-gel column chromatography (DCM/MeOH 85/15) to give the GalNAc-Acrylamide (L) (47 mg, 92 mol) as an oil. Yield=33%

¹H NMR (300.13 MHz, MeOD, 298.15 K): δ_{H} 8.03 (m, 2H, Hₐᵣₒₘ), 7.88 (d, 1H, ³J=15.4 Hz, H¹⁴), 7.66 (m, 1H, Hₐᵣₒₘ), 7.66 (m, 2H, Hₐᵣₒₘ), 7.06 (d, 1H, ³J=15.4 Hz, H¹⁵), 4.44 (d, 1H, ³J=8.4 Hz, H¹), 4.00-3.90 (m, 3H, H² and CH₂), 3.84 (m, 1H, H⁴), 3.81-3.56 (m, 11H, H³ and 2×H⁶ and 4×CH₂), 3.55-3.46 (m, 3H and H⁵ and H¹²), 1.98 (s, 3H, CH₃)

¹³C NMR (100.62 MHz, MeOD, 298.15 K): δ_{C} 191.6 (1C, C¹⁶), 174.2 (1C, C¹⁷), 166.8 (1C, C¹³), 138.30 (1C, C° ^{m}), 136.6 (1C, C¹⁵), 134.9 (1C, C° ^{m}), 134.1 (1C, C¹⁴), 130.0 (1C, 2×C^{arom}), 129.8 (1C, 2×C^{arom}), 103.1 (1C, C¹), 76.75 (1C, C⁵), 73.44 (1C, C³), 71.6-71.4 (3C, 2×CH₂ and C⁶), 70.4 (1C, CH₂), 69.8 (1C, CH₂), 69.71 (1C, C⁴), 62.6 (1C, CH₂), 54.3 (1C, C²), 40.8 (1C, CH₂), 23.1 (1C, CH₃)

HRMS (ESI⁺): m/z calculated for C₂₄H₃₄N₂O₁₀Na [M+Na]⁺533, 2112 found 533, 2111

### 2. Synthesis of Compound C (Comparative)

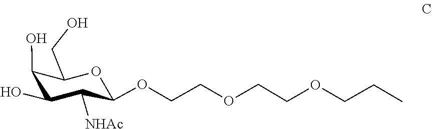

The synthesis of compound C was prepared as described in Chemical Science, 2020, 11, 1122-1131.

### Example 2: Preparation Chemically-Modified AAV

### AAV2 and AAV9 Production and Purification

AAV vectors were produced from two plasmids: (i) pHelper, PDP2-KANA encoding AAV Rep2-Cap2 and adenovirus helper genes (E2A, VA RNA, and E4) for AAV2 vectors or PDP9-KANA encoding AAV Rep2-Cap9 and adenovirus helper genes (E2A, VA RNA, and E4) for AAV9 vectors and (ii) the pVector ss-CAG-eGFP containing the ITRs. All vectors were produced by transient transfection of HEK293 cells with calcium phosphate-HeBS method. AAV2 transfected cells were harvested 48 h after transfection and treated with Triton-1% and benzonase (25U/mL) for 1 h at 37° C. AAV9 transfected cells were harvested 96 H post transfection, the supernatant is only precipitated at 5+/−3° C. over night with PEG. The precipitated supernatant is then centrifuged. The supernatant is discarded and the PEG-pellet is resuspended in TBS before benzonase digestion. Vectors were purified by double cesium chloride (CsCl) gradient ultracentrifugation. The viral suspension was then subjected to four successive rounds of dialysis under slight stirring in a Slide-a-Lyzer cassette (Pierce) against dPBS (containing Ca++ and Mg++).

### Coupling and Purification

The general proceeding is shown in FIG. 1.

AAV2-CAG-GFP or AAV9-CAG-GFP (1012 vg, 2.49 nmol, 100 μL) were added to a solution of dPBS buffer (100 μL or 900 μL) containing the GalNAc-Acrylamide Ligand (L) or comparative compound (C) at different molar ratios (3E5 or 3E6) and incubated during 4 h at RT at pH 7.4. The solutions containing the vectors were then dialyzed against dPBS+0.001% Pluronic to remove free molecules that were not bond to the AAV capsid. For the HPLC/MS analyses the solutions (200 μL) were directly lyophilized without dialyses.

### Example 3: Characterization of Chemically-Modified AAVs (Invention and Comparative)

Quantification of AAV vector genomes

3 μL of AAV were treated with 20 units of DNase I (Roche #04716728001) at 37° C. for 45 min to remove residual DNA in vector samples. After the treatment with DNase I, 20 μL of proteinase K 20 mg/mL (MACHEREY-NAGEL #740506) was then added and incubated at 70° C. for 20 min. Extraction columns (NucleoSpin® RNA Virus) were then used to extract DNA from purified AAV vectors.

Quantitative real time PCR (qPCR) was performed with a StepOnePlus™ Real-Time PCR System Upgrade (Life technologies). All PCRs were performed in a 20 μL final volume PCR including primers and probe targeting the ITR2 sequence, 2 PCR Master Mix (TaKaRa) and 5 μL of template DNA (plasmid standard, or sample DNA). qPCR was carried out with an initial denaturation step at 95° C. for 20 seconds, followed by 45 cycles of denaturation at 95° C. for 1 second and annealing/extention at 56° C. for 20 seconds. Plasmid standards were generated with seven serial dilutions (containing 108 to 102 copies of plasmid) according to [S. D'Costa et al., Molecular therapy. Methods & clinical development, 2016].

### Western Blotting and Silver Staining

All vectors were denatured at 100° C. for 5 min using Laemmli sample buffer and separated by SDS-PAGE 10% Tris-glycine polyacrylamide gels (Life Technologies). Precision plus Protein All Blue Standards (BioRad) was used as a molecular-weight size marker. Following electrophoresis, gels were either silver stained (PlusOne Silver staining Kit, protein, GE Healthcare) or transferred onto nitrocellulose membranes for Western blot analysis. After transferring the proteins to nitrocellulose membrane using a transfer buffer (25 mM tris/192 mM Glycine/0.1 (w/v) SDS/20% MeOH) for 1 h at 150 mA in a Trans-Blot SD Semi-Dry Transfer Cell (Bio-Rad), the membrane was saturated with 5% semi-skimmed milk in PBS-Tween (0.1%) or with 1% gelatin, 0.1% Igepal in PBS-Tween (0.01%) during 2 h at RT. After saturation, the membrane was probed with the corresponding antibody or lectin overnight at 4° C. Three washes were carried out between each stage to remove unbound reagents with PBS-Tween (0.1%) for 15 min at RT. Bands were visualized by chemiluminescence using alkaline phosphatase (AP) or horseradish peroxidase (HRP)-conjugated secondary antibodies and captured on X-ray film.

### Immuno Dot-Blot Analysis

AAV vectors were loaded at a dose of 1010 vg on a nitrocellulose paper soaked briefly in PBS prior to assembling the dot blot manifold (Bio-Rad). Nitrocellulose membrane containing the vectors was treated as for Western blotting.

### Enzymatic Digestion of AAV Vectors and LC-MS/MS Peptide Analysis

AAV samples (1×1013 vg/mL) were prepared with the ProteinWorks™ eXpress kit (Waters Corporation) as previously described [Blanchard et al., Journal of Lipid Research, 2020]. Samples (40 μL) were incubated for 10 min at 80° C. in digestion buffer (ammonium bicarbonate 50 mM, pH 8, 100 μL) and RapidGest detergent solution (7 mg/mL, 10 μL), reduced for 20 min at 60° C. with dithiothreitol (70 mM, 20 μL), alkylated for 30 min at room temperature in the dark with iodoacetamide (142 mM, 30 μL), and digested overnight at 37° C. (˜16 h) with trypsin (7 mg/mL in HCl 1 mM, 30 μL). Enzymatic digestion was stopped with 20% trifluoroacetic acid (TFA; 5 μL). After 15 min at 45° C., the precipitate was removed by centrifugation (15 min; 10° C.; 10,000 rpm), and supernatants were cleaned on 30 mg Oasis HLB cartridges (Waters Corporation), which were conditioned (100% methanol; 1 mL), equilibrated (100% water; 1 mL), loaded (sample; ˜200 μL), washed (5% methanol; 1 mL), and eluted (80% methanol; 500 μL). The eluates were dried under nitrogen (45° C.), reconstituted with 5% acetonitrile containing 0.1% formic acid (100 μL), and injected (10 μL) into the LC-HRMS system. LC-HRMS analyses were performed on a Synapt G2 HRMS Q-TOF mass spectrometer equipped with an ESI interface operating in the positive mode and an Acquity H-Class UPLC device (Waters Corporation). Samples were injected (10 μL) onto a Acquity CSH C18 Peptide reversed-phase column (1.7 m; 2.1×100 mm; 130 Å) held at 60° C. Digest peptides were then eluted over 20 min with a linear gradient of mobile phase B (100% acetonitrile) in mobile phase A (5% acetonitrile), each containing 0.1% formic acid, and at a flow rate of 250 μL/min. Mobile phase B was kept constant at 1% for 1 min, then linearly increased from 1% to 80% for 15 min, kept constant for 1 min, returned to the initial condition over 1 min, and kept constant for 1 min before the next injection. The full-HRMS mode was applied for peptides (scan range 100-4,000 m/z) at a mass resolution of 25,000 full-widths at half maximum. The ionization settings were as follows: capillary voltage, +3 kV; cone voltage, 30 V; desolvation gas (N2) flow rate, 1000 μL/h; desolvation gas/source temperatures, 450/120° C. Leucine enkephalin solution (2 g/mL, 50% acetonitrile) was infused at a constant flow rate of 10 μL/min in the lockspray channel, allowing for correction of the measured m/z throughout the batch (theoretical m/z 556.2771 in positive mode). Data acquisition and processing were achieved using MassLynx® software (version 4.1, Waters Corporation). MS profiles of the peaks on the chromatogram allowed to identify peptides and confirm conversion to the conjugated compounds. Tandem mass spectrometry (MS/MS) fragmentation was then performed on the major ion peak (mono, double or triple charged) to identify the location of conjugation with a collision energy ramping from 15 to 40 eV.

### Results

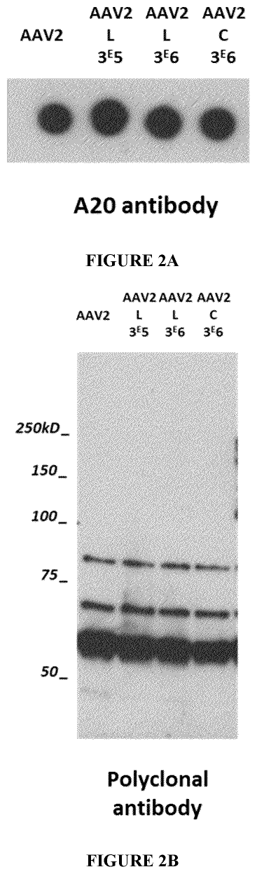

The results are shown in FIG. 2A to FIG. 3C.

The integrity of the capsids after coupling reaction with the ligand (L) of the invention was assessed by dot blot analysis using immunostaining with A20 antibody. A20 antibody recognizes the assembled AAV2 capsid. The result is shown in FIG. 2A. The positive dots with A20 antibody indicate that AAV2 remained intact after the coupling procedure with Ligand (L).

A further evidence of the integrity of the capsid is provided the western blot analyses.

Immunostaining performed with polyclonal antibodies specific to capsid proteins show that the chemical coupling did with the ligand of the invention not impair the integrity of capsid subunits of AAV2 (FIG. 2B).

The specific coupling of the ligand of the invention to cysteine residues capsids were evidenced by LC-MS/MS analysis. Some results of the LC-MS/MS are shown in FIGS. 3A-3B-3C. The “bottom-up” strategy was used to validate the specific conjugation of cysteine residues within AAV2 and AAV9. Such mass spectrometry-based protocols involve the analysis of peptide mixtures resulting from the enzymatic proteolysis of proteins. Peptides are then separated by liquid chromatography and detected by mass spectrometry according to their exact (±10 ppm) mass-to-charge ratio (m/z), taking into account the expected mass shift brought by the chemical modification. To confirm the location of the modifications, peptides are then fragmented using the MS/MS mode. Fragmentation patterns allow to specify the location of the modification within peptide sequences.

The in silico selection of proteotypic peptides led to 5 or 4 candidates carrying cysteine residues for AAV2 or AAV9, respectively. The most specific and detectable of them were selected to optimize the assay sensitivity and specificity. Two peptide candidates were particularly of interest as they were common to both AAV2 and AAV9 and were carried by VP1, VP2 and VP3: FHCHFSPR (SEQ ID NO:8) (cysteine located in position 289, the numbering referring to the amino acid position in AAV2 VP1) and SSFYCLEYFPSQMLR (SEQ ID NO:9) (cysteine located in position 394, the numbering referring to the amino acid position in AAV2 VP1). These peptides were successfully detected by mass spectrometry at their exact m/z in the control samples (unmodified AAVs) but not in the chemically-modified samples. In contrast, by taking into account the expected mass shift in these specific m/z (+510 Da), two peptides were detected in the chemically-modified samples but not in the control samples, suggesting the success of the chemical reaction i.e. the specifical chemical coupling at cysteine at positions 289 and 394.

To ensure that the modification has occurred on the cysteine residues only, MS/MS fragmentation was then performed on the double charged precursor ions. Fragmentation patterns clearly showed that the chemical modification occurred on cysteine residues since the specific mass shift was only observed on peptide fragments carrying the cysteine in comparison to the unmodified peptides.

To validate the specificity of the chemical reaction, two additional peptides that did not carry cysteine residue were also investigated by using the same strategy (DVYLQGPIWAK (SEQ ID NO: 10) and HPPPQILIK (SEQ ID NO: 11). The two peptides were clearly detected and sequenced in both modified and unmodified samples without any modifications, underlying the specificity of the chemical reaction on the cysteine residues.

In brief, the Inventors showed that the benzoylacrylamide ligand of the invention enabling a specific and effective coupling on cysteine residues present in the capsid of AAV2 and AAV9.

### Example 3: Infectivity Assay Concerning Ligand (L)

### Protocol

The infectivity of AAV2-GalNAc-acrylamide prepared in Example 2 (AAV2 chemically modified by ligand L) and that of the starting AAV2 was assessed as follows:

HeLa cells were seeded in 2 mL DMEM growth medium in 6-well culture plates at a density of 10⁶ cells/well. Cells were then incubated overnight at 37° C. to reach 50% confluence. The viral stock was then diluted 10-fold by serial dilution. Next, 2 μL of each dilution was added to separate wells in the 6-well plates. Plates were then incubated at 37° C. for 24 h. The infectivity of the AAV2-GFP was measured immediately upon thawing of the sample. The same procedure was used for AAV2-GalNAc-Acrylamide particles. AAV2-GFP-infected cells were detected by fluorescence microscopy.

The transducing unit (TU) titer was calculated using the following formula:

$$T U / mL = ( 40 \; 40 \times NGFP \times dilutions \times 1000 ) / V$$

where NGFP is the mean number of GFP-positive cells per well and V is the volume (in μL) of vector used to infect cells.

### Results:

Infectivity of AAV2-GalNAc-acrylamide (3E6 equivalents) was evaluated by measuring the ratio of vector genomes (vg) to GFP-forming units (vg/GFU) in HeLa cells. This ratio is classically used as a quality control measure to evaluate the in vitro infectivity of rAAV vectors (the higher the ratio the lower the infectivity of the vector). Non-chemically modified AAV2 was used as control.

TABLE 1
hereunder showed the results of infectivity
obtained for each type of AAV2:

| Samples | Titer (vg/mL) | GFU/mL | Vg/GFU |
|---|---|---|---|
| AAV2 | 6.6^{E}12 | 1.01^{E}8 | 6.5^{E}4 |
| AAV2-GalNAc- | 2.6^{E}12 | 1.91^{E}7 | 1.4^{E}5 |
| Acrylamide (3^{E}6) | | | |

The Vg/GFU ratio for the AAV2-GalNAc-acrylamide vector was in the same order of magnitude as that of non-chemically modified AAV2.

Thus, the results showed that AAV2-GalNAc-acrylamide vector efficiently transduced HeLa cells and that this vector remains infectious after the bio-conjugaison process.

### Example 4: Synthesis of Other Ligands According to the Invention

### Synthesis of GalNAc-Sulfonamide (L1)

### Preparation of Intermediate Compound 9

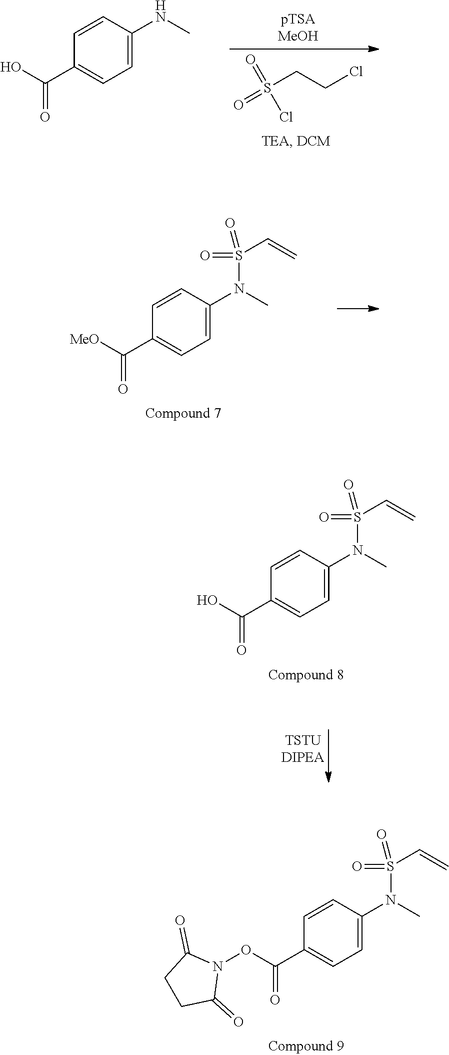 [SMILES: CNc1ccc(C(=O)O)cc1 ; O=S(=O)(Cl)CCCl ; C=CS(=O)(=O)N(C)c1ccc(C(=O)OC)cc1 ; C=CS(=O)(=O)N(C)c1ccc(C(=O)O)cc1 ; C=CS(=O)(=O)N(C)c1ccc(C(=O)ON2C(=O)CCC2=O)cc1]

### Compound 7

To a solution of the 4-(methylamino)benzoic acid (1.00 g, 6.616 mmol) in MEH (20 mL) was added pTSA (1.51 g, 7.939 mmol). The resulting mixture was stirred under reflux for 24 h. The mixture was evaporated under reduced pressure. The residue was diluted with AcOEt and washed with a saturated solution of NaHCO₃. The crude was used in the next step without further purification.

To a solution of the crude product in dry DCM (60 mL) containing TEA (4.51 mL, 33.080 mmol), was added, dropwise, and at 0° C., 2-chloroethanesulfonyl chloride (1.04 mL, 9.924 mmol). After 1 h of stirring, water was added and the compound extracted. The crude of the reaction was used in the next step without further purification (1.27 g, 4.962 mmol, 75% for two steps).

¹H NMR (300.13 MHz, CDCl3): δ_{H} 8.02 (d, 2H, J=8.8 Hz, Ar), 7.39 (d, 2H, J=8.8 Hz, Ar), 6.42 (dd, 1H, J=16.5 Hz, J=9.8 Hz, vinyl), 6.21 (d, 1H, J=16.5 Hz, vinyl), 6.03 (d, 1H, J=9.8 Hz, vinyl), 3.91 (s, 3H, COOMe), 3.27 (s, 3H, NHMe).

13C NMR (75.48 MHz, CDCl3): δ_{C} 166.2 (C, COOMe), 142.2 (C, Ar), 132.0 (CH, vinyl), 130.5 (2×CH, Ar), 129.0 (CH₂, vinyl), 128.4 (C, Ar), 125.2 (2×CH, Ar), 52.2 (CH₃), 37.5 (CH₃).

HRMS (ESI⁺): m/z calculated for C₁₁H₁₃NO₄SNa [M+Na]⁺278.0463 found 278.0458.

### Compound 9

To a solution of the crude (500 mg, 1.958 mmol) in 1,4-dioxane/H₂O, 1:1 (20 mL) was added LiOH (56 mg, 2.350 mmol). The resulting mixture was stirred at RT for 3 h. The mixture was neutralized by adding acid resin. After evaporation, the carboxylic acid 8 was obtained (HRMS-ESI-: m/z calculated for C₁₀H₁₀N₂O₄S [M−H]— 240.0331 found 240.0330).

To a solution of the carboxylic acid 8 in dry DMF (20 mL) containing DIPEA (1.71 mL, 9.79 mmol) was added, dropwise and at 0° C., TSTU (884 mg, 2.937 mmol). After 1 h of stirring at RT, the mixture was washed with a saturated solution of NaHCO₃. The residue was purified by flash chromatography (SiO2, DCM/AcOEt: 50/50 to 0/100) to yield the vinylsulfonamide derivative 9 (325 mg, 0.959 mmol, 49%) as a white solid.

¹H NMR (300.13 MHz, CDCl3): δ_{H} 8.12 (d, 2H, J=8.8 Hz, Ar), 7.47 (d, 2H, J=8.8 Hz, Ar), 6.41 (dd, 1H, J=16.5 Hz, J=9.8 Hz, vinyl), 6.22 (d, 1H, J=16.5 Hz, vinyl), 6.05 (d, 1H, J=9.8 Hz, vinyl), 3.30 (s, 3H, NHMe), 2.90 (s, 4H, NHS).

13C NMR (75.48 MHz, CDCl3): δ_{C} 169.15 (2×C, NHS), 161.0 (C, COOH), 147.0 (C, Ar), 131.9 (CH, vinyl), 131.5 (2×CH, Ar), 129.5 (CH₂, vinyl), 124.8 (2×CH, Ar), 122.8 (C, Ar), 37.2 (CH₃), 25.6 (CH₂, NHS).

HRMS (ESI⁺): m/z calculated for C₁₄H₁₄N₂O₆SNa [M+Na]⁺361.0470 found 361.0460.
Preparation of Compound GalNAc-Sulfonamide (L1) from Compounds 6 and 9

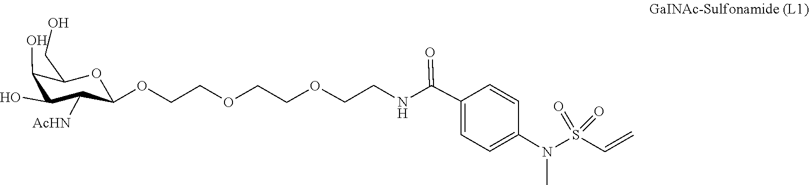

To a solution of compound 6 (see Example 1-20 mg, 0.0567 mmol) containing DIPEA (30 μL, 0.1700 mmol) in DMF (1 mL) was added a solution of compound 9 (23 mg, 0.0681 mmol) in DMF (0.5 mL). The resulting mixture was stirred at rt for 30 min. The mixture was evaporated under reduced pressure. The residue was purified by flash chromatography (SiO2, DCM/MeOH: 90/10 to 80/20) to yield the GalNAc-Sulfonamide (L1) (24 mg) as a white solid. A second purification in RP chromatography (C18 cartridge 15 μm, H2O 0.1% TFA/ACN: 95/5→90/10→80/20 as gradient eluents) yielded the pure compound GalNAc-Sulfonamide (L1) (24 mg, 0.0417 mmol, 74%).

1H NMR (300.13 MHz, MeOD, 298.15 K): δ_{H} 7.85 (m, 2H, H₁₇), 7.47 (m, 2H, H18), 6.67 (m, 1H, H21), 6.11 (m, 1H, H22), 4.41 (d, J1,2=8.3 Hz, 1H, H1), 3.99-3.45 (m, 18H), 3.27 (s, 3H, H20), 1.97 (s, 3H, NHAc).

13C NMR (75.48 MHz, MeOD, 298.15 K): δ_{C} 174.2 (C, CO, NHAc), 169.4 (C, CO), 145.8 (C, Ar), 134 (C, Ar), 133.8 (CH), 129.8 (CH2), 129.2 (CH, Ar), 127.1 (CH, Ar), 103.8 (CH, C1), 76.8 (CH, C5), 73.5 (CH, C3), 71.6 (CH2), 71.5 (CH2), 71.4 (CH2), 70.6 (CH2), 69.7 (CH, C4), 62.6 (CH2, C6), 54.3 (CH, C2), 37.7 (CH2), 38.2 (CH3) 23.1 (CH3).

HRMS (ESI⁺): m/z calculated for C₂₄H₃₇N₃O₁₁SNa [M+Na]⁺598.2046 found 598.2056.

### Synthesis of compound GalNAc-Maleimide (L2) from compound 6

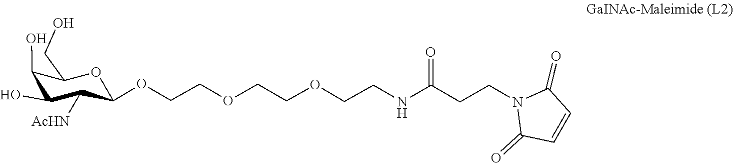

To a solution of compound 6 (see Example 1-38 mg, 0.106 mmol) containing DIPEA (55 μL, 0.318 mmol) in DMF (2 mL) was added 3-(Maleimido)propionic acid N-hydroxysuccinimide ester (34 mg, 0.127 mmol). The resulting mixture was stirred at rt for 1 h. The mixture was evaporated under reduced pressure. The residue was purified by flash chromatography (SiO2, DCM/MeOH: 90/10 to 70/30) to yield the GalNAc-Maleimide (L2) (43 mg) as a white solid. A second purification in RP chromatography (C18 cartridge 15 μm, H2O 0.1% TFA/ACN: 95/5→90/10→80/20 as gradient eluents) yielded the pure compound GalNAc-Maleimide (L2) (34 mg, 0.0675 mmol, 64%).

1H NMR (300.13 MHz, D2O, 298.15 K): δ_{H} 6.84 (s, 2H, maleimide), 4.47 (d, J1,2=8.4 Hz, 1H, H1), 3.99 (ddd, 1H, J=11.4 Hz, J=5.5 Hz, J=3.0 Hz, H5), 3.91-3.58 (m, 17H), 3.53 (t, 2H, J=5.3 Hz), 3.29 (t, 2H, J=5.3 Hz), 4.49 (t, 2H, J=6.5 Hz), 2.00 (s, 3H, NHAc).

13C NMR (75.48 MHz, D2O, 298.15 K): δ_{C} 174.6 (C, CO), 173.6 (C, CO), 172.5 (C, CO), 134.4 (CH, maleimide), 101.5 (CH, C1), 75.1 (CH, C5), 71.0 (CH), 69.7 (2×CH2), 69.4 (CH2), 68.8 (CH2), 68.6 (CH2), 67.8 (CH), 60.9 (CH), 52.3 (CH2), 38.9 (CH2), 34.6 (CH2), 34.4 (CH2), 22.2 (CH3, NHAc).

HRMS (ESI⁺): m/z calculated for C₂₁H₃₃N₃O₁₁Na [M+Na]+526.2013 found.

### Example 5: Coupling Experiments with Ligand L2 (Maleimide Ligand), Comparison with Ligand L (Benzoyl Acrylamide), and Impact of the pH on the Coupling Yield

The same coupling protocol as that shown in Example 2 was used. Two pH were used namely pH=7.3 (as used in Example 1) and pH=9.1. Briefly: AAV2-CAG-GFP (10E12 vg, 2.49 nmol, 100 μL) were added to a solution of dPBS buffer (100 μL or 900 μL) or in basic buffer 50/50 dPBS/PBS (pH=9.1) (200 μL) containing the GalNAc-Acrylamide Ligand (L) (3E6 equivalents), or the GalNAc-Maleimide Ligand (L2) (3E6 equivalents) and incubated during 4 h at RT. The solutions containing the vectors were then dialyzed against dPBS+0.001% Pluronic to remove free molecules that were not bond to the AAV2 capsid. The resulting AAV2 were characterized by dot blot analysis using immunostaining with A20 antibody in order to assess the capsid integrity and staining with soybean lectin to selectively detect GalNAC and thus the coupling efficiency for each tested ligand.

Results:

The results of dot blot analysis are shown in FIGS. 5A and 5B. The integrity of the capsids after coupling reaction with the ligand L (benzoyl acrylamide ligand) or the ligand L2 (maleimide ligand) was assessed by dot blot analysis using immunostaining with A20 antibody. A20 antibody recognizes the assembled AAV2 capsid. The positive dots with A20 antibody indicate that AAV2 remained intact after the coupling procedure with Ligand L or Ligand L2 regardless the coupling pH.

Dot blot analysis with soybean lectin, which selectively binds to GalNA,c showed clear positive dots for ligand L, confirming the covalent coupling (as also evidenced by HPLC/MC analysis in Example 3). Surprisingly, the intensity of dots with soybean lectin staining was more intense for AAV2 coupled with ligand L at pH 9.1 than for AAV2 coupled with L at pH 7.4. Such a result supports that basic pH increases the efficacy of coupling, namely the number of cysteine residues in the capsid chemically modified with L. In other words, by modulating the pH of coupling, it is possible to tune the proportion of cysteine residues chemically modified with acrylamide ligands in the AAV capsid.

The intensity of dots with soybean lectin staining obtained with ligand L2 (maleimide) is lower than that observed for AAV2 chemically coupled with L. At pH 7.3, the intensity of the dot for L2 is weak and even similar to the negative control (AAV2) suggesting, at best, a very low proportion of chemically modified cysteine residues present in the AAV2 capsid. For the coupling performed with L2 at pH 9.1, the dot for L2 is more visible and more intense than at pH 7.3 suggesting a better chemical coupling. However, the dot for L2 at pH 9.1 is much less intense than that observed with L.

In other words, L2 ligand (maleimide) appears to be significantly less effective than acrylamide ligands to chemically modify cysteine residues in the capsid of AAV, regardless the pH used for the coupling. The low coupling efficiency of the maleimide ligand L can be explained, at least in part, by the instability of the coupling function in aqueous buffer due to retro-Michael reaction.

1-24. (canceled)

25. An adeno-associated Virus (AAV) having at least one chemically-modified cysteine residue in its capsid, wherein said chemically-modified cysteine residue is of formula (I): 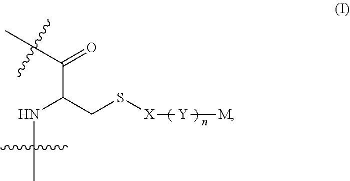
wherein:
X is selected from the group consisting of: 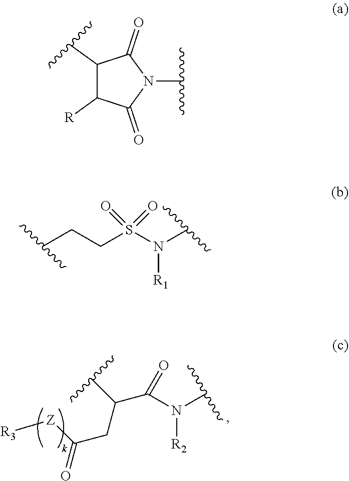
wherein
Z is —O—, —S—, or —N(R₄)—,
R₁, R₂, R₃ and R₄ are each independently selected from a hydrogen atom, an alkyl group, an aryl group, a heteroaryl, said group being optionally substituted,
k is 0 or 1,
R is a hydrogen, a halogen, an alkyl group, an aryl group, a heteroaryl group, an alkoxy group, said group being optionally substituted,
Y is a spacer,
n is 0 or 1, and
M is a functional moiety.

26. The AAV of claim 25, wherein X is of formula (b) or formula (c).

27. The AAV of claim 25, wherein the chemically-modified cysteine residue is of formula (I):

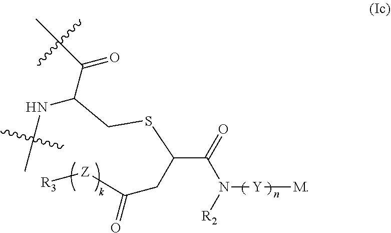

28. The AAV of claim 27, wherein:
R₂ is hydrogen atom, a C1-C6 alkyl group, an aryl group comprising from 6 to 14 ring atoms, or a heteroaryl group comprising from 5 to 14 ring atoms, and/or
R₃ is selected from the group consisting of an aryl group comprising from 6 to 14 ring atoms and heteroaryl group comprising from 5 to 14 ring atoms, said aryl or heteroaryl group being optionally substituted by 1 to 3 substituents selected from halogens, —OH, NH₂, NO₂, C1-C3 alkyl, C1-C3 alkoxy, C1-C3 hydroxyalkyl, and C1-C3 haloalkyl, and/or
k is 0.

29. The AAV of claim 27, wherein:
R₂ is hydrogen atom or a C1-C6 alkyl group,
R₃ is an unsubstituted phenyl or a phenyl substituted by 1 to 3 substituents selected from halogens, —OH, NH₂, NO₂, C1-C3 alkyl, C1-C3 alkoxy, C1-C3 hydroxyalkyl, and C1-C3 haloalkyl, and
k is 0.

30. The AAV of claim 25,
wherein Y is a spacer of formula (II): 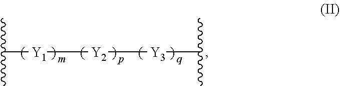
wherein:
m, p and q are each independently 0 or 1,
Y₁ is selected from the group consisting of an alkylene group, an arylene group, a heteroarylene group, said group being optionally substituted,
Y₂ is —C(═O)—NH, —C(═O)—O, —C(═O)—O—C(═O)—, O—(C═O)—, NH—C(═O)—, NH—C(═O)—NH, —O—C═O—O—, O, NH, —NH(C═S)—, or —(C═S)—NH—,
Y₃ is selected from the group consisting of polymers, homopolymers, copolymers and block polymers, peptides, oligosaccharides, saturated or unsaturated, branched or linear hydrocarbon chains, optionally interrupted by one or several heteroatoms and/or by a group selected from —C(═O)—NH, —C(═O)—O, —C(═O)—O—C(═O)—, O—(C═O)—, NH—C(═O)—, NH—C(═O)—NH, —O—C(═O)—O—, —NH(C═S)—, and —(C═S)—NH-, and/or by one or more C3-C6 hydrocarbon cycle or C2-C6 heterocycle.

31. The AAV of claim 25, wherein M is a functional moiety comprising a group selected from a click-chemistry group, a steric shielding agent, a labelling agent, a targeting agent a drug moiety, an oligonucleotide and combinations thereof.

32. The AAV of claim 26, wherein said chemically-modified cysteine residue is of formula (I) wherein:
X is of formula (c),
n is 1,
M is a functional moiety, and
Y is a spacer of formula (II) wherein m is 0, p is 0, q is 1 and Y₃ is selected from the group consisting of saturated or unsaturated, linear or branched C2-C40 hydrocarbon chains, optionally substituted, polyethylene glycol, polypropylene glycol, pHPMA, PLGA, polymers of alkyl diamines and combinations thereof.

33. The AAV of claim 26, wherein:
Y is a spacer of formula (II) wherein, q is 1 and Y₃ is selected from the group consisting of saturated or unsaturated, linear or branched C2-C40 hydrocarbon chains, optionally substituted, polyethylene glycol, polypropylene glycol, pHPMA, PLGA, polymers of alkyl diamines and combinations thereof, and/or
M comprises a click-chemistry group, an oligonucleotide, a vitamin, a drug, a targeting agent selected from a mono-saccharide, a polysaccharide, a hormone, a peptide, a membrane receptor or a fragment thereof, an aptamer, an antibody and fragments thereof, a ScFv, a spiegelmer, and a peptide aptamer.

34. The AAV of claim 26, wherein:
Y is a spacer of formula (II) wherein, q is 1, and Y₃ is selected from the group consisting of linear or branched C2-C20 alkyl chains, polyethylene glycol, polypropylene glycol, pHPMA, PLGA, polymer of alkyl diamine and combinations thereof, said polymers having from 2 to 20 monomers and/or
“M” comprises a vitamin or a targeting agent selected from a ligand derived from a protein selected from transferrin, Epidermal Growth Factor (EGF), and basic Fibroblast Growth Factor BFGF, a mono- or a polysaccharide comprising one or several galactose, mannose, N-acetylgalactosamine residues, bridge GalNac, or mannose-6-phosphate, sialic acid and derivatives thereof, and MTP selected from SEQ ID NO:1 to SEQ ID NO:7.

35. The AAV of claim 25, wherein:
M is a cell-type specific ligand for specifically targeting hepatocytes and comprises at least one moiety of formula (III): 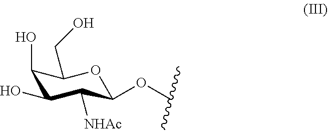
Y is a polyethylene glycol chain comprising from 2 to 10 monomers.

36. The AAV of claim 35, wherein the at least one chemically-modified cysteine in the capsid, is of formula (Ic-1):

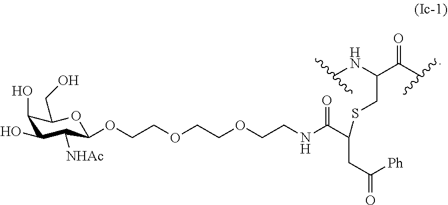

37. The AAV of claim 25, which further has at least one additional chemically modified amino acid residue in the capsid, which is different from a cysteine residue, said amino acid residue bearing:
a modified amino group of formula (V): 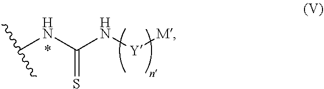
wherein:
N* being the nitrogen of the amino group of an amino acid residue, e.g. of a lysine residue or arginine residue,
Y′ is a spacer,
n′ is 0 or 1, and
M′ is a functional moiety; or
a modified tyrosyl residue of formula (VI): 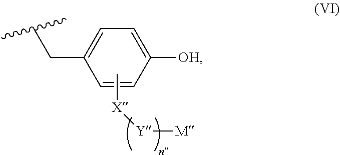
wherein:
X″ is —N═N— or 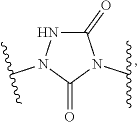 [SMILES: *n1[nH]c(=O)n(*)c1=O]
Y″ is a spacer,
n″ is 0 or 1, and
M″ is a functional moiety.

38. The AAV of claim 25, wherein the AAV is a recombinant AAV having a wildtype capsid, naturally-occurring serotype AAV, variant AAV, pseudotype AAV, AAV with hybrid, or a self-complementary AAV.

39. A method for chemically-modifying the capsid of an AAV which comprises incubating said AAV with a chemical reagent bearing a reactive group selected from a maleimide, a vinyl sulfonamide and a 3-(carboxy derivative)acrylamide in conditions conducive for reacting said reactive group with a cysteine residue present in the capsid of the AAV so as to form a covalent bound.

40. The method of claim 39, which comprises incubating the AAV with a chemical reagent of formula (VIIc): 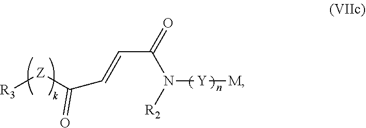
so as to obtain at least one chemically-modified cysteine residue in the capsid of formula (Ic): 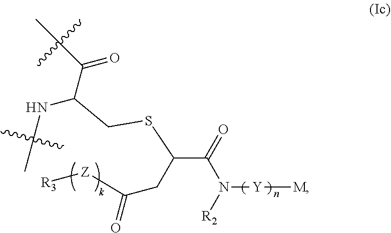
wherein:
Y is a spacer, n is 0 or 1, M is a functional moiety,
Z is —O—, —S—, or —N(R₄)—,
k is 0 or 1, and
R₂, R₃ and R₄ are each independently selected from a hydrogen atom, an alkyl group, an aryl group, and a heteroaryl group, said group being optionally substituted.

41. The method of claim 40, wherein
Y is a spacer of formula (II): 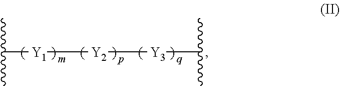
in which m is 0, p is 0, q is 1 and Y₃ is selected from the group consisting of saturated or unsaturated, linear or branched C2-C40 hydrocarbon chains, optionally substituted, polyethylene glycol, polypropylene glycol, pHPMA, PLGA, polymers of alkyl diamines and combinations thereof, and
M is a click-chemistry group, a steric shielding agent, a labelling agent, a targeting agent or a drug moiety.

42. The method of claim 39, wherein the incubation step is performed at a pH from 5.0 to 11.

43. An AAV obtainable by the method of claim 39.

44. A pharmaceutical composition comprising an AAV as defined in claim 25 and at least one pharmaceutically acceptable excipient.

45. A gene vector comprising an AAV of claim 25, said AAV further comprising a transgene sequence in its viral genome.

46. A method of gene therapy comprising the administration of the gene vector of claim 45 to a cell, said transgene encoding a therapeutic protein.

47. A method of diagnosis, said method comprising administering an AAV of claim 25, said AAV comprising an imaging agent, to a subject and visualizing the imaging agent.
